(19) United States
(12) Patent Application Publication
(10) Pub. No.: US 2013/0115596 A1
(43) Pub. Date: May 9, 2013

(54) DNA POLYMORPHISMS AS MOLECULAR MARKERS IN CATTLE

(71) Applicants: Stephen Moore, Edmonton (CA); Graham Plastow, Edmonton (CA); Luiz Heraldo Arouche Camara-Lopes, Sao Paulo (BR); Flavio Canellas Canavez, Sao Paulo (BR); Paulo Sérgio Lopes Oliveria, Sao Paulo (BR); Katia Ramos Moreira Leite, Sao Paulo (BR)

(72) Inventors: Stephen Moore, Edmonton (CA); Graham Plastow, Edmonton (CA); Luiz Heraldo Arouche Camara-Lopes, Sao Paulo (BR); Flavio Canellas Canavez, Sao Paulo (BR); Paulo Sérgio Lopes Oliveria, Sao Paulo (BR); Katia Ramos Moreira Leite, Sao Paulo (BR)

(73) Assignees: GENOA BIOTECNOLOGIA S.A., Sao Paulo (BR); THE GOVERNORS OF THE UNIVERSITY OF ALBERTA, Edmonton, Alberta (CA)

(21) Appl. No.: 13/637,301

(22) PCT Filed: Mar. 25, 2011
(86) PCT No.: PCT/CA2011/000306

§ 371 Date: Dec. 15, 2012

Related U.S. Application Data
(60) Provisional application No. 61/317,665, filed on Mar. 25, 2010.

(51) Int. Cl. C12Q 1/68

(52) U.S. Cl. CPC C12Q 1/6876
USPC 435/6.11

(57) ABSTRACT

A method of predicting the phenotype of cattle through the analysis of one or more single nucleotide polymorphisms (SNPs) is described. More particularly, a method for predicting cattle temperament and behavior through the analysis of one or more single nucleotide polymorphisms (SNPs) mapped at specific regions of the bovine genome is described.

### PRIORITY

This application claims priority of U.S. Provisional Patent Application No. 61/317,665 filed Mar. 25, 2010 and hereby incorporates the same provisional application by reference herein in its entirety.

### FIELD

The technical field is a method of predicting the phenotype of cattle through the analysis of one or more single nucleotide polymorphisms (SNPs). More particularly, the technical field is a method for predicting cattle temperament and behavior through the analysis of one or more SNPs mapped at specific regions of the bovine genome is described.

### BACKGROUND

In animals such as cattle, intolerance to stress may have detrimental effects on the health of the animal. For example, high stress may cause weight loss and affect cell functioning. These effects may have detrimental effects on meat production, flavor and tenderness. Accordingly, researchers have attempted to determine the genetic loci which are associated with stress levels in these animals.

One approach that may be used to identify these loci is quantitative trait analysis. Quantitative trait loci (QTLs) are stretches of DNA that are closely linked to the genes that underlie the trait in question. Factors such as genetic heterogeneity, epistasis, low penetrance, variable expressiveness, and pleiotropy may contribute to the polygenic or quantitative inheritance of a phenotypic trait. Therefore, unlike monogenic traits, polygenic traits do not follow patterns of Mendelian inheritance. This hinders the identification of the genes and the alleles responsible for the variations observed among individuals of one species. Association studies based on more robust genomic methodologies are accelerating the analyses of QTLs, thereby abbreviating the time required to identify QTLs related to a characteristic of interest.

The parametric approach in association studies requires the identification of risk markers a priori, in the case of single nucleotide DNA polymorphisms (SNPs). Genomic characterization projects have generated enormous amount of data which are available in public databanks. The genomic sequences may be compared and reorganized by bioinformatics analyses. For example, a bank of bovine QTLs have been made available by Polineni et al. (BMC Bioinformatics. 2006 Jun. 5; 7:283.).

In general, due to the high cost associated with determining QTLs, public databases are limited to information arising from the genomic characterization of a few individuals. The application of new experimental platforms to the data already available in these public databases may allow for the simultaneous evaluation of dozens of thousands of SNPs at reduced costs. Computational techniques and statistics may allow for the analyses of genotypical data, and for a correlation to be made between the genotypes and the phenotypes of interest.

Accordingly, there is a need to identify new computational techniques for the identification of genetic loci involved in traits such as tolerance to stress.

There is also the need to identify loci that have potential roles in the determination of traits such as temperament, tolerance to stress and behaviour in animals such as cattle.

### SUMMARY

Methods for predicting animal behaviour through the use of SNPs are described. More particularly, methods of correlating a particular phenotypic trait with a SNPs in cattle are described. The methods can be used, for example, to predict whether the phenotypic trait is present in a particular animal or group of animals.

Broadly stated, a method of predicting the phenotype of an animal is provided, the method comprising: selecting a phenotypic trait in a population of animals; determining single nucleotide polymorphisms in the genotype of the population of animals, correlating the single nucleotide polymorphisms with the phenotypic trait, and predicting the phenotype of the animal based on the results of the correlation.

Broadly stated, a method of predicting the tolerance of a cow to stress is provided, the method comprising: determining cortisol levels in a population of cattle; determining single nucleotide polymorphisms in the cattle genome; correlating the single nucleotide polymorphisms with the cortisol levels in the cattle; and predicting the cortisol level in a cow based on the results of the correlation.

Broadly stated, a method for predicting a phenotypic trait in a cow is provided, the method comprising: determining the nucleotide present at a locus selected from the group consisting of ARS-BFGL-NGS-102860 mapped at position 36,875,752 (Btau4.0) of bovine chromosome 16 (BTA16); ARS-BFGL-NGS-119018 mapped at position 104,533,532 (Btau4.0) of bovine chromosome 11 (BTA11), ARS-BFGL-NGS-20850 at position 7,928,145 (Btau4.0) of bovine chromosome 14 (BTA14), ARS-BFGL-NGS-100843 mapped at position 45,768,092 (Btau4.0) of bovine chromosome 16 (BTA16), ARS-BFGL-NGS-97162 mapped at position 51,027,089 (Btau4.0) of bovine chromosome 16 (BTA16), Hapmap42294-BTA-69421 at position 7,311,099 (Btau4.0) of bovine chromosome 3 (BTA3), ARS-BFGL-BAC-2384 at position 31,838,306 (Btau4.0) of bovine chromosome 19 (BTA19), BTB-01553536 at position 103,411,819 (Btau4.0) of bovine chromosome 7 (BTA7) Hapmap53129-rs29022984 at position 97,865,487 (Btau4.0) of bovine chromosome 11 (BTA11); ARS-BFGL-NGS-68110 mapped at position 106,356,144 (Btau4.0) of bovine chromosome 11 (BTA11); Hapmap49592-BTA-38891 at position 36,808,659 (Btau4.0) of bovine chromosome 16 (BTA16); ARS-BFGL-NGS-30157 mapped at position 108,365,498 (Btau4.0) of bovine chromosome 11 (BTA11); Hapmap30097-BTC-007678 mapped at position 7,969,430 (Btau4.0) of bovine chromosome 14 (BTA14); ARS-BFGL-NGS-82206 mapped at position 130,073,477 (Btau4.0) of bovine chromosome 1, ARS-BFGL-NGS-114897 mapped at position 69,718,192 (Btau4.0) of bovine chromosome 11 (BTA11), ARS-BFGL-NGS-32646 mapped at position 103,515,296 (Btau4.0) of bovine chromosome 11 (BTA11); ARS-BFGL-NGS-12135 mapped at position 106,208,942 (Btau4.0) of bovine chromosome 11 (BTA11), BTA-98582-no-rs mapped at position 72,891,230 (Btau4.0) of bovine chromosome 15 (BTA15), Hapmap50501-BTA-91866 mapped at position 16,697,957 (Btau4.0) of bovine chromosome 16 (BTA16), ARS-BFGL-NGS-55834 mapped at position 18,500,742 (Btau4.0) of bovine chromosome 16 (BTA16), ARS-BFGL-NGS-43639 at position 45,798,238 (Btau4.0) of bovine chromosome 16 (BTA16), ARS-BFGL-NGS-114602 mapped at position 2,011,968 (Btau4.0) of bovine chromosome 20 (BTA20), ARS-BFGL-NGS-10830 mapped at position 14,303,665 (Btau4.0) of bovine chromosome 21 (BTA21), ARS-BFGL-BAC-35732 mapped at position 37,243,031 (Btau4.0) of bovine chromosome 22 (BTA22), BTB-00000725 mapped at position 19,405,585 (Btau4.0) of bovine chromosome 27 (BTA27), Hapmap32414-BTA-65998 mapped at position 38,481,013 (Btau4.0) of bovine chromosome 28 (BTA28), Hapmap26724-BTA-152272 mapped at position 126,295,740 (Btau4.0) of bovine chromosome 1 (BTA1), ARS-BFGL-NGS-27655 mapped at position 3,683,167 (Btau4.0) of bovine chromosome 3 (BTA3), ARS-BFGL-NGS-112731 mapped at position 4,206,765 (Btau4.0) of bovine chromosome 2 (BTA2), Hapmap42580-BTA-54259 mapped at position 38555445 (Btau4.0) of bovine chromosome 22 (BTA22), BTB-01548453 mapped at position 103,511,536 (Btau4.0) of bovine chromosome 7 (BTA7), INRA-453 mapped at position 20,719,615 (Btau4.0) of bovine chromosome 3 (BTA3), BTB-00186413 mapped at position 58,422,144 (Btau4.0) of bovine chromosome 4 (BTA4)(G), UA-IFASA-7842 at position 7,857,978 (Btau4.0) of bovine chromosome 14 (BTA14), BTB-01944037 at position 112,370,482 (Btau4.0) of bovine chromosome 8 (BTA8), BTB-00086583 at position 26,641,920 (Btau4.0) of bovine chromosome 2 (BTA2), ARS-BFGL-NGS-111311 at position 51,300,416 (Btau4.0) of bovine chromosome 23 (BTA23), BTB-01570493 at position 25,395,611 (Btau4.0) of bovine chromosome 8 (BTA8), ARS-BFGL-NGS-104914 at position 125,588,038 (Btau4.0) of bovine chromosome 5 (BTA5). BTA-114011-no-rs at position 125,911,737 (Btau4.0) of bovine chromosome 1 (BTA1), ARS-BFGL-NGS-23375 at position 40,238,627 (Btau4.0) of bovine chromosome 24 (BTA24), ARS-BFGL-NGS-78666 at position 136,573,912 (Btau4.0) of bovine chromosome 1 (BTA1), BTB-01087838 at position 89,620,818 (Btau4.0) of bovine chromosome 10 (BTA10), Hapmap31564-BTC-007633 at position 7,998,737 (Btau4.0) of bovine chromosome 14 (BTA14), Hapmap50402-BTA-58146 at position 42,593,193 (Btau4.0) of bovine chromosome 24 (BTA24), and ARS-BFGL-BAC-46971 at position 35,184,932 (Btau4.0) of bovine chromosome 25 (BTA25), either alone or in combination with other loci, and predicting the phenotypic trait in the cow comprising based on the nucleotide present at the locus.

### BRIEF DESCRIPTION OF THE DRAWINGS

FIG. 1 is a graph showing the distribution of cortisol levels in a sample of 1,189 cows from Farm Jacarezinho;

FIG. 2 is a graph showing the distribution of flight speed in a sample of 1,189 cows kept at Farm Jacarezinho;

FIG. 3 is a graph showing the relationship between cortisol levels and flight speed in cows at Farm Jacarezinho;

FIG. 4 is a graph showing the distribution of SNPs in chromosomes 11 (left) and 16 (right), and the level of association using log-additive genetic model;

FIG. 5 is a Q-Q plot showing deviations from normality for cortisol levels measured in 1,799 Nelore sires from different regions of Brazil; and

FIG. 6 is a density plot of cortisol levels in cattle sampled in various farms in Brazil, and a descriptive statistics per farm.

### DETAILED DESCRIPTION

Methods of determining a feature of animal behaviour is described. In particular, the method relates to the determination of particular phenotypes in cattle using SNPs. Once SNPs related to particular traits are identified, the presence or absence of a particular SNP may be used as a marker as to whether a particular cow or cows will possess the phenotypic trait in question.

According to some embodiments of the method, a phenotypic trait of interest can be identified in a population of animals, such as cattle. The trait can show a normal distribution in the population of animals, although this is not necessary. A normal distribution can identify traits not selected by breeders. The trait can be multigenic (e.g. the expression of the trait can be determined by multiple genes). For example, cortisol levels can be determined. The genotypes of the animals expressing the trait can then be determined with a view to identifying SNPs present in the genome. Once the genotypes are determined, the presence of a SNP can be correlated with the presence of the trait. For example, a general linear model can be used to correlate the presence of a given SNP with a trait. Based on the results of association analysis, potential loci involved in the phenotypic expression of the trait can be identified. These loci can be subjected to genomic characterization to determine nucleotide variations. The allele frequency present at a given loci can be determined. The presence or absence of an allele in a particular animal can then be used as a predictor of the animal's behaviour.

According to an embodiment of the method, there is provided a method of predicting the phenotype of an animal. The method can comprise selecting a phenotypic trait in a population of animals; determining the single nucleotide polymorphisms in the genotype of the population of animals, correlating the single nucleotide polymorphisms with the phenotypic trait, and predicting the phenotype of the animal based on the results of the correlation.

According to another embodiment of the method, there is provided a method of predicting the tolerance of a cow to stress. The method can comprise determining, for example, the cortisol levels in a population of cattle, determining the single nucleotide polymorphisms in the cattle genome, correlating the single nucleotide polymorphisms with the cortisol levels in the cattle, and predicting the cortisol level in a cow based on the results of the correlation. As would be apparent to one skilled in the art, it is contemplated that other markers of stress can also be examined.

According to another aspect of the method, there is provided a method as described herein, wherein the step of determining the nucleotide present in each allele of that individual in the selected locations can be performed by genomic DNA sequencing of that region. Such sequencing can be done in a manner that would be known to one skilled in the art.

According to another aspect of the method, there is provided a method as defined herein, wherein the step of determining the nucleotide present in each allele of the animal at the selected location can be accomplished by (a) amplifying a region of genomic DNA that includes the given position to generate an amplicon, and (b) treating the amplicon with a restriction enzyme enzyme in its corresponding buffer to determine the identity of the nucleotides present in the selected location. Such amplification and restriction analysis can be done in a manner that would be known to one skilled in the art.

According to another aspect of the method, there is provided a method as defined herein, wherein the step of determining the nucleotide present in the allele of the animal at the selected location can be accomplished by (a) amplifying a region of genomic DNA that includes the given position to generate an amplicon, and (b) hybridization of the amplified probes specific to the selected location, where hybridization determines the identity of the nucleotides present. Such amplification and hybridization can be done in a manner that would be known to one skilled in the art.

As would be apparent to one skilled in the art, the term ‘hybridization’ as used herein can include probe hybridization of a DNA fragment that can recognize an allele present in a specific genomic region. A DNA probe can be used in, but not limited to, experiments such as microarray DNA, southern blotting, real time PCR, among others. Hybridization conditions can vary between each methodology as would be apparent to one skilled in the art.

According to one aspect of the method, there is provided a method for predicting cattle animal behavior by determining the nucleotide present at locus ARS-BFGL-NGS-102860 alone or combine with any other cattle loci. This locus can be mapped at position 36,875,752 (Btau4.0) of bovine chromosome 16 (BTA16) where either Cytosine (C) or Thymine (T) can be found.

According to another aspect of the method, there is provided a method for predicting cattle animal behavior by determining the nucleotide present at locus ARS-BFGL-NGS-119018 alone or combine with any other cattle loci.

This locus can be mapped at position 104,533,532 (Btau4.0) of bovine chromosome 11 (BTA11) where either Adenosine (A) or Guanidine (G) can be found.

According to another aspect of the method, there is provided a method for predicting cattle animal behavior by determining the nucleotide present at locus ARS-BFGL-NGS-20850 alone or combine with any other cattle loci. This locus can be mapped at position 7,928,145 (Btau4.0) of bovine chromosome 14 (BTA14) where either Thymine (T) or Cytosine (C) can be found.

According to another aspect of the method, there is provided a method for predicting cattle animal behavior by determining the nucleotide present at locus ARS-BFGL-NGS-100843 alone or combine with any other cattle loci. This locus can be mapped at position 45,768,092 (Btau4.0) of bovine chromosome 16 (BTA16) where either Guanidine (G) or Adenosine (A) can be found.

According to another aspect of the method, there is provided a method for predicting cattle animal behavior by determining the nucleotide present at locus ARS-BFGL-NGS-97162 alone or combine with any other cattle loci. This locus can be mapped at position 51,027,089 (Btau4.0) of bovine chromosome 16 (BTA16) where either Cytosine (C) or Thymine (T) can be found.

According to another aspect of the method, there is provided a for predicting cattle animal behavior by determining the nucleotide present at locus Hapmap42294-BTA-69421 alone or combine with any other cattle loci. This locus can be mapped at position 7,311,099 (Btau4.0) of bovine chromosome 3 (BTA3) where either Adenosine (A) or Guanidine (G) can be found.

According to another aspect of the method, there is provided a method for predicting cattle animal behavior by determining the nucleotide present at locus ARS-BFGL-BAC-2384 alone or combine with any other cattle loci. This locus can be mapped at position 31,838,306 (Btau4.0) of bovine chromosome 19 (BTA19) where either Thymine (T) or Guanidine (G) can be found.

According to another aspect of the method, there is provided a method for predicting cattle animal behavior by determining the nucleotide present at locus BTB-01553536 alone or combine with any other cattle loci. This locus can be mapped at position 103,411,819 (Btau4.0) of bovine chromosome 7 (BTA7) where either Thymine (T) or Cytosine (C) can be found.

According to another aspect of the method, there is provided a method for predicting cattle animal behavior by determining the nucleotide present at locus Hapmap53129-rs29022984 alone or combine with any other cattle loci. This locus can be mapped at position 97,865,487 (Btau4.0) of bovine chromosome 11 (BTA11) where either Adenosine (A) or Guanidine (G) can be found.

According to another aspect of the method, there is provided a method for predicting cattle animal behavior by determining the nucleotide present at locus ARS-BFGL-NGS-68110 alone or combine with any other cattle loci. This locus can be mapped at position 106,356,144 (Btau4.0) of bovine chromosome 11 (BTA11) where either Adenosine (A) or Cytosine (C) can be found.

According to another aspect of the method, there is provided a method for predicting cattle animal behavior by determining the nucleotide present at locus Hapmap49592-BTA-38891 alone or combine with any other cattle loci. This locus can be mapped at position 36,808,659 (Btau4.0) of bovine chromosome 16 (BTA16) where either Thymine (T) or Cytosine (C) can be found.

According to another aspect of the method, there is provided a method for predicting cattle animal behavior by determining the nucleotide present at locus ARS-BFGL-NGS-30157 alone or combine with any other cattle loci. This locus can be mapped at position 108,365,498 (Btau4.0) of bovine chromosome 11 (BTA11) where either Guanidine (G) or Adenosine (A) can be found.

According to another aspect of the method, there is provided a method for predicting cattle animal behavior by determining the nucleotide present at locus Hapmap30097-BTC-007678 alone or combine with any other cattle loci. This locus can be mapped at position 7,969,430 (Btau4.0) of bovine chromosome 14 (BTA14) where either Cytosine (C) or Thymine (T) can be found.

According to another aspect of the method, there is provided a method for predicting cattle animal behavior by determining the nucleotide present at locus ARS-BFGL-NGS-82206 alone or combine with any other cattle loci. This locus can be mapped at position 130,073,477 (Btau4.0) of bovine chromosome 1 (BTA1) where either Adenosine (A) or Guanidine (G) can be found.

According to another aspect of the method, there is provided a method for predicting cattle animal behavior by determining the nucleotide present at locus ARS-BFGL-NGS-114897 alone or combine with any other cattle loci. This locus can be mapped at position 69,718,192 (Btau4.0) of bovine chromosome 11 (BTA11) where either Adenosine (A) or Guanidine (G) can be found.

According to another aspect of the method, there is provided a method for predicting cattle animal behavior by determining the nucleotide present at locus ARS-BFGL-NGS-32646 alone or combine with any other cattle loci. This locus can be mapped at position 103,515,296 (Btau4.0) of bovine chromosome 11 (BTA11) where either Adenosine (A) or Guanidine (G) can be found.

According to another aspect of the method, there is provided a method for predicting cattle animal behavior by determining the nucleotide present at locus ARS-BFGL-NGS-12135 alone or combine with any other cattle loci. This locus can be mapped at position 106,208,942 (Btau4.0) of bovine chromosome 11 (BTA11) where either Cytosine (C) or Thymine (T) can be found.

According to another aspect of the method, there is provided a method for predicting cattle animal behavior by determining the nucleotide present at locus BTA-98582-no-rs alone or combine with any other cattle loci. This locus can be mapped at position 72,891,230 (Btau4.0) of bovine chromosome 15 (BTA15) where either Cytosine (C) or Thymine (T) can be found.

According to another aspect of the method, there is provided a method for predicting cattle animal behavior by determining the nucleotide present at locus Hapmap50501-BTA-91866 alone or combine with any other cattle loci. This locus can be mapped at position 16,697,957 (Btau4.0) of bovine chromosome 16 (BTA16) where either Thymine (T) or Cytosine (C) can be found.

According to another aspect of the method, there is provided a method for predicting cattle animal behavior by determining the nucleotide present at locus ARS-BFGL-NGS-55834 alone or combine with any other cattle loci. This locus can be mapped at position 18,500,742 (Btau4.0) of bovine chromosome 16 (BTA16) where either Guanidine (G) or Adenosine (A) can be found.

According to another aspect of the method, there is provided a method for predicting cattle animal behavior by determining the nucleotide present at locus ARS-BFGL-NGS-43639 alone or combine with any other cattle loci. This locus can be mapped at position 45,798,238 (Btau4.0) of bovine chromosome 16 (BTA16) where either Cytosine (C) or Thymine (T) can be found.

According to another aspect of the method, there is provided a method for predicting cattle animal behavior by determining the nucleotide present at locus ARS-BFGL-NGS-114602 alone or combine with any other cattle loci. This locus can be mapped at position 2,011,968 (Btau4.0) of bovine chromosome 20 (BTA20) where either Guanidine (G) or Adenosine (A) can be found.

According to another aspect of the method, there is provided a method for predicting cattle animal behavior by determining the nucleotide present at locus ARS-BFGL-NGS-10830 alone or combine with any other cattle loci. This locus can be mapped at position 14,303,665 (Btau4.0) of bovine chromosome 21 (BTA21) where either Cytosine (C) or Guanidine (G) can be found.

According to another aspect of the method, there is provided a method for predicting cattle animal behavior by determining the nucleotide present at locus ARS-BFGL-BAC-35732 alone or combine with any other cattle loci. This locus can be mapped at position 37,243,031 (Btau4.0) of bovine chromosome 22 (BTA22) where either Cytosine (C) or Thymine (T) can be found.

According to another aspect of the method, there is provided a method for predicting cattle animal behavior by determining the nucleotide present at locus BTB-00000725 alone or combine with any other cattle loci. This locus can be mapped at position 19,405,585 (Btau4.0) of bovine chromosome 27 (BTA27) where either Cytosine (C) or Thymine (T) can be found.

According to another aspect of the method, there is provided a method for predicting cattle animal behavior by determining the nucleotide present at locus Hapmap32414-BTA-65998 alone or combine with any other cattle loci. This locus can be mapped at position 38,481,013 (Btau4.0) of bovine chromosome 28 (BTA28) where either Cytosine (C) or Thymine (T) can be found.

According to another aspect of the method, there is provided a method for predicting cattle animal behavior by determining the nucleotide present at locus Hapmap26724-BTA-152272 alone or combine with any other cattle loci. This locus can be mapped at position 126,295,740 (Btau4.0) of bovine chromosome 1 (BTA1) where either Thymine (T) or Cytosine (C) can be found.

According to another aspect of the method, there is provided a method for predicting cattle animal behavior by determining the nucleotide present at locus ARS-BFGL-NGS-27655 alone or combine with any other cattle loci. This locus can be mapped at position 3,683,167 (Btau4.0) of bovine chromosome 3 (BTA3) where either Adenosine (A) or Guanidine (G) can be found.

According to another aspect of the method, there is provided a method for predicting cattle animal behavior by determining the nucleotide present at locus ARS-BFGL-NGS-112731 alone or combine with any other cattle loci. This locus can be mapped at position 4,206,765 (Btau4.0) of bovine chromosome 2 (BTA2) where either Guanidine (G) or Adenosine (A) can be found.

According to another aspect of the method, there is provided a method for predicting cattle animal behavior by determining the nucleotide present at locus Hapmap42580-BTA-54259 alone or combine with any other cattle loci. This locus can be mapped at position 38555445 (Btau4.0) of bovine chromosome 22 (BTA22) where either Guanidine (G) or Adenosine (A) can be found.

According to another aspect of the method, there is provided a method for predicting cattle animal behavior by determining the nucleotide present at locus BTB-01548453 alone or combine with any other cattle loci. This locus can be mapped at position 103,511,536 (Btau4.0) of bovine chromosome 7 (BTA7) where either Guanidine (G) or Thymine (T) can be found.

According to another aspect of the method, there is provided a method for predicting cattle animal behavior by determining the nucleotide present at locus INRA-453 alone or combine with any other cattle loci. This locus can be mapped at position 20,719,615 (Btau4.0) of bovine chromosome 3 (BTA3) where either Adenosine (A) or Guanidine (G) can be found.

According to another aspect of the method, there is provided a method for predicting cattle animal behavior by determining the nucleotide present at locus BTB-00186413 alone or combine with any other cattle loci. This locus can be mapped at position 58,422,144 (Btau4.0) of bovine chromosome 4 (BTA4) where either Adenosine (A) or Guanidine (G) can be found.

According to another aspect of the method, there is provided a method for predicting cattle animal behavior by determining the nucleotide present at locus UA-IFASA-7842 alone or combine with any other cattle loci. This locus can be mapped at position 7,857,978 (Btau4.0) of bovine chromosome 14 (BTA14) where either Guanidine (G) or Thymine (T) can be found.

According to another aspect of the method, there is provided a method for predicting cattle animal behavior by determining the nucleotide present at locus BTB-01944037 alone or combine with any other cattle loci. This locus can be mapped at position 112,370,482 (Btau4.0) of bovine chromosome 8 (BTA8) where either Guanidine (G) or Adenosine (A) can be found.

According to another aspect of the method, there is provided a method for predicting cattle animal behavior by determining the nucleotide present at locus BTB-00086583 alone or combine with any other cattle loci. This locus can be mapped at position 26,641,920 (Btau4.0) of bovine chromosome 2 (BTA2) where either Cytosine (C) or Thymine (T) can be found.

According to another aspect of the method, there is provided a method for predicting cattle animal behavior by determining the nucleotide present at locus ARS-BFGL-NGS-111311 alone or combine with any other cattle loci. This locus can be mapped at position 51,300,416 (Btau4.0) of bovine chromosome 23 (BTA23) where either Cytosine (C), or Thymine (T) can be found.

According to another aspect of the method, there is provided a method for predicting cattle animal behavior by determining the nucleotide present at locus BTB-01570493 alone or combine with any other cattle loci. This locus can be mapped at position 25,395,611 (Btau4.0) of bovine chromosome 8 (BTA8) where either Guanidine (G) or Adenosine (A) can be found.

According to another aspect of the method, there is provided a method for predicting cattle animal behavior by determining the nucleotide present at locus ARS-BFGL-NGS-104914 alone or combine with any other cattle loci. This locus can be mapped at position 125,588,038 (Btau4.0) of bovine chromosome 5 (BTA5) where either Thymine (T) or Cytosine (C) can be found.

According to another aspect of the method, there is provided a method for predicting cattle animal behavior by determining the nucleotide present at locus BTA-114011-no-rs alone or combine with any other cattle loci. This locus can be mapped at position 125,911,737 (Btau4.0) of bovine chromosome 1 (BTA1) where either Adenosine (A) or Guanidine (G) can be found.

According to another aspect of the method, there is provided a method for predicting cattle animal behavior by determining the nucleotide present at locus ARS-BFGL-NGS-23375 alone or combine with any other cattle loci. This locus can be mapped at position 40,238,627 (Btau4.0) of bovine chromosome 24 (BTA24) where either Adenosine (A) or Guanidine (G) can be found.

According to another aspect of the method, there is provided a method for predicting cattle animal behavior by determining the nucleotide present at locus ARS-BFGL-NGS-78666 alone or combine with any other cattle loci. This locus can be mapped at position 136,573,912 (Btau4.0) of bovine chromosome 1 (BTA1) where either Cytosine (C) or Thymine (T) can be found.

According to another aspect of the method, there is provided a method for predicting cattle animal behavior by determining the nucleotide present at locus BTB-01087838 alone or combine with any other cattle loci. This locus can be mapped at position 89,620,818 (Btau4.0) of bovine chromosome 10 (BTA10) where either Adenosine (A) or Guanidine (G) can be found.

According to another aspect of the method, there is provided a method for predicting cattle animal behavior by determining the nucleotide present at locus Hapmap31564-BTC-007633 alone or combine with any other cattle loci. This locus can be mapped at position 7,998,737 (Btau4.0) of bovine chromosome 14 (BTA14) where either Adenosine (A) or Guanidine (G) can be found.

According to another aspect of the method, there is provided a method for predicting cattle animal behavior by determining the nucleotide present at locus Hapmap50402-BTA-58146 alone or combine with any other cattle loci. This locus can be mapped at position 42,593,193 (Btau4.0) of bovine chromosome 24 (BTA24) where either Guanidine (G) or Adenosine (A) can be found.

According to another aspect of the method, there is provided a method for predicting cattle animal behavior by determining the nucleotide present at locus ARS-BFGL-BAC-46971 alone or combine with any other cattle loci. This locus can be mapped at position 35,184,932 (Btau4.0) of bovine chromosome 25 (BTA25) where either Thymine (T) or Cytosine (C) can be found.

The following examples are provided to aid the understanding of the present disclosure, the true scope of which is set forth in the claims. It is understood that modifications can be made in the procedures set forth without departing from the spirit or scope of the methods defined herein.

### EXAMPLES

### Example 1

### Animal Selection

Initially, a small group study of 1,189 cattle from the farm Jacarezinho (Araçatuba, SP—Brazil) were evaluated. All these animals were pure contemporary Nellore breed, having similar ages and were submitted to similar nutritional programs. The parameters measured in these animals behavior after release from the crush (animals held for 5-10 minutes, blood samples taken, and then released) accounting were flight speed (FS) and plasma cortisol levels. The term “flight speed” or “FS” as used herein is defined as the time to run 1.7 meters detected with sensors and measured in milliseconds.

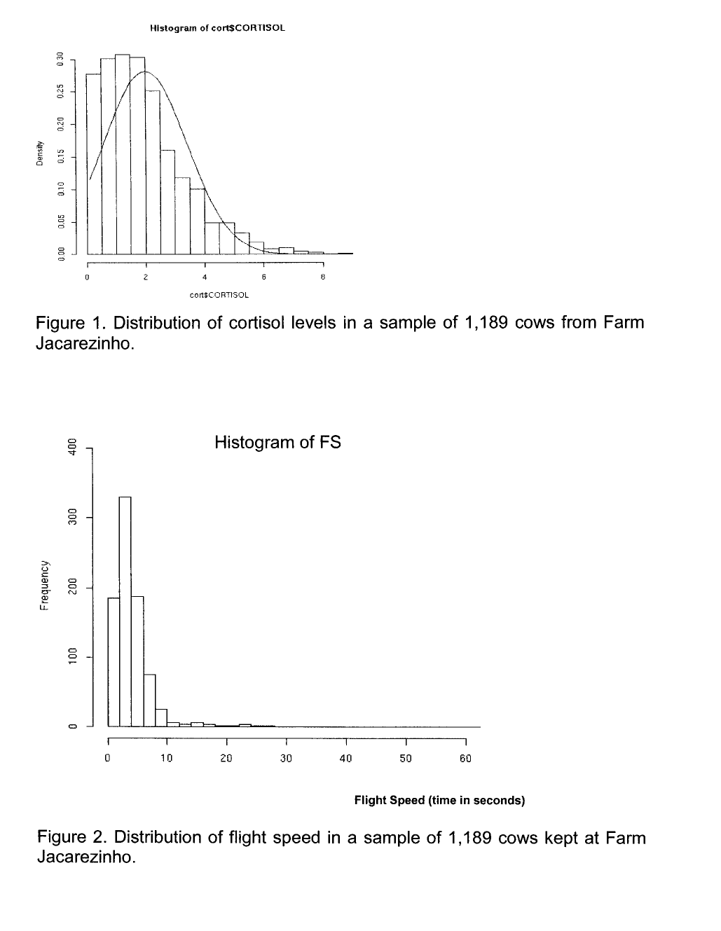

Cortisol levels in 1,189 cattle were analyzed. As shown in FIG. 1, the cortisol levels did not show a normal distribution. On average, the cortisol levels were 1.995 mcg/dL (micrograms per deciliters), with a standard deviation of 1.417 mcg/dL. Based on the asymmetrical distribution of cortisol levels, two groups of animals, referred to as “inferior” and “superior” were selected for genotyping experiments. The “inferior” group of animals comprises animals with cortisol values equal or lesser than the value of the 10th percentile (0.4 mcg/dL). The “superior” group comprises animals with cortisol values equal or greater than 90th percentile (4.0 mcg/dL). The inferior group comprised 124 animals whereas the superior group had 119 animals available. A sample of 75 animals on each side is representative of polar behavior. “Polar behavior” as used herein means grouping of extreme calm or aggressive individuals.

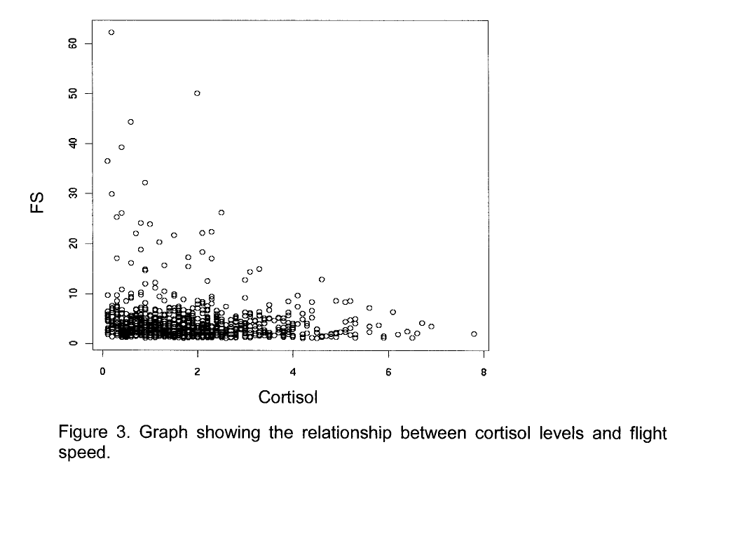

FIG. 2 shows the distribution of flight speed in a sample of 1,189 cattle. As with cortisol level, the flight speed data did not show a normal distribution pattern. Most of the animals had a flight speed of less than 5 milliseconds, and it was therefore difficult to separate the animals into extreme groups. As shown in FIG. 3, flight speed could not be correlated with cortisol levels; the R-squared value was only 0.0151.

The distribution of flight speeds was shifted to lowest flight times (or highest speed), suggesting that the animals could be subject to stress conditions during data collection. Most of the animals had low levels of cortisol, suggesting that although flight speeds were high, there may be some type of selection for lineages with more calm behavior. As it would appear that biochemical parameters such as cortisol levels would be less sensitive to handling conditions as compared to parameters such as flight speed, cortisol levels were used as a phenotypical marker.

### Example 2

### Genome-Wide Association Study (GWAS)

Genome-wide association studies (GWAS) in cattle with statistical analyses to estimate cortisol level based on genotype can be performed. The cortisol level can be associated with other bovine commercial traits which can include, but are not restricted to, body weight and carcass finishing (fat thickness and rib eye area).

Association analyses can be used as a statistical method in GWAS. The association between a given SNP and a trait can be carried out using a SNP as a categorical variable with one level for each possible genotype. To assess the association between the phenotype Y (quantitative or binary) and a SNP, a general linear model (GLM) was used:

Yᵢ=a+bXᵢ+Zᵢ

where a is the intercept, bXᵢ is the iₜₕ subject's genotype score for a given marker. The term Zᵢ was used to adjust the model by confounders variables denoted by Z.

For each genetic model, the mean differences for quantitative traits can be determined. Confidence intervals can also be computed using the variance estimated for each parameter. When the additive model was used, Xᵢ indicated the number of minor alleles of iₜₕ subjects. In the case of the dominant model, Xᵢ denoted, with coded values 1 and 0, whether the iₜₕ subject has at least one minor allele. In a similar way, for the recessive model, Xᵢ was codified as 1 and 0 depending on whether the iₜₕ subject had two minor alleles.

Genetic model selection can be used as a statistical method in GWAS. The statistical significance of a given SNP can be tested by comparing the effect of the polymorphism with the null model using the likelihood ratio test (LRT):

LRT=2(log Likₙᵤₗₗ−log Lik_{dominant})

LRT=2(log Likₙᵤₗₗ−log Lik_{recessive})

LRT=2(log Likₙᵤₗₗ−log Lik_{log-additive})

where Lik stands for likelihood.

The choice of the correct model of inheritance can be performed using the Akaike information criteria (AIC):

AIC=−2 log Lik+2q

where q denotes the number of parameters for the fitted model.

Multiple testing can also be performed. The traditional family wise error rate, as understood in the art, in procedures such as Bonferroni correction are too conservative when analyzing a large of number of events, such as is done in genome wide association experiments. Therefore, to compute the significant thresholds for the p-values originating from multiple association tests, the false discovery rate (FDR) method can be used (Benjamini, Y., and Hochberg Y. (1995) “Controlling the false discovery rate: a practical and powerful approach to multiple testing”. Journal of the Royal Statistical Society. Series B, 57, 289-300, which is herein incorporated by reference). Because of the reduced sample size, the level of significance can be defined based on chromosome-wise type 1 errors. As such, the individual p-values can be sorted from smallest to largest being the i-th smallest p-value (in the i-th row) by p₍ᵢ₎, for each i between 1 and m, being m the number of tested SNPs in a chromosome. Starting from the largest p-value p₍ₘ₎, p₍ᵢ₎ was compared with 0.051*i/m, and continued therefrom as long as p₍ᵢ₎>0.05*i/m. k was defined as the first instance when p₍ₖ₎ was less than or equal to 0.05*k/m. Hypotheses corresponding to the smallest k p-values were rejected.

### Example 3

### GWAS Results

The genotypes of 111 brazilian Nelore bulls were analyzed for 48,528 SNPs. 14,416 of these SNPs were monomorphic in the analyzed population. The remaining 34,112 SNPs were subject to association studies based on the single trait analysis described herein. In this analysis, cortisol levels can be used as a dependent variable and effects of genotypes can be assessed in three genetic models: dominant, recessive and log-additive.

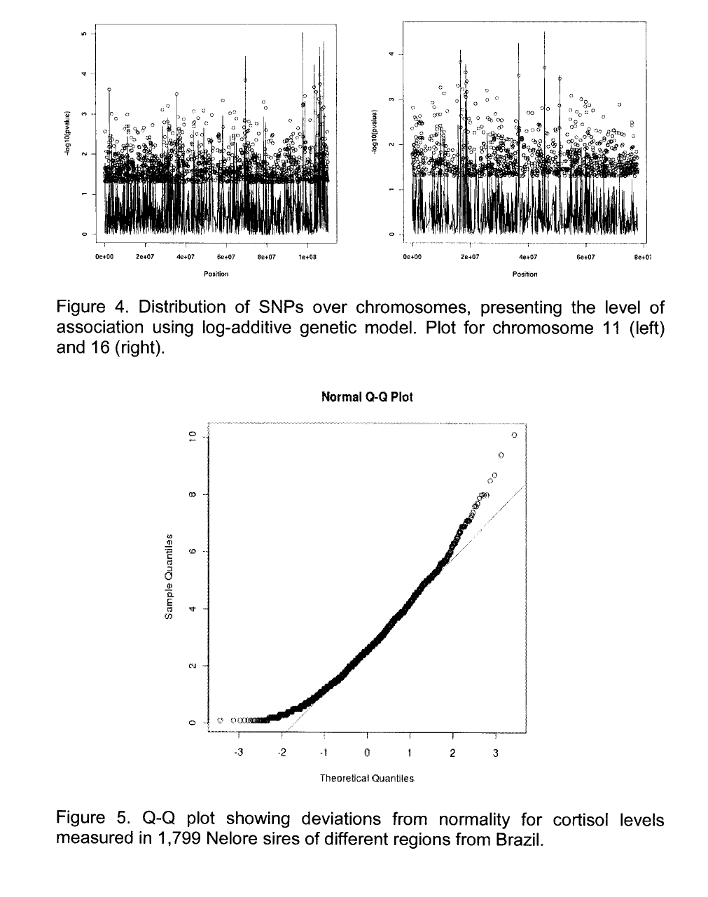

After the application of the multiple testing threshold, 21 polymorphisms were revealed to be associated with stress tolerance at the chromosome-wise level (p<0.05). Table I summarizes these results. Fourteen polymorphisms were significant in both dominant and log additive models. Seven polymorphisms are localized at chromosome 11 and seven at chromosome 16, thereby defining potential quantitative trait loci in these autosomes. FIG. 4 shows the distribution of SNPs over these respective chromosomes and the level of association using log-additive genetic model (the −log 10(p-value) for the association tests).

### Example 4

### Validation of GWAS Results Using Animals from Higher Genetic Diversity Populations

As described, 111 brazilian Nelore bulls were genotyped for 48,528 SNPs. Of these, 14,416 were monomorphic in the analyzed population. The remaining 34,112 SNP were submitted to association studies based on single trait analyzes described herein. As such, cortisol level can be used as a dependent variable and effects of genotypes can be assessed in four genetic models: codominant, dominant, recessive, and log-additive. FIG. 3 shows the distribution of SNPs over the respective chromosome and the −log 10(p-value) for the association tests. After the application of a multiple testing threshold, 21 polymorphisms were revealed to be associated to stress tolerance at the chromosome-wise level (p<0.05). This initial associated set was used in a larger association study as described below.

A population sampling can be performed, for example, a sample of 1,799 specimens can be used. All of the samples were intact males. These sires originated from 11 breeders from four different Brazilian regions. The sires from the different regions were chosen to explore diversity of samples in terms of cattle handling, genetic background and influence of weather on behaviour.

The evaluation of cortisol levels was carried out in plasma samples. Cortisol levels did not display a normally distribution, as shown in FIG. 5. This behavior can be expected given the multifactoral nature of hormone production.

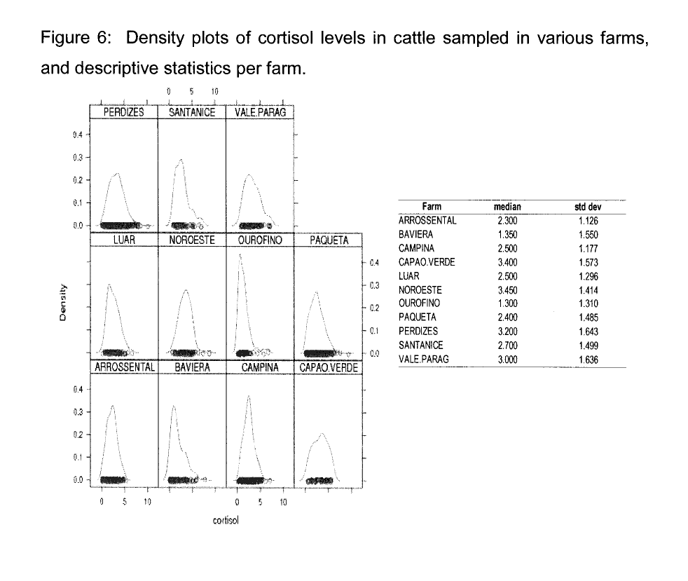

To understand the influence of cattle handling in cortisol levels, the variance of cortisol level in each farm can be analyzed. As can be seen from FIG. 6, differences in the cortisol level distribution in animals from different origins were observed. Density in FIG. 6 can be determined as is known in the art and can demonstrate the percentile of animals with a measured cortisol level. Cortisol can be measured in units of micrograms per deciliters (x axis, varying from 0 to 10). This heterogeneity can be associated with cattle handling, and can also reflect differences in founder groups.

### Validation Results

Forty-six loci were genotyped based on the results of GWAS. The results shown in Table I indicate that the SNPlex™ strategy by Applied Biosystems™ adopted for the validation step was successful, as the SNPs presented could be genotyped. Most SNPs had an allele frequency of <90% and 17 of the SNPs were in Hardy-Weinberg equilibrium. Initially, single marker association was performed using the general linear model method described above to measure the relationships between genotype and phenotype. Table II shows the mean and standard errors for each genotype, the mean difference, and its 95% confidence interval with respect to the most frequent homozygous genotype. Table III summarizes the results for the SNPs showing a significant association (p<=0.05). Despite the fact that some markers showed a strong correlation with high levels of cortisol, in most cases, the correlation between cortisol and genotype was low (highest R-squared=0.05). This result can be interpreted to be in agreement with the complex nature of a trait such as cortisol levels.

The additive and epistatic effects among markers were evaluated using a mixed model where the effect of a second marker is treated as random. Tables IV and V show these results. Table V summarizes interactions by F-statistics, while Table VI shows all data from linear model, including regression coefficients. Table VI presents exhaustive data from marker interactions evaluated using mixed models. As seen in Tables III, IV, V, and VI, there are genetic interactions between some markers as would be apparent to one skilled in the art. The filtered data suggests that is possible to build a model based on markers ARSBFGLNGS97162, ARSBFGLNGS102860, ARSBFGLNGS30157, ARSBFGLNGS68110 and HAPMAP53129RS29022984. A person skilled in the art would understand that this model can explain, at a genetic level, the increase of cortisol level in studied population.

Although the foregoing method and assays have been described in some detail by way of illustration and example for purposes of clarity of understanding, it is readily apparent to those of ordinary skill in the art in light of the teachings that certain changes and modifications may be made thereto without departing from the spirit or scope of the appended claims. Unless defined otherwise all technical and scientific terms used herein have the same meaning as commonly understood to one of ordinary skill and the art to which this invention belongs. In addition, the terms and expressions used in this specification have been used herein as terms of description and not of limitation, and there is no intention in the use of such terms and expressions of excluding equivalents of the features shown and described or portions thereof, it being recognized that the scope of the invention is defined and limited only by the appended claims.

TABLE I
General information for significant SNPs associated to stress
tolerance at chromosome-wise level (p < 0.05). Values for allele frequency and
Hardy-Weinberg statistics are presented.

| | | | P-value | P-value Log- | | | | |
|---|---|---|---|---|---|---|---|---|
| SNP | Chr | Position | Dominant | additive | Alleles | Major allele freq | HWE | Missing (%) |
| ARSBFGLNGS82206 | 1 | 130073476 | | 2.15E−005 | G/A | 70.70 | 0.36 | 0.0 |
| BFGLNGS114897 | 11 | 69718191 | 9.60E−005 | 3.59E−005 | G/A | 79.70 | 0.77 | 0.0 |
| Hapmap53129rs29022984 | 11 | 97865486 | 2.00E−005 | 9.20E−006 | C/T | 78.40 | 0.78 | 0.0 |
| ARSBFGLNGS32646 | 11 | 103515295 | 1.44E−004 | 5.95E−005 | G/A | 80.20 | 1.00 | 0.0 |
| BFGLNGS119018 | 11 | 104533531 | | 2.66E−004 | G/A | 83.80 | 0.48 | 0.0 |
| ARSBFGLNGS12135 | 11 | 106208941 | 1.30E−005 | 2.15E−005 | G/A | 90.30 | 0.25 | 2.7 |
| ARSBFGLNGS68110 | 11 | 106356143 | 1.00E−006 | 3.60E−005 | C/A | 67.10 | 0.67 | 0.0 |
| ARSBFGLNGS30157 | 11 | 108365497 | 5.00E−006 | 1.54E−005 | G/A | 87.70 | 0.66 | 0.9 |
| BTA98582nors | 15 | 104533531 | 3.40E−005 | | C/T | 50.90 | 0.13 | 0.0 |
| Hapmap50501BTA91866 | 16 | 16697956 | 1.00E−005 | 7.99E−005 | T/C | 77.00 | 0.79 | 0.0 |
| ARSBFGLNGS55834 | 16 | 18500741 | 2.85E−004 | 1.69E−004 | C/T | 79.30 | 0.15 | 0.0 |
| Hapmap49592BTA38891 | 16 | 36808658 | | 5.65E−005 | G/A | 80.60 | 0.55 | 0.0 |
| ARSBFGLNGS102860 | 16 | 36875751 | | 1.73E−004 | A/G | 79.30 | 1.00 | 0.0 |
| ARSBFGLNGS100843 | 16 | 45768093 | 2.40E−005 | 2.84E−005 | C/T | 88.70 | 1.00 | 0.0 |
| ARSBFGLNGS43639 | 16 | 45798239 | 1.56E−004 | 1.56E−004 | G/A | 90.10 | — | 0.0 |
| ARSBFGLNGS97162 | 16 | 51027090 | | 2.88E−004 | C/T | 88.60 | 1.00 | 0.9 |
| ARSBFGLBAC2384 | 19 | 31838305 | 1.00E−006 | 5.90E−006 | G/T | 56.80 | 0.85 | 0.0 |
| BFGLNGS114602 | 20 | 2011969 | 1.30E−005 | 1.86E−005 | T/C | 69.40 | 0.66 | 0.0 |
| ARSBFGLNGS10830 | 21 | 14303664 | 2.90E−005 | | C/G | 58.60 | 1.00 | 0.0 |
| ARSBFGLBAC35732 | 22 | 37243030 | 1.70E−005 | 1.78E−005 | A/G | 85.50 | 0.46 | 0.9 |
| BTB00000725 | 27 | 19405584 | | 8.50E−006 | A/G | 72.00 | 0.81 | 1.8 |
| Hapmap32414BTA65998 | 28 | 102319 | 6.00E−06 | | C/T | 61.30 | 0.69 | 0.0 |

TABLE II
Mean and standard errors for each genotype, mean difference and
its 95% confidence interval with respect to the most frequent
homozygous genotype.

| | | | Std. | Mean | | |
|---|---|---|---|---|---|---|
| | N | Mean | error | difference | CI(95) | |
| ARSBFGLNGS82206 | | | | | | |
| G/G | 53 | 3.62 | 0.31 | 0 | | |
| A/G-A/A | 58 | 1.82 | 0.30 | −1.79 | −2.64 | −0.95 |
| BFGLNGS114897 | | | | | | |
| G/G | 71 | 2.04 | 0.28 | 0 | | |
| A/G-A/A | 40 | 3.81 | 0.35 | 1.77 | 0.88 | 2.66 |
| Hapmap53129rs29022984 | | | | | | |
| C/C | 67 | 3.42 | 0.29 | 0 | | |
| T/C-T/T | 44 | 1.54 | 0.32 | −1.88 | −2.74 | −1.02 |
| ARSBFGLNGS32646 | | | | | | |
| G/G | 71 | 3.30 | 0.28 | 0 | | |
| A/G-A/A | 40 | 1.57 | 0.34 | −1.73 | −2.62 | −0.84 |
| BFGLNGS119018 | | | | | | |
| G/G | 79 | 3.16 | 0.27 | 0 | | |
| A/G-A/A | 32 | 1.48 | 0.38 | −1.68 | −2.63 | −0.72 |
| ARSBFGLNGS12135 | | | | | | |
| G/G | 89 | 2.17 | 0.24 | 0 | | |
| A/G-A/A | 19 | 4.64 | 0.50 | 2.47 | 1.36 | 3.58 |
| ARSBFGLNGS68110 | | | | | | |
| C/C | 51 | 3.78 | 0.31 | 0 | | |
| A/C-A/A | 60 | 1.74 | 0.29 | −2.04 | −2.87 | −1.21 |
| ARSBFGLNGS30157 | | | | | | |
| G/G | 85 | 2.17 | 0.26 | 0 | | |
| A/G-A/A | 25 | 4.50 | 0.36 | 2.32 | 1.32 | 3.33 |
| BTA98582nors | | | | | | |
| C/C | 33 | 1.30 | 0.35 | 0 | | |
| T/C-T/T | 78 | 3.26 | 0.27 | 1.96 | 1.03 | 2.89 |
| Hapmap50501BTA91866 | | | | | | |
| T/T | 65 | 3.47 | 0.30 | 0 | | |
| T/C-C/C | 46 | 1.55 | 0.31 | −1.92 | −2.77 | −1.07 |
| ARSBFGLNGS55834 | | | | | | |
| C/C | 67 | 3.32 | 0.30 | 0 | | |
| T/C-T/T | 44 | 1.69 | 0.32 | −1.63 | −2.51 | −0.75 |
| Hapmap49592BTA38891 | | | | | | |
| G/G | 73 | 3.28 | 0.28 | 0 | | |
| A/G-A/A | 38 | 1.53 | 0.35 | −1.75 | −2.65 | −0.84 |
| ARSBFGLNGS102860 | | | | | | |
| A/A | 70 | 3.26 | 0.28 | 0 | | |
| A/G-G/G | 41 | 1.68 | 0.35 | −1.58 | −2.48 | −0.68 |
| ARSBFGLNGS100843 | | | | | | |
| C/C | 87 | 3.16 | 0.26 | 0 | | |
| T/C-T/T | 24 | 0.94 | 0.34 | −2.22 | −3.24 | −1.19 |
| ARSBFGLNGS43639 | | | | | | |
| G/G | 89 | 3.09 | 0.26 | 0 | | |
| A/G | 22 | 1.01 | 0.37 | −2.08 | −3.15 | −1.00 |
| ARSBFGLNGS97162 | | | | | | |
| C/C | 86 | 3.07 | 0.26 | 0 | | |
| T/C-T/T | 24 | 1.14 | 0.36 | −1.94 | −2.98 | −0.89 |
| ARSBFGLBAC2384 | | | | | | |
| G/G | 35 | 4.19 | 0.37 | 0 | | |
| T/G-T/T | 76 | 1.98 | 0.26 | −2.2 | −3.09 | −1.31 |
| BFGLNGS114602 | | | | | | |
| T/T | 52 | 3.68 | 0.32 | 0 | | |
| T/C-C/C | 59 | 1.80 | 0.29 | −1.88 | −2.72 | −1.03 |
| ARSBFGLNGS10830 | | | | | | |
| C/C | 38 | 1.43 | 0.36 | 0 | | |
| C/G-G/G | 73 | 3.33 | 0.27 | 1.9 | 1.01 | 2.8 |
| ARSBFGLBAC35732 | | | | | | |
| A/A | 79 | 2.12 | 0.27 | 0 | | |
| A/G-G/G | 31 | 4.18 | 0.33 | 2.06 | 1.12 | 3 |
| BTB00000725 | | | | | | |
| A/A | 57 | 1.92 | 0.29 | 0 | | |
| A/G-G/G | 52 | 3.61 | 0.33 | 1.69 | 0.83 | 2.55 |
| Hapmap32414BTA65998 | | | | | | |
| C/C | 43 | 1.48 | 0.31 | 0 | | |
| T/C-T/T | 68 | 3.45 | 0.29 | 1.99 | 1.13 | 2.85 |

TABLE III
General information for SNPs associated with stress tolerance (p < 0.05).
Values for allele frequency and Hardy-Weinberg statistics are presented.

| SNP | Alleles | Major allele frequenecy | HWE p-value | Missing |
|---|---|---|---|---|
| ARSBFGLBAC46971 | T/C | 82.3 | 0.0000000 | 28.5 |
| ARSBFGLNGS102860 | T/C | 79.6 | 0.3761790 | 2.8 |
| BFGLNGS119018 | G/A | 86.0 | 0.6187500 | 3.3 |
| HAPMAP26724BTA152272 | T/C | 93.3 | 0.8489040 | 2.8 |
| ARSBFGLNGS27655 | A/G | 73.3 | 0.0000000 | 4.2 |
| ARSBFGLNGS82206 | G/A | 76.0 | 0.7385690 | 6.3 |
| BFGLNGS112731 | G/A | 55.4 | 0.0000000 | 7.6 |
| ARSBFGLNGS12135 | T/C | 60.7 | 0.2082270 | 38.5 |
| HAPMAP42580BTA54259 | G/A | 74.7 | 0.2540140 | 2.9 |
| ARSBFGLBAC20850 | T/C | 92.6 | 0.7267820 | 4.4 |
| ARSBFGLNGS100843 | G/A | 92.1 | 0.0936910 | 5.3 |
| BTB01548453 | G/T | 61.1 | 0.8013320 | 2.8 |
| INRA453 | A/G | 75.9 | 0.0083940 | 4.4 |
| BTB00186413 | A/G | 89.0 | 0.0005960 | 7.4 |
| ARSBFGLNGS97162 | C/T | 62.8 | 0.0000000 | 9.2 |
| Hapmap42294BTA69421 | G/A | 58.7 | 0.4593730 | 2.9 |
| ARSBFGLBAC2384 | G/T | 65.8 | 0.0000250 | 3.3 |
| ARSBFGLNGS10830 | G/C | 62.5 | 0.3568340 | 3.5 |
| BTB01553536 | T/C | 60.6 | 0.0000000 | 5.6 |
| UAIFASA7842 | G/T | 91.9 | 0.0001540 | 17.2 |
| HAPMAP32414BTA65998 | C/T | 62.5 | 0.0238830 | 4.1 |
| BFGLNGS114602 | A/G | 78.9 | 0.7736110 | 2.7 |
| BTB01944037 | G/A | 81.6 | 0.0800710 | 2.7 |
| BTB00086583 | C/T | 99.1 | 0.0000040 | 8.5 |
| ARSBFGLNGS43639 | C/T | 94.1 | 0.8294910 | 3.2 |
| BFGLNGS111311 | C/T | 56.4 | 0.1432650 | 3.6 |
| HAPMAP53129RS29022984 | G/A | 87.5 | 0.1234170 | 4.3 |
| BTB01570493 | G/A | 93.3 | 0.0107510 | 3.2 |
| ARSBFGLNGS104914 | T/C | 68.5 | 0.0000000 | 59.1 |
| BTA.114011NORS | A/G | 93.4 | 0.8445240 | 3.4 |
| ARSBFGLNGS23375 | A/G | 57.2 | 0.7295910 | 4.9 |
| ARSBFGLNGS68110 | C/A | 61.9 | 0.0158450 | 5.1 |
| ARSBFGLNGS55834 | G/A | 81.8 | 0.4162300 | 4.9 |
| ARSBFGLNGS78666 | C/T | 73.3 | 0.0115640 | 4.4 |
| BTB01087838 | A/G | 91.6 | 0.2137320 | 2.9 |
| HAPMAP31564BTC007633 | A/G | 92.7 | 0.7242100 | 4.2 |
| BFGLNGS114897 | G/A | 79.6 | 0.0000000 | 9.0 |
| BTB00000725 | T/C | 69.6 | 0.4617660 | 3.4 |
| ARSBFGLNGS32646 | G/A | 78.2 | 0.3798730 | 49.9 |
| BTA98582NORS | C/T | 53.0 | 0.5898150 | 6.7 |
| HAPMAP49592BTA38891 | C/T | 83.8 | 0.6601130 | 2.9 |
| ARSBFGLNGS30157 | G/A | 88.5 | 0.0010160 | 7.2 |
| ARSBFGLBAC35732 | T/C | 83.2 | 0.0833420 | 5.0 |
| HAPMAP50501BTA91866 | T/C | 85.9 | 0.7650620 | 3.9 |
| HAPMAP50402BTA58146 | G/A | 51.5 | 0.3388270 | 2.8 |
| HAPMAP30097BTC007678 | C/T | 91.5 | 0.1597550 | 4.2 |

TABLE IV
Mean and standard errors for each genotype, mean difference and its
95% confidence interval with respect to the most frequent homozygous genotype.
$ARSBFGLNGS102860
SNP: ARSBFGLNGS102860 adjusted by:

| | n | me | se | dif | lower | upper p-value | AIC |
|---|---|---|---|---|---|---|---|
| Codominant | | | | | | | |
| T/T | 1057 | 2.721 | 0.04658 | 0.00000 | | 0.02053 | 6181 |
| T/C | 552 | 2.806 | 0.06810 | 0.08526 | −0.07227 | 0.24279 | |
| C/C | 65 | 2.251 | 0.14069 | −0.46995 | −0.85330 | −0.08660 | |
| Dominant | | | | | | | |
| T/T | 1057 | 2.721 | 0.04658 | 0.00000 | | 0.73045 | 6186 |
| T/C-C/C | 617 | 2.747 | 0.06305 | 0.02677 | −0.12552 | 0.17905 | |
| Recessive | | | | | | | |
| T/T-T/C | 1609 | 2.750 | 0.03850 | 0.00000 | | 0.00994 | 6180 |
| C/C | 65 | 2.251 | 0.14069 | −0.49920 | −0.87874 | −0.11966 | |
| Overdominant | | | | | | | |
| T/T-C/C | 1122 | 2.693 | 0.04474 | 0.00000 | | 0.15807 | 6184 |
| T/C | 552 | 2.806 | 0.06810 | 0.11248 | −0.04370 | 0.26867 | |
| log-Additive | | | | | | | |
| 0, 1, 2 | | | −0.03886 | −0.16891 | 0.09118 | 0.55805 | 6186 |

$BFGLNGS119018
SNP: BFGLNGS119018 adjusted by:

| | n | me | se | dif | lower | upper p-value | AIC |
|---|---|---|---|---|---|---|---|
| Codominant | | | | | | | |
| G/G | 1220 | 2.778 | 0.04538 | 0.0000 | | 0.10337 | 6153 |
| G/A | 413 | 2.619 | 0.06878 | −0.1595 | −0.3307 | 0.011774 | |
| A/A | 31 | 2.432 | 0.22116 | −0.3461 | −0.8932 | 0.200983 | |
| Dominant | | | | | | | |
| G/G | 1220 | 2.778 | 0.04538 | 0.0000 | | 0.04253 | 6151 |
| G/A-A/A | 444 | 2.606 | 0.06579 | −0.1725 | −0.3392 | −0.005812 | |
| Recessive | | | | | | | |
| G/G-G/A | 1633 | 2.738 | 0.03813 | 0.0000 | | 0.27215 | 6154 |
| A/A | 31 | 2.432 | 0.22116 | −0.3058 | −0.8515 | 0.239981 | |
| Overdominant | | | | | | | |
| G/G-A/A | 1251 | 2.770 | 0.04460 | 0.0000 | | 0.08324 | 6152 |
| G/A | 413 | 2.619 | 0.06878 | −0.1509 | −0.3216 | 0.019840 | |
| log-Additive | | | | | | | |
| 0, 1, 2 | | | −0.1631 | −0.3132 | −0.012975 | 0.03322 | 6151 |

$ARSBFGLBAC20850
SNP: ARSBFGLBAC20850 adjusted by:

| | n | me | se | dif | lower | upper p-value | AIC |
|---|---|---|---|---|---|---|---|
| Codominant | | | | | | | |
| T/T | 1402 | 2.742 | 0.04142 | 0.000000 | | 0.06522 | 6087 |
| T/C | 236 | 2.663 | 0.09293 | −0.079228 | −0.2909 | 0.1325 | |
| C/C | 8 | 3.938 | 0.64252 | 1.195560 | 0.1288 | 2.2623 | |
| Dominant | | | | | | | |
| T/T | 1402 | 2.742 | 0.04142 | 0.000000 | | 0.72553 | 6090 |
| T/C-C/C | 244 | 2.705 | 0.09317 | −0.037432 | −0.2464 | 0.1715 | |
| Recessive | | | | | | | |
| T/T-T/C | 1638 | 2.731 | 0.03789 | 0.000000 | | 0.02650 | 6086 |
| C/C | 8 | 3.938 | 0.64252 | 1.206975 | 0.1408 | 2.2731 | |
| Overdominant | | | | | | | |
| T/T-C/C | 1410 | 2.749 | 0.04139 | 0.000000 | | 0.42617 | 6090 |
| T/C | 236 | 2.663 | 0.09293 | −0.086012 | −0.2979 | 0.1258 | |
| log-Additive | | | | | | | |
| 0, 1, 2 | | | 0.007974 | −0.1909 | 0.2069 | 0.93737 | 6090 |

$ARSBFGLNGS100843
SNP: ARSBFGLNGS100843 adjusted by:

| | n | me | se | dif | lower | upper p-value | AIC |
|---|---|---|---|---|---|---|---|
| Codominant | | | | | | | |
| G/G | 1384 | 2.694 | 0.04156 | 0.0000 | | 0.09967 | 6026 |
| G/A | 230 | 2.882 | 0.09681 | 0.1874 | −0.026669 | 0.4016 | |
| A/A | 16 | 3.219 | 0.34955 | 0.5245 | −0.231620 | 1.2805 | |
| Dominant | | | | | | | |
| G/G | 1384 | 2.694 | 0.04156 | 0.0000 | | 0.04856 | 6025 |
| G/A-A/A | 246 | 2.904 | 0.09331 | 0.2094 | 0.001324 | 0.4174 | |
| Recessive | | | | | | | |
| G/G-G/A | 1614 | 2.721 | 0.03824 | 0.0000 | | 0.19685 | 6027 |
| A/A | 16 | 3.219 | 0.34955 | 0.4977 | −0.258167 | 1.2537 | |
| Overdominant | | | | | | | |
| G/G-A/A | 1400 | 2.700 | 0.04130 | 0.0000 | | 0.09653 | 6026 |
| G/A | 230 | 2.882 | 0.09681 | 0.1815 | −0.032545 | 0.3955 | |
| log-Additive | | | | | | | |
| 0, 1, 2 | | | 0.2049 | 0.015508 | 0.3943 | 0.03397 | 6024 |

$ARSBFGLNGS97162
SNP: ARSBFGLNGS97162 adjusted by:

| | n | me | se | dif | lower | upper p-value | AIC |
|---|---|---|---|---|---|---|---|
| Codominant | | | | | | | |
| C/C | 430 | 2.865 | 0.07214 | 0.00000 | | 0.021296 | 5769 |
| C/T | 1113 | 2.625 | 0.04661 | −0.24059 | −0.4120 | −0.06914 | |
| T/T | 14 | 2.843 | 0.46181 | −0.02226 | −0.8423 | 0.79775 | |
| Dominant | | | | | | | |
| C/C | 430 | 2.865 | 0.07214 | 0.00000 | | 0.006435 | 5768 |
| C/T-T/T | 1127 | 2.627 | 0.04637 | −0.23788 | −0.4090 | −0.06677 | |
| Recessive | | | | | | | |
| C/C-C/T | 1543 | 2.692 | 0.03926 | 0.00000 | | 0.715107 | 5775 |
| T/T | 14 | 2.843 | 0.46181 | 0.15128 | −0.6611 | 0.96362 | |
| Overdominant | | | | | | | |
| C/C-T/T | 444 | 2.864 | 0.07126 | 0.00000 | | 0.005520 | 5767 |
| C/T | 1113 | 2.625 | 0.04661 | −0.23989 | −0.4093 | −0.07046 | |
| log-Additive | | | | | | | |
| 0, 1, 2 | | | −0.21613 | −0.3816 | −0.05062 | 0.010485 | 5768 |

$Hapmap42294BTA69421
SNP: Hapmap42294BTA69421 adjusted by:

| | n | me | se | dif | lower | upper p-value | AIC |
|---|---|---|---|---|---|---|---|
| Codominant | | | | | | | |
| G/G | 576 | 2.832 | 0.06768 | 0.00000 | | 0.10286 | 6176 |
| G/A | 801 | 2.691 | 0.05352 | −0.14108 | −0.3051 | 0.022927 | |
| A/A | 295 | 2.622 | 0.08058 | −0.21043 | −0.4254 | 0.004505 | |
| Dominant | | | | | | | |
| G/G | 576 | 2.832 | 0.06768 | 0.00000 | | 0.04267 | 6174 |
| G/A-A/A | 1096 | 2.673 | 0.04472 | −0.15975 | −0.3142 | −0.005274 | |
| Recessive | | | | | | | |
| G/G-G/A | 1377 | 2.750 | 0.04211 | 0.00000 | | 0.19170 | 6177 |
| A/A | 295 | 2.622 | 0.08058 | −0.12837 | −0.3211 | 0.064343 | |
| Overdominant | | | | | | | |
| G/G-A/A | 871 | 2.761 | 0.05250 | 0.00000 | | 0.35225 | 6177 |
| G/A | 801 | 2.691 | 0.05352 | −0.06981 | −0.2169 | 0.077275 | |
| log-Additive | | | | | | | |
| 0, 1, 2 | | | −0.11108 | −0.2156 | −0.006504 | 0.03735 | 6174 |

$ARSBFGLBAC2384
SNP: ARSBFGLBAC2384 adjusted by:

| | n | me | se | dif | lower | upper p-value | AIC |
|---|---|---|---|---|---|---|---|
| Codominant | | | | | | | |
| G/G | 706 | 2.841 | 0.05852 | 0.00000 | | 0.028953 | 6126 |
| G/T | 806 | 2.633 | 0.05283 | −0.20822 | −0.3624 | −0.05408 | |
| T/T | 150 | 2.695 | 0.12443 | −0.14655 | −0.4154 | 0.12228 | |
| Dominant | | | | | | | |
| G/G | 706 | 2.841 | 0.05852 | 0.00000 | | 0.008712 | 6124 |
| G/T-T/T | 956 | 2.643 | 0.04861 | −0.19854 | −0.3469 | −0.05019 | |
| Recessive | | | | | | | |
| G/G-G/T | 1512 | 2.730 | 0.03932 | 0.00000 | | 0.785793 | 6131 |
| T/T | 150 | 2.695 | 0.12443 | −0.03556 | −0.2920 | 0.22087 | |
| Overdominant | | | | | | | |
| G/G-T/T | 856 | 2.816 | 0.05297 | 0.00000 | | 0.014783 | 6125 |
| G/T | 806 | 2.633 | 0.05283 | −0.18253 | −0.3293 | −0.03577 | |
| log-Additive | | | | | | | |
| 0, 1, 2 | | | −0.12758 | −0.2432 | −0.01201 | 0.030488 | 6126 |

$BTB01553536
SNP: BTB01553536 adjusted by:

| | n | me | se | dif | lower | upper p-value | AIC |
|---|---|---|---|---|---|---|---|
| Codominant | | | | | | | |
| T/T | 658 | 2.620 | 0.05695 | 0.00000 | | 0.05686 | 6009 |
| T/C | 659 | 2.820 | 0.06260 | 0.19967 | 0.033685 | 0.3656 | |
| C/C | 307 | 2.761 | 0.08793 | 0.14070 | −0.067463 | 0.3489 | |
| Dominant | | | | | | | |
| T/T | 658 | 2.620 | 0.05695 | 0.00000 | | 0.01981 | 6007 |
| T/C-C/C | 966 | 2.801 | 0.05102 | 0.18093 | 0.028724 | 0.3331 | |
| Recessive | | | | | | | |
| T/T-T/C | 1317 | 2.720 | 0.04239 | 0.00000 | | 0.67576 | 6013 |
| C/C | 307 | 2.761 | 0.08793 | 0.04079 | −0.150353 | 0.2319 | |
| Overdominant | | | | | | | |
| T/T-C/C | 965 | 2.665 | 0.04788 | 0.00000 | | 0.04611 | 6009 |
| T/C | 659 | 2.820 | 0.06260 | 0.15490 | 0.002672 | 0.3071 | |
| log-Additive | | | | | | | |
| 0, 1, 2 | | | 0.09106 | −0.009989 | 0.1921 | 0.07736 | 6010 |

$HAPMAP53129RS29022984
SNP: HAPMAP53129RS29022984 adjusted by:

| | n | me | se | dif | lower | upper p-value | AIC |
|---|---|---|---|---|---|---|---|
| Codominant | | | | | | | |
| G/G | 1267 | 2.799 | 0.04363 | 0.0000 | | 0.0034225 | 6077 |
| G/A | 344 | 2.489 | 0.07878 | −0.3098 | −0.4924 | −0.12721 | |
| A/A | 34 | 2.588 | 0.24948 | −0.2105 | −0.7324 | 0.31138 | |
| Dominant | | | | | | | |
| G/G | 1267 | 2.799 | 0.04363 | 0.0000 | | 0.0008045 | 6075 |
| G/A-A/A | 378 | 2.498 | 0.07504 | −0.3009 | −0.4768 | −0.12490 | |
| Recessive | | | | | | | |
| G/G-G/A | 1611 | 2.733 | 0.03833 | 0.0000 | | 0.5878273 | 6086 |
| A/A | 34 | 2.588 | 0.24948 | −0.1444 | −0.6664 | 0.37766 | |
| Overdominant | | | | | | | |
| G/G-A/A | 1301 | 2.793 | 0.04298 | 0.0000 | | 0.0010528 | 6076 |
| G/A | 344 | 2.489 | 0.07878 | −0.3043 | −0.4863 | −0.12224 | |
| log-Additive | | | | | | | |
| 0, 1, 2 | | | −0.2452 | −0.3999 | −0.09047 | 0.0018970 | 6077 |

$ARSBFGLNGS68110
SNP: ARSBFGLNGS68110 adjusted by:

| | n | me | se | dif | lower | upper p-value | AIC |
|---|---|---|---|---|---|---|---|
| Codominant | | | | | | | |
| C/C | 658 | 3.057 | 0.06489 | 0.0000 | | 8.430e−12 | 5992 |
| C/A | 714 | 2.534 | 0.05342 | −0.5231 | −0.6837 | −0.3625 | |
| A/A | 259 | 2.456 | 0.08388 | −0.6011 | −0.8191 | −0.3831 | |
| Dominant | | | | | | | |
| C/C | 658 | 3.057 | 0.06489 | 0.0000 | | 1.185e−12 | 5990 |
| C/A-A/A | 973 | 2.513 | 0.04511 | −0.5438 | −0.6938 | −0.3939 | |
| Recessive | | | | | | | |
| C/C-C/A | 1372 | 2.784 | 0.04231 | 0.0000 | | 1.561e−03 | 6030 |
| A/A | 259 | 2.456 | 0.08388 | −0.3289 | −0.5327 | −0.1251 | |
| Overdominant | | | | | | | |
| C/C-A/A | 917 | 2.887 | 0.05298 | 0.0000 | | 3.684e−06 | 6018 |
| C/A | 714 | 2.534 | 0.05342 | −0.3533 | −0.5029 | −0.2037 | |
| log-Additive | | | | | | | |
| 0, 1, 2 | | | −0.3480 | −0.4520 | −0.2439 | 5.547e−11 | 5997 |

$HAPMAP49592BTA38891
SNP: HAPMAP49592BTA38891 adjusted by:

| | n | me | se | dif | lower | upper p-value | AIC |
|---|---|---|---|---|---|---|---|
| Codominant | | | | | | | |
| C/C | 1169 | 2.726 | 0.04496 | 0.000000 | | 0.09554 | 6174 |
| C/T | 459 | 2.780 | 0.07239 | 0.054169 | −0.11128 | 0.219623 | |
| T/T | 43 | 2.251 | 0.17557 | −0.474842 | −0.94126 | −0.008427 | |
| Dominant | | | | | | | |
| C/C | 1169 | 2.726 | 0.04496 | 0.000000 | | 0.91386 | 6177 |
| C/T-T/T | 502 | 2.735 | 0.06816 | 0.008855 | −0.15161 | 0.169317 | |
| Recessive | | | | | | | |
| C/C-C/T | 1628 | 2.741 | 0.03819 | 0.000000 | | 0.03842 | 6172 |
| T/T | 43 | 2.251 | 0.17557 | −0.490115 | −0.95411 | −0.026120 | |
| Overdominant | | | | | | | |
| C/C-T/T | 1212 | 2.709 | 0.04387 | 0.000000 | | 0.39826 | 6176 |
| C/T | 459 | 2.780 | 0.07239 | 0.071016 | −0.09376 | 0.235787 | |
| log-Additive | | | | | | | |
| 0, 1, 2 | | | −0.038440 | −0.17967 | 0.102794 | 0.59373 | 6176 |

$ARSBFGLNGS30157
SNP: ARSBFGLNGS30157 adjusted by:

| | n | me | se | dif | lower | upper p-value | AIC |
|---|---|---|---|---|---|---|---|
| Codominant | | | | | | | |
| G/G | 1240 | 2.652 | 0.04323 | 0.0000 | | 0.007884 | 5893 |
| G/A | 344 | 2.895 | 0.08532 | 0.2427 | 0.05921 | 0.4261 | |
| A/A | 9 | 3.589 | 0.54529 | 0.9368 | −0.07038 | 1.9440 | |
| Dominant | | | | | | | |
| G/G | 1240 | 2.652 | 0.04323 | 0.0000 | | 0.004968 | 5893 |
| G/A-A/A | 353 | 2.912 | 0.08437 | 0.2604 | 0.07870 | 0.4420 | |
| Recessive | | | | | | | |
| G/G-G/A | 1584 | 2.705 | 0.03865 | 0.0000 | | 0.085666 | 5898 |
| A/A | 9 | 3.589 | 0.54529 | 0.8841 | −0.12410 | 1.8923 | |
| Overdominant | | | | | | | |
| G/G-A/A | 1249 | 2.659 | 0.04314 | 0.0000 | | 0.011717 | 5894 |
| G/A | 344 | 2.895 | 0.08532 | 0.2359 | 0.05247 | 0.4194 | |
| log-Additive | | | | | | | |
| 0, 1, 2 | | | 0.2669 | 0.09240 | 0.4413 | 0.002717 | 5892 |

$HAPMAP30097BTC007678
SNP: HAPMAP30097BTC007678 adjusted by:

| | n | me | se | dif | lower | upper p-value | AIC |
|---|---|---|---|---|---|---|---|
| Codominant | | | | | | | |
| C/C | 1378 | 2.724 | 0.04137 | 0.0000000 | | 0.06756 | 6100 |
| C/T | 254 | 2.734 | 0.09626 | 0.0101591 | −0.19538 | 0.2157 | |
| T/T | 17 | 3.594 | 0.37861 | 0.8700248 | 0.13550 | 1.6046 | |
| Dominant | | | | | | | |
| C/C | 1378 | 2.724 | 0.04137 | 0.0000000 | | 0.53044 | 6103 |
| C/T-T/T | 271 | 2.788 | 0.09397 | 0.0640990 | −0.13616 | 0.2644 | |
| Recessive | | | | | | | |
| C/C-C/T | 1632 | 2.726 | 0.03800 | 0.0000000 | | 0.02033 | 6098 |
| T/T | 17 | 3.594 | 0.37861 | 0.8684436 | 0.13483 | 1.6021 | |
| Overdominant | | | | | | | |
| C/C-T/T | 1395 | 2.735 | 0.04119 | 0.0000000 | | 0.99663 | 6103 |
| C/T | 254 | 2.734 | 0.09626 | −0.0004434 | −0.20606 | 0.2052 | |
| log-Additive | | | | | | | |
| 0, 1, 2 | | | 0.1072041 | −0.07558 | 0.2900 | 0.25033 | 6102 |

TABLE V
F-statistics for interactions between associated markers

| | F-test | P-value | F-test | P-value | F-test |
|---|---|---|---|---|---|
| | ARSBFGLNG597162 | ARSBFGLNG597162 | ARSBFGLBAC2384 | ARSBFGLBAC2384 | Hapmap42294BTA89421 |
| S97162 | 2.8523 | 0.0525 | 4.2979 | 0.0138 | 4.3045 |
| [symbol] 2384 | 0.6905 | 0.5015 | 2.2884 | 0.1018 | 2.2882 |
| BTA69421 | 3.1747 | 0.0421 | 2.3426 | 0.0964 | 2.3425 |
| S102860 | 2.8710 | 0.0570 | 3.1199 | 0.0445 | 2.9349 |
| [symbol] 018 | 0.0209 | 0.9794 | 0.8045 | 0.4475 | 1.6987 |
| | 2.2270 | 0.1082 | 2.7235 | 0.0660 | 0.2900 |
| [symbol] 2BTA38891 | 2.0154 | 0.1336 | 2.2858 | 0.1021 | 2.3398 |
| S30157 | 3.7285 | 0.0243 | 4.5715 | 0.0105 | 4.9881 |
| [symbol] 20850 | 2.0694 | 0.1266 | 1.6894 | 0.1850 | 1.8516 |
| S100843 | 2.8501 | 0.0582 | 2.5658 | 0.0772 | 2.3825 |
| S68110 | 2.6726 | NaN | 20.7953 | <.0001 | 22.4585 |
| [symbol] RS29022984 | 2.7366 | 0.0651 | 0.6312 | NaN | 5.1280 |
| [symbol] 7BTC007678 | 1.4956 | 0.2245 | 1.3454 | 0.2608 | 1.5469 |

| | P-value | F-test | P-value | F-test | P-value |
|---|---|---|---|---|---|
| | Hapmap42294BTA89421 | ARSBFGLNGS102860 | ARSBFGLNGS102860 | BFGLNGS119018 | BFGLNGS119018 |
| S97162 | 0.0137 | 4.6892 | 0.0093 | 4.1036 | 0.0167 |
| [symbol] 2384 | 0.1018 | 2.4746 | 0.0845 | 0.2562 | NaN |
| BTA69421 | 0.0964 | 2.2245 | 0.1085 | 2.3530 | 0.0954 |
| S102860 | 0.0535 | 3.0700 | 0.0467 | 3.0323 | 0.0485 |
| [symbol] 018 | 0.1833 | 1.6364 | 0.1950 | 1.4237 | 0.2412 |
| | NaN | 2.7723 | 0.0628 | 2.6086 | 0.0740 |
| [symbol] 2BTA38891 | 0.0987 | 1.0824 | NaN | 2.3904 | 0.0920 |
| S30157 | 0.0069 | 4.75 [symbol] 0 | 0.0088 | 4.8471 | 0.0080 |
| [symbol] 20850 | 0.1574 | 1.8880 | 0.1517 | 1.8974 | 0.1503 |
| S100843 | 0.0927 | 2.3212 | 0.0985 | 2.5503 | 0.0784 |
| S68110 | <.0001 | 22.8358 | <.0001 | 22.8230 | <.0001 |
| [symbol] RS29022984 | 0.0060 | 5.1570 | 0.0059 | 5.0509 | 0.0065 |
| [symbol] 7BTC007678 | 0.2133 | 1.5747 | 0.2074 | 1.5904 | 0.2042 |

| | F-test | P-value | F-test | P-value | F-test |
|---|---|---|---|---|---|
| | BT801553536 | BT801553536 | HAPMAP49592BTA38891 | HAPMAP49592BTA38891 | ARSBFGLNG830157 |
| S97162 | 4.6120 | 0.0101 | 4.4223 | 0.0122 | 1.1522 |
| [symbol] 2384 | 2.4480 | 0.0868 | 2.2969 | 0.1007 | 1.5734 |
| BTA69421 | 2.2370 | 0.1071 | 2.4755 | 0.0845 | 2.2660 |
| S102860 | 3.0700 | 0.0467 | 3.1451 | 0.0434 | 2.9546 |
| [symbol] 018 | 1.6060 | 0.2011 | 0.1996 | NaN | 1.3392 |
| | 2.7840 | 0.0621 | 2.8636 | 0.0574 | 2.4145 |
| [symbol] 2BTA38891 | 2.4040 | 0.0907 | 2.4038 | 0.0907 | 2.4080 |
| S30157 | 4.7680 | 0.0086 | 4.7336 | 0.0089 | 5.1582 |
| [symbol] 20850 | 1.8274 | NaN | 1.8359 | 0.1598 | 1.8224 |
| S100843 | 2.3320 | 0.0975 | 2.3910 | 0.0919 | 2.5482 |
| S68110 | 22.8230 | <.0001 | 22.8230 | <.0001 | 21.4071 |
| [symbol] RS29022984 | 5.1740 | 0.0058 | 5.0020 | 0.0068 | 4.9725 |
| [symbol] 7BTC007678 | 1.5750 | 0.2075 | 1.5066 | 0.2220 | 1.5605 |

| | P-value | F-test | P-value | F-test |
|---|---|---|---|---|
| | ARSBFGLNGS30157 | ARSBFGLBAC20850 | ARSBFGLBAC20850 | ARSBFGLNGS100843 |
| S97162 | NaN | 4.5901 | 0.0103 | 4.5592 |
| [symbol] 2384 | 0.2077 | 2.4578 | 0.0860 | 2.5107 |
| BTA69421 | 0.1041 | 2.0808 | 0.1252 | 0.3225 |
| S102860 | 0.0524 | 1.1187 | NaN | 2.9291 |
| [symbol] 018 | 0.2624 | 1.7129 | 0.1807 | 1.8068 |
| | 0.0898 | 2.6573 | 0.0705 | 2.8708 |
| [symbol] 2BTA38891 | 0.0904 | 2.4040 | 0.0907 | 2.3276 |
| S30157 | 0.0059 | 4.8576 | 0.0079 | 4.7200 |
| [symbol] 20850 | 0.1620 | 1.9634 | 0.1408 | 1.8099 |
| S100843 | 0.0786 | 2.4062 | 0.0905 | 2.4201 |
| S68110 | <.0001 | 22.8588 | <.0001 | 23.4752 |
| [symbol] RS29022984 | 0.0070 | 5.1705 | 0.0058 | 5.2342 |
| [symbol] 7BTC007678 | 0.2104 | 1.6405 | 0.1942 | 1.5122 |

| | P-value | F-test | P-value | F-test |
|---|---|---|---|---|
| | ARSBFGLNGS100843 | ARSBFGLNGS68110 | ARSBFGLNGS68110 | MAP63129RS2902 [symbol] |
| S97162 | 0.0106 | 4.6120 | 0.0101 | 5.3418 |
| [symbol] 2384 | 0.0815 | 2.4480 | 0.0868 | 2.7544 |
| BTA69421 | NaN | 2.2370 | 0.1071 | 2.2399 |
| S102860 | 0.0538 | 3.0700 | 0.0467 | 3.2420 |
| [symbol] 018 | 0.1646 | 1.6060 | 0.2011 | 1.6058 |
| | 0.0570 | 2.7840 | 0.0621 | 3.0693 |
| [symbol] 2BTA38891 | 0.0979 | 2.4040 | 0.0907 | 2.4110 |
| S30157 | 0.0091 | 4.7680 | 0.0086 | 1.5309 |
| [symbol] 20850 | 0.1640 | 1.8790 | 0.1532 | 1.9438 |
| S100843 | 0.0893 | 2.3320 | 0.0975 | 2.1040 |
| S68110 | <.0001 | 22.8230 | <.0001 | 22.3869 |
| [symbol] RS29022984 | 0.0054 | 5.1740 | 0.0058 | 5.0776 |
| [symbol] 7BTC007678 | 0.2208 | 1.1322 | NaN | 1.4951 |

| | P-value | F-test | P-value |
|---|---|---|---|
| | HAPMAP53129RS29022984 | HAPMAP30097BTC007678 | HAPMAP30097B [symbol] |
| S97162 | 0.0049 | 4.8300 | 0.009 [symbol] |
| [symbol] 2384 | 0.0640 | 2.6470 | 0.0712 [symbol] |
| BTA69421 | 0.1068 | 2.2953 | 0.101 [symbol] |
| S102860 | 0.0394 | 3.1141 | 0.044 [symbol] |
| [symbol] 018 | 0.2011 | 1.6670 | 0.169 [symbol] |
| | 0.0468 | 2.7358 | 0.065 [symbol] |
| [symbol] 2BTA38891 | 0.0901 | 2.3943 | 0.091 [symbol] |
| S30157 | NaN | 4.5149 | 0.011 [symbol] |
| [symbol] 20850 | 0.1435 | 1.8209 | 0.162 [symbol] |
| S100843 | 0.1223 | 0.7828 | NaN |
| S68110 | <.0001 | 23.2575 | <.000 [symbol] |
| [symbol] RS29022984 | 0.0063 | 5.2881 | 0.005 [symbol] |
| [symbol] 7BTC007678 | 0.2246 | 1.4709 | 0.230 [symbol] |

[symbol]

indicates data missing or illegible when filed

TABLE VI
Exhaustive data from marker interactions evaluated using mixed models

ARSBFGLNGS102860 × BFGLNGS119018
Linear mixed-effects model fit by REML
Data: vm

| AIC | BIC | logLik |
|---|---|---|
| 5393.213 | 5419.613 | −2691.606 |

Random effects:
Formula: ~1|BFGLNGS119018

| | | |
|---|---|---|
| | (Intercept) | Residual |
| StdDev: | 0.08989906 | 1.536882 |

Fixed effects: list(fixed)

| | | | | | |
|---|---|---|---|---|---|
| | Value | Std. Error | DF | t-value | p-value |
| (Intercept) | 2.6282119 | 0.08182014 | 1449 | 32.12182 | 0.0000 |
| ARSBFGLNGS102860T/C | 0.1178092 | 0.08665341 | 1449 | 1.35955 | 0.1742 |
| ARSBFGLNGS102860C/C | −0.3796074 | 0.20173222 | 1449 | −1.88174 | 0.0601 |
| Correlation: | | | | | |

| | | |
|---|---|---|
| | (Intr) | ARSBFGLNGS102860T |
| ARSBFGLNGS102860T/C | −0.379 | |
| ARSBFGLNGS102860C/C | −0.159 | 0.149 |

Standardized Within-Group Residuals:

| | | | | |
|---|---|---|---|---|
| Min | Q1 | Med | Q3 | Max |
| −1.7608115 | −0.7732215 | −0.1225534 | 0.6350434 | 4.8225238 |

Number of Observations: 1454
Number of Groups: 3

| | | | | |
|---|---|---|---|---|
| | numDF | denDF | F-value | p-value |
| (Intercept) | 1 | 1449 | 1242.5797 | <.0001 |
| ARSBFGLNGS102860 | 2 | 1449 | 3.1451 | 0.0434 |

ARSBFGLNGS102860 × ARSBFGLBAC20850
Linear mixed-effects model fit by REML
Data: vm

| | | |
|---|---|---|
| AIC | BIC | logLik |
| 5394.221 | 5420.621 | −2692.110 |

Random effects:
Formula: ~1|ARSBFGLBAC20850

| | | |
|---|---|---|
| | (Intercept) | Residual |
| StdDev: | 9.498082e−05 | 1.538043 |

Fixed effects: list(fixed)

| | | | | | |
|---|---|---|---|---|---|
| | Value | Std. Error | DF | t-value | p-value |
| (Intercept) | 2.6613187 | 0.05098570 | 1449 | 52.19736 | 0.0000 |
| ARSBFGLNGS102860T/C | 0.1125402 | 0.08664508 | 1449 | 1.29886 | 0.1942 |
| ARSBFGLNGS102860C/C | −0.3822864 | 0.20187619 | 1449 | −1.89367 | 0.0585 |
| Correlation: | | | | | |

| | | |
|---|---|---|
| | (Intr) | ARSBFGLNGS102860T |
| ARSBFGLNGS102860T/C | −0.588 | |
| ARSBFGLNGS102860C/C | −0.253 | 0.149 |

Standardized Within-Group Residuals:

| | | | | |
|---|---|---|---|---|
| Min | Q1 | Med | Q3 | Max |
| −1.7384811 | −0.7550625 | −0.1048857 | 0.6103088 | 4.8364582 |

Number of Observations: 1454
Number of Groups: 3

| | | | | |
|---|---|---|---|---|
| | numDF | denDF | F-value | p-value |
| (Intercept) | 1 | 1449 | 4422.301 | <.0001 |
| ARSBFGLNGS102860 | 2 | 1449 | 3.070 | 0.0467 |

ARSBFGLNGS102860 × ARSBFGLNGS100843
Linear mixed-effects model fit by REML
Data: vm

| | | |
|---|---|---|
| AIC | BIC | logLik |
| 5392.97 | 5419.37 | −2691.485 |

Random effects:
Formula: ~1|ARSBFGLNGS100843

| | | |
|---|---|---|
| | (Intercept) | Residual |
| StdDev: | 0.1330907 | 1.536622 |

Fixed effects: list(fixed)

| | | | | | |
|---|---|---|---|---|---|
| | Value | Std. Error | DF | t-value | p-value |
| (Intercept) | 2.7397382 | 0.11010374 | 1449 | 24.883243 | 0.0000 |
| ARSBFGLNGS102860T/C | 0.1102731 | 0.08657683 | 1449 | 1.273703 | 0.2030 |
| ARSBFGLNGS102860C/C | −0.3899146 | 0.20175782 | 1449 | −1.932587 | 0.0535 |
| Correlation: | | | | | |

| | | |
|---|---|---|
| | (Intr) | ARSBFGLNGS102860T |
| ARSBFGLNGS102860T/C | −0.278 | |
| ARSBFGLNGS102860C/C | −0.131 | 0.149 |

Standardized Within-Group Residuals:

| | | | | |
|---|---|---|---|---|
| Min | Q1 | Med | Q3 | Max |
| −1.75902797 | −0.74823043 | −0.09745238 | 0.61840348 | 4.85514638 |

Number of Observations: 1454
Number of Groups: 3

| | | | | |
|---|---|---|---|---|
| | numDF | denDF | F-value | p-value |
| (Intercept) | 1 | 1449 | 685.6658 | <.0001 |
| ARSBFGLNGS102860 | 2 | 1449 | 3.1141 | 0.0447 |

ARSBFGLNGS102860 × ARSBFGLNGS97162
Linear mixed-effects model fit by REML
Data: vm

| | | |
|---|---|---|
| AIC | BIC | logLik |
| 5389.965 | 5416.365 | −2689.982 |

Random effects:
Formula: ~1|ARSBFGLNGS97162

| | | |
|---|---|---|
| | (Intercept) | Residual |
| StdDev: | 0.1638062 | 1.534647 |

Fixed effects: list(fixed)

| | | | | | |
|---|---|---|---|---|---|
| | Value | Std. Error | DF | t-value | p-value |
| (Intercept) | 2.7298471 | 0.12494765 | 1449 | 21.847927 | 0.0000 |
| ARSBFGLNGS102860T/C | 0.1220546 | 0.08654753 | 1449 | 1.410261 | 0.1587 |
| ARSBFGLNGS102860C/C | −0.3520324 | 0.20178855 | 1449 | −1.744561 | 0.0813 |
| Correlation: | | | | | |

| | | |
|---|---|---|
| | (Intr) | ARSBFGLNGS102860T |
| ARSBFGLNGS102860T/C | −0.238 | |
| ARSBFGLNGS102860C/C | −0.093 | 0.151 |

Standardized Within-Group Residuals:

| | | | | |
|---|---|---|---|---|
| Min | Q1 | Med | Q3 | Max |
| −1.76340884 | −0.73519466 | −0.08914578 | 0.63894999 | 4.88882407 |

Number of Observations: 1454
Number of Groups: 3

| | | | | |
|---|---|---|---|---|
| | numDF | denDF | F-value | p-value |
| (Intercept) | 1 | 1449 | 518.0541 | <.0001 |
| ARSBFGLNGS102860 | 2 | 1449 | 2.9546 | 0.0524 |

ARSBFGLNGS102860 × Hapmap42294BTA69421
Linear mixed-effects model fit by REML
Data: vm

| | | |
|---|---|---|
| AIC | BIC | logLik |
| 5393.73 | 5420.131 | −2691.865 |

Random effects:
Formula: ~1|Hapmap42294BTA69421

| | | |
|---|---|---|
| | (Intercept) | Residual |
| StdDev: | 0.06826616 | 1.537120 |

Fixed effects: list(fixed)

| | | | | | |
|---|---|---|---|---|---|
| | Value | Std. Error | DF | t-value | p-value |
| (Intercept) | 2.6592038 | 0.06537951 | 1449 | 40.67335 | 0.0000 |
| ARSBFGLNGS102860T/C | 0.1100717 | 0.08661331 | 1449 | 1.27084 | 0.2040 |
| ARSBFGLNGS102860C/C | −0.3735997 | 0.20198511 | 1449 | −1.84964 | 0.0646 |
| Correlation: | | | | | |

| | | |
|---|---|---|
| | (Intr) | ARSBFGLNGS102860T |
| ARSBFGLNGS102860T/C | −0.458 | |
| ARSBFGLNGS102860C/C | −0.203 | 0.148 |

Standardized Within-Group Residuals:

| | | | | |
|---|---|---|---|---|
| Min | Q1 | Med | Q3 | Max |
| −1.72329099 | −0.74088793 | −0.09032076 | 0.62530312 | 4.80627513 |

Number of Observations: 1454
Number of Groups: 3

| | | | | |
|---|---|---|---|---|
| | numDF | denDF | F-value | p-value |
| (Intercept) | 1 | 1449 | 2175.8122 | <.0001 |
| ARSBFGLNGS102860 | 2 | 1449 | 2.9291 | 0.0538 |

ARSBFGLNGS102860 × ARSBFGLBAC2384
Linear mixed-effects model fit by REML
Data: vm

| | | |
|---|---|---|
| AIC | BIC | logLik |
| 5392.642 | 5419.042 | −2691.321 |

Random effects:
Formula: ~1|ARSBFGLBAC2384

| | | |
|---|---|---|
| | (Intercept) | Residual |
| StdDev: | 0.08814476 | 1.536360 |

Fixed effects: list(fixed)

| | | | | | |
|---|---|---|---|---|---|
| | Value | Std. Error | DF | t-value | p-value |
| (Intercept) | 2.6581310 | 0.07544091 | 1449 | 35.23461 | 0.0000 |
| ARSBFGLNGS102860T/C | 0.1148791 | 0.08660462 | 1449 | 1.32648 | 0.1849 |
| ARSBFGLNGS102860C/C | −0.3739759 | 0.20171270 | 1449 | −1.85400 | 0.0639 |
| Correlation: | | | | | |

| | | |
|---|---|---|
| | (Intr) | ARSBFGLNGS102860T |
| ARSBFGLNGS102860T/C | −0.407 | |
| ARSBFGLNGS102860C/C | −0.173 | 0.149 |

Standardized Within-Group Residuals:

| | | | | |
|---|---|---|---|---|
| Min | Q1 | Med | Q3 | Max |
| −1.7860907 | −0.7850879 | −0.1006010 | 0.6318841 | 4.7975752 |

Number of Observations: 1454
Number of Groups: 3

| | | | | |
|---|---|---|---|---|
| | numDF | denDF | F-value | p-value |
| (Intercept) | 1 | 1449 | 1537.3307 | <.0001 |
| ARSBFGLNGS102860 | 2 | 1449 | 3.0323 | 0.0485 |

ARSBFGLNGS102860 × BTB01553536
Linear mixed-effects model fit by REML
Data: vm

| | | |
|---|---|---|
| AIC | BIC | logLik |
| 5392.658 | 5419.058 | −2691.329 |

Random effects:
Formula: ~1|BTB01553536

| | | |
|---|---|---|
| | (Intercept) | Residual |
| StdDev: | 0.0867402 | 1.536270 |

Fixed effects: list(fixed)

| | | | | | |
|---|---|---|---|---|---|
| | Value | Std. Error | DF | t-value | p-value |
| (Intercept) | 2.6642272 | 0.07199911 | 1449 | 37.00361 | 0.0000 |
| ARSBFGLNGS102860T/C | 0.1076771 | 0.08666172 | 1449 | 1.24250 | 0.2143 |
| ARSBFGLNGS102860C/C | −0.3775612 | 0.20169067 | 1449 | −1.87198 | 0.0614 |
| Correlation: | | | | | |

| | | |
|---|---|---|
| | (Intr) | ARSBFGLNGS102860T |
| ARSBFGLNGS102860T/C | −0.411 | |
| ARSBFGLNGS102860C/C | −0.176 | 0.149 |

Standardized Within-Group Residuals:

| | | | | |
|---|---|---|---|---|
| Min | Q1 | Med | Q3 | Max |
| −1.7423037 | −0.7392987 | −0.1149853 | 0.6326461 | 4.7985812 |

Number of Observations: 1454
Number of Groups: 3

| | | | | |
|---|---|---|---|---|
| | numDF | denDF | F-value | p-value |
| (Intercept) | 1 | 1449 | 1699.5231 | <.0001 |
| ARSBFGLNGS102860 | 2 | 1449 | 2.9349 | 0.0535 |

ARSBFGLNGS102860 × HAPMAP53129RS29022984
Linear mixed-effects model fit by REML
Data: vm

| | | |
|---|---|---|
| AIC | BIC | logLik |
| 5387.703 | 5414.103 | −2688.852 |

Random effects:
Formula: ~1|HAPMAP53129RS29022984

| | | |
|---|---|---|
| | (Intercept) | Residual |
| StdDev: | 0.1728967 | 1.533290 |

Fixed effects: list(fixed)

| | | | | | |
|---|---|---|---|---|---|
| | Value | Std. Error | DF | t-value | p-value |
| (Intercept) | 2.5849061 | 0.12519019 | 1449 | 20.647832 | 0.0000 |
| ARSBFGLNGS102860T/C | 0.1063787 | 0.08641538 | 1449 | 1.231016 | 0.2185 |
| ARSBFGLNGS102860C/C | −0.3957398 | 0.20130267 | 1449 | −1.965894 | 0.0495 |
| Correlation: | | | | | |

| | | |
|---|---|---|
| | (Intr) | ARSBFGLNGS102860T |
| ARSBFGLNGS102860T/C | −0.241 | |
| ARSBFGLNGS102860C/C | −0.097 | 0.149 |

Standardized Within-Group Residuals:

| | | | | |
|---|---|---|---|---|
| Min | Q1 | Med | Q3 | Max |
| −1.78173500 | −0.73822749 | −0.09977032 | 0.63137611 | 4.80956631 |

Number of Observations: 1454
Number of Groups: 3

| | | | | |
|---|---|---|---|---|
| | numDF | denDF | F-value | p-value |
| (Intercept) | 1 | 1449 | 461.6457 | <.0001 |
| ARSBFGLNGS102860 | 2 | 1449 | 3.1199 | 0.0445 |

ARSBFGLNGS102860 × ARSBFGLNGS68110
Linear mixed-effects model fit by REML
Data: vm

| | | |
|---|---|---|
| AIC | BIC | logLik |
| 5357.587 | 5383.987 | −2673.793 |

Random effects:
Formula: ~1|ARSBFGLNGS68110

| | | |
|---|---|---|
| | (Intercept) | Residual |
| StdDev: | 0.3127075 | 1.515663 |

Fixed effects: list(fixed)

| | | | | | |
|---|---|---|---|---|---|
| | Value | Std. Error | DF | t-value | p-value |
| (Intercept) | 2.6186201 | 0.18835951 | 1449 | 13.902245 | 0.0000 |
| ARSBFGLNGS102860T/C | 0.1264738 | 0.08543434 | 1449 | 1.480362 | 0.1390 |
| ARSBFGLNGS102860C/C | −0.3269216 | 0.19930080 | 1449 | −1.640343 | 0.1012 |
| Correlation: | | | | | |

| | | |
|---|---|---|
| | (Intr) | ARSBFGLNGS102860T |
| ARSBFGLNGS102860T/C | −0.160 | |
| ARSBFGLNGS102860C/C | −0.073 | 0.150 |

Standardized Within-Group Residuals:

| | | | | |
|---|---|---|---|---|
| Min | Q1 | Med | Q3 | Max |
| −1.97664122 | −0.72011210 | −0.09998811 | 0.61395631 | 4.70457440 |

Number of Observations: 1454
Number of Groups: 3

| | | | | |
|---|---|---|---|---|
| | numDF | denDF | F-value | p-value |
| (Intercept) | 1 | 1449 | 202.98144 | <.0001 |
| ARSBFGLNGS102860 | 2 | 1449 | 2.87099 | 0.057 |

ARSBFGLNGS102860 × HAPMAP49592BTA38891
Linear mixed-effects model fit by REML
Data: vm

| | | |
|---|---|---|
| AIC | BIC | logLik |
| 5394.221 | 5420.621 | −2692.110 |

Random effects:
Formula: ~1|HAPMAP49592BTA38891

| | | |
|---|---|---|
| | (Intercept) | Residual |
| StdDev: | 0.0001631192 | 1.538043 |

Fixed effects: list(fixed)

| | | | | | |
|---|---|---|---|---|---|
| | Value | Std. Error | DF | t-value | p-value |
| (Intercept) | 2.6613188 | 0.05098589 | 1449 | 52.19716 | 0.0000 |
| ARSBFGLNGS102860T/C | 0.1125401 | 0.08664526 | 1449 | 1.29886 | 0.1942 |
| ARSBFGLNGS102860C/C | −0.3822865 | 0.20187629 | 1449 | −1.89367 | 0.0585 |
| Correlation: | | | | | |

| | | |
|---|---|---|
| | (Intr) | ARSBFGLNGS102860T |
| ARSBFGLNGS102860T/C | −0.588 | |
| ARSBFGLNGS102860C/C | −0.253 | 0.149 |

Standardized Within-Group Residuals:

| | | | | |
|---|---|---|---|---|
| Min | Q1 | Med | Q3 | Max |
| −1.7384811 | −0.7550625 | −0.1048857 | 0.6103088 | 4.8364582 |

Number of Observations: 1454
Number of Groups: 3

| | | | | |
|---|---|---|---|---|
| | numDF | denDF | F-value | p-value |
| (Intercept) | 1 | 1449 | 4422.278 | <.0001 |
| ARSBFGLNGS102860 | 2 | 1449 | 3.070 | 0.0467 |

ARSBFGLNGS102860 × ARSBFGLNGS30157
Linear mixed-effects model fit by REML
Data: vm

| | | |
|---|---|---|
| AIC | BIC | logLik |
| 5389.727 | 5416.127 | −2689.864 |

Random effects:
Formula: ~1|ARSBFGLNGS30157

| | | |
|---|---|---|
| | (Intercept) | Residual |
| StdDev: | 0.2003927 | 1.534364 |

Fixed effects: list(fixed)

| | | | | | |
|---|---|---|---|---|---|
| | Value | Std. Error | DF | t-value | p-value |
| (Intercept) | 2.7788986 | 0.14759193 | 1449 | 18.828256 | 0.0000 |
| ARSBFGLNGS102860T/C | 0.1092501 | 0.08645507 | 1449 | 1.263664 | 0.2066 |
| ARSBFGLNGS102860C/C | −0.4026191 | 0.20158237 | 1449 | −1.997293 | 0.0460 |
| Correlation: | | | | | |

| | | |
|---|---|---|
| | (Intr) | ARSBFGLNGS102860T |
| ARSBFGLNGS102860T/C | −0.201 | |
| ARSBFGLNGS102860C/C | −0.090 | 0.149 |

Standardized Within-Group Residuals:

| | | | | |
|---|---|---|---|---|
| Min | Q1 | Med | Q3 | Max |
| −1.79359277 | −0.74102842 | −0.09305058 | 0.63889153 | 4.88120402 |

Number of Observations: 1454
Number of Groups: 3

| | | | | |
|---|---|---|---|---|
| | numDF | denDF | F-value | p-value |
| (Intercept) | 1 | 1449 | 375.6549 | <.0001 |
| ARSBFGLNGS102860 | 2 | 1449 | 3.2420 | 0.0394 |

ARSBFGLNGS102860 × HAPMAP30097BTC007678
Linear mixed-effects model fit by REML
Data: vm

| | | |
|---|---|---|
| AIC | BIC | logLik |
| 5394.221 | 5420.621 | −2692.110 |

Random effects:
Formula: ~1|HAPMAP30097BTC007678

| | | |
|---|---|---|
| | (Intercept) | Residual |
| StdDev: | 0.0001338598 | 1.538043 |

Fixed effects: list(fixed)

| | | | | | |
|---|---|---|---|---|---|
| | Value | Std. Error | DF | t-value | p-value |
| (Intercept) | 2.6613188 | 0.05098576 | 1449 | 52.19729 | 0.0000 |
| ARSBFGLNGS102860T/C | 0.1125402 | 0.08664508 | 1449 | 1.29886 | 0.1942 |
| ARSBFGLNGS102860C/C | −0.3822864 | 0.20187619 | 1449 | −1.89367 | 0.0585 |

Correlation:

| | | |
|---|---|---|
| | (Intr) | ARSBFGLNGS102860T |
| ARSBFGLNGS102860T/C | −0.588 | |
| ARSBFGLNGS102860C/C | −0.253 | 0.149 |

Standardized Within-Group Residuals:

| | | | | |
|---|---|---|---|---|
| Min | Q1 | Med | Q3 | Max |
| −1.7384811 | −0.7550625 | −0.1048856 | 0.6103089 | 4.8364582 |

Number of Observations: 1454
Number of Groups: 3

| | | | | |
|---|---|---|---|---|
| | numDF | denDF | F-value | p-value |
| (Intercept) | 1 | 1449 | 4422.284 | <.0001 |
| ARSBFGLNGS102860 | 2 | 1449 | 3.070 | 0.0467 |

BFGLNGS119018 × ARSBFGLNGS102860
Linear mixed-effects model fit by REML
Data: vm

| | | |
|---|---|---|
| AIC | BIC | logLik |
| 5395.28 | 5421.68 | −2692.64 |

Random effects:
Formula: ~1|ARSBFGLNGS102860

| | | |
|---|---|---|
| | (Intercept) | Residual |
| StdDev: | 0.1641012 | 1.537593 |

Fixed effects: list(fixed)

| | | | | | |
|---|---|---|---|---|---|
| | Value | Std. Error | DF | t-value | p-value |
| (Intercept) | 2.6786085 | 0.11412993 | 1449 | 23.469816 | 0.0000 |
| BFGLNGS119018G/A | −0.1678354 | 0.09375113 | 1449 | −1.790223 | 0.0736 |
| BFGLNGS119018A/A | −0.1786071 | 0.28949097 | 1449 | −0.616969 | 0.5374 |
| Correlation: | | | | | |

| | | |
|---|---|---|
| | (Intr) | BFGLNGS119018G |
| BFGLNGS119018G/A | −0.213 | |
| BFGLNGS119018A/A | −0.068 | 0.083 |

Standardized Within-Group Residuals:

| | | | | |
|---|---|---|---|---|
| Min | Q1 | Med | Q3 | Max |
| −1.7573702 | −0.7813275 | −0.1309604 | 0.6284384 | 4.8118298 |

Number of Observations: 1454
Number of Groups: 3

| | | | | |
|---|---|---|---|---|
| | numDF | denDF | F-value | p-value |
| (Intercept) | 1 | 1449 | 558.8227 | <.0001 |
| BFGLNGS119018 | 2 | 1449 | 1.7129 | 0.1807 |

BFGLNGS119018 × BFGLNGS119018
Linear mixed-effects model fit by REML
Data: vm

| | | |
|---|---|---|
| AIC | BIC | logLik |
| 5396.247 | 5422.647 | −2693.124 |

Random effects:
Formula: ~1|BFGLNGS119018

| | | |
|---|---|---|
| | (Intercept) | Residual |
| StdDev: | 0.1864887 | 1.539591 |

Fixed effects: list(fixed)

| | | | | | |
|---|---|---|---|---|---|
| | Value | Std. Error | DF | t-value | p-value |
| (Intercept) | 2.7261278 | 0.1923689 | 1451 | 14.171353 | 0 |
| BFGLNGS119018G/A | −0.1629699 | 0.2799105 | 0 | −0.582222 | NaN |
| BFGLNGS119018A/A | −0.1675071 | 0.3918159 | 0 | −0.427515 | NaN |

Correlation:

| | | |
|---|---|---|
| | (Intr) | BFGLNGS119018G |
| BFGLNGS119018G/A | −0.687 | |
| BFGLNGS119018A/A | −0.491 | 0.337 |

Standardized Within-Group Residuals:

| | | | | |
|---|---|---|---|---|
| Min | Q1 | Med | Q3 | Max |
| −1.7057308 | −0.7547612 | −0.1264250 | 0.6325525 | 4.7895007 |

Number of Observations: 1454
Number of Groups: 3

| | | | | |
|---|---|---|---|---|
| | numDF | denDF | F-value | p-value |
| (Intercept) | 1 | 1451 | 415.5197 | <.0001 |
| BFGLNGS119018 | 2 | 0 | 0.1996 | NaN |

BFGLNGS119018 × ARSBFGLBAC20850
Linear mixed-effects model fit by REML
Data: vm

| | | |
|---|---|---|
| AIC | BIC | logLik |
| 5396.247 | 5422.647 | −2693.124 |

Random effects:
Formula: ~1|ARSBFGLBAC20850

| | | |
|---|---|---|
| | (Intercept) | Residual |
| StdDev: | 9.472008e−05 | 1.539591 |

Fixed effects: list(fixed)

| | | | | | |
|---|---|---|---|---|---|
| | Value | Std. Error | DF | t-value | p-value |
| (Intercept) | 2.7261278 | 0.04719926 | 1449 | 57.75785 | 0.0000 |
| BFGLNGS119018G/A | −0.1629699 | 0.09377528 | 1449 | −1.73788 | 0.0824 |
| BFGLNGS119018A/A | −0.1675071 | 0.28976480 | 1449 | −0.57808 | 0.5633 |
| Correlation: | | | | | |

| | | |
|---|---|---|
| | (Intr) | BFGLNGS119018G |
| BFGLNGS119018G/A | −0.503 | |
| BFGLNGS119018A/A | −0.163 | 0.082 |

Standardized Within-Group Residuals:

| | | | | |
|---|---|---|---|---|
| Min | Q1 | Med | Q3 | Max |
| −1.7057308 | −0.7547612 | −0.1264250 | 0.6325525 | 4.7895007 |

Number of Observations: 1454
Number of Groups: 3

| | | | | |
|---|---|---|---|---|
| | numDF | denDF | F-value | p-value |
| (Intercept) | 1 | 1449 | 4413.413 | <.0001 |
| BFGLNGS119018 | 2 | 1449 | 1.606 | 0.2011 |

BFGLNGS119018 × ARSBFGLNGS100843
Linear mixed-effects model fit by REML
Data: vm

| | | |
|---|---|---|
| AIC | BIC | logLik |
| 5394.962 | 5421.362 | −2692.481 |

Random effects:
Formula: ~1|ARSBFGLNGS100843

| | | |
|---|---|---|
| | (Intercept) | Residual |
| StdDev: | 0.1321400 | 1.538157 |

Fixed effects: list(fixed)

| | | | | | |
|---|---|---|---|---|---|
| | Value | Std. Error | DF | t-value | p-value |
| (Intercept) | 2.8028752 | 0.10776413 | 1449 | 26.009352 | 0.0000 |
| BFGLNGS119018G/A | −0.1662953 | 0.09371191 | 1449 | −1.774538 | 0.0762 |
| BFGLNGS119018A/A | −0.1661287 | 0.28952915 | 1449 | −0.573789 | 0.5662 |
| Correlation: | | | | | |

| | | |
|---|---|---|
| | (Intr) | BFGLNGS119018G |
| BFGLNGS119018G/A | −0.227 | |
| BFGLNGS119018A/A | −0.075 | 0.082 |

Standardized Within-Group Residuals:

| | | | | |
|---|---|---|---|---|
| Min | Q1 | Med | Q3 | Max |
| −1.7934706 | −0.7396495 | −0.1325132 | 0.5882265 | 4.8084636 |

Number of Observations: 1454
Number of Groups: 3

| | | | | |
|---|---|---|---|---|
| | numDF | denDF | F-value | p-value |
| (Intercept) | 1 | 1449 | 692.4013 | <.0001 |
| BFGLNGS119018 | 2 | 1449 | 1.6670 | 0.1892 |

BFGLNGS119018 × ARSBFGLNGS97162
Linear mixed-effects model fit by REML
Data: vm

| | | |
|---|---|---|
| AIC | BIC | logLik |
| 5392.297 | 5418.697 | −2691.149 |

Random effects:
Formula: ~1|ARSBFGLNGS97162

| | | |
|---|---|---|
| | (Intercept) | Residual |
| StdDev: | 0.1598071 | 1.536380 |

Fixed effects: list(fixed)

| | | | | | |
|---|---|---|---|---|---|
| | Value | Std. Error | DF | t-value | p-value |
| (Intercept) | 2.7931875 | 0.12086728 | 1449 | 23.109541 | 0.0000 |
| BFGLNGS119018G/A | −0.1473931 | 0.09379437 | 1449 | −1.571450 | 0.1163 |
| BFGLNGS119018A/A | −0.1688119 | 0.28916396 | 1449 | −0.583793 | 0.5595 |
| Correlation: | | | | | |

| | | |
|---|---|---|
| | (Intr) | BFGLNGS119018G |
| BFGLNGS119018G/A | −0.183 | |
| BFGLNGS119018A/A | −0.062 | 0.082 |

Standardized Within-Group Residuals:

| | | | | |
|---|---|---|---|---|
| Min | Q1 | Med | Q3 | Max |
| −1.8009440 | −0.7580530 | −0.1071722 | 0.6087967 | 4.8395221 |

Number of Observations: 1454
Number of Groups: 3

| | | | | |
|---|---|---|---|---|
| | numDF | denDF | F-value | p-value |
| (Intercept) | 1 | 1449 | 539.1168 | <.0001 |
| BFGLNGS119018 | 2 | 1449 | 1.3392 | 0.2624 |

BFGLNGS119018 × Hapmap42294BTA69421
Linear mixed-effects model fit by REML
Data: vm

| | | |
|---|---|---|
| AIC | BIC | logLik |
| 5395.067 | 5421.467 | −2692.533 |

Random effects:
Formula: ~1|Hapmap42294BTA69421

| | | |
|---|---|---|
| | (Intercept) | Residual |
| StdDev: | 0.09249728 | 1.537959 |

Fixed effects: list(fixed)

| | | | | | |
|---|---|---|---|---|---|
| | Value | Std. Error | DF | t-value | p-value |
| (Intercept) | 2.7227829 | 0.07231266 | 1449 | 37.65292 | 0.0000 |
| BFGLNGS119018G/A | −0.1739019 | 0.09388267 | 1449 | −1.85233 | 0.0642 |
| BFGLNGS119018A/A | −0.1670474 | 0.28946430 | 1449 | −0.57709 | 0.5640 |
| Correlation: | | | | | |

| | | |
|---|---|---|
| | (Intr) | BFGLNGS119018G |
| BFGLNGS119018G/A | −0.326 | |
| BFGLNGS119018A/A | −0.105 | 0.082 |

Standardized Within-Group Residuals:

| | | | | |
|---|---|---|---|---|
| Min | Q1 | Med | Q3 | Max |
| −1.7580033 | −0.7580464 | −0.1292418 | 0.6031577 | 4.7441225 |

Number of Observations: 1454
Number of Groups: 3

| | | | | |
|---|---|---|---|---|
| | numDF | denDF | F-value | p-value |
| (Intercept) | 1 | 1449 | 1543.6890 | <.0001 |
| BFGLNGS119018 | 2 | 1449 | 1.8068 | 0.1646 |

BFGLNGS119018 × ARSBFGLBAC2384
Linear mixed-effects model fit by REML
Data: vm

| | | |
|---|---|---|
| AIC | BIC | logLik |
| 5394.964 | 5421.364 | −2692.482 |

Random effects:
Formula: ~1|ARSBFGLBAC2384

| | | |
|---|---|---|
| | (Intercept) | Residual |
| StdDev: | 0.0822645 | 1.538139 |

Fixed effects: list(fixed)

| | | | | | |
|---|---|---|---|---|---|
| | Value | Std. Error | DF | t-value | p-value |
| (Intercept) | 2.7226230 | 0.07001387 | 1449 | 38.88691 | 0.0000 |
| BFGLNGS119018G/A | −0.1542599 | 0.09383569 | 1449 | −1.64394 | 0.1004 |
| BFGLNGS119018A/A | −0.1498359 | 0.28968832 | 1449 | −0.51723 | 0.6051 |
| Correlation: | | | | | |

| | | |
|---|---|---|
| | (Intr) | BFGLNGS119018G |
| BFGLNGS119018G/A | −0.339 | |
| BFGLNGS119018A/A | −0.111 | 0.084 |

Standardized Within-Group Residuals:

| | | | | |
|---|---|---|---|---|
| Min | Q1 | Med | Q3 | Max |
| −1.7464471 | −0.7632703 | −0.1171203 | 0.6020157 | 4.7549150 |

Number of Observations: 1454
Number of Groups: 3

| | | | | |
|---|---|---|---|---|
| | numDF | denDF | F-value | p-value |
| (Intercept) | 1 | 1449 | 1669.6821 | <.0001 |
| BFGLNGS119018 | 2 | 1449 | 1.4237 | 0.2412 |

BFGLNGS119018 × BTB01553536
Linear mixed-effects model fit by REML
Data: vm

| | | |
|---|---|---|
| AIC | BIC | logLik |
| 5394.229 | 5420.629 | −2692.115 |

Random effects:
Formula: ~1|BTB01553536

| | | |
|---|---|---|
| | (Intercept) | Residual |
| StdDev: | 0.0944875 | 1.537465 |

Fixed effects: list(fixed)

| | | | | | |
|---|---|---|---|---|---|
| | Value | Std. Error | DF | t-value | p-value |
| (Intercept) | 2.7287298 | 0.07289937 | 1449 | 37.43146 | 0.0000 |
| BFGLNGS119018G/A | −0.1667822 | 0.09366529 | 1449 | −1.78062 | 0.0752 |
| BFGLNGS119018A/A | −0.1798502 | 0.28945100 | 1449 | −0.62135 | 0.5345 |
| Correlation: | | | | | |

| | | |
|---|---|---|
| | (Intr) | BFGLNGS119018G |
| BFGLNGS119018G/A | −0.325 | |
| BFGLNGS119018A/A | −0.107 | 0.082 |

Standardized Within-Group Residuals:

| | | | | |
|---|---|---|---|---|
| Min | Q1 | Med | Q3 | Max |
| −1.7570242 | −0.7493359 | −0.1079488 | 0.6250846 | 4.7471908 |

Number of Observations: 1454
Number of Groups: 3

| | | | | |
|---|---|---|---|---|
| | numDF | denDF | F-value | p-value |
| (Intercept) | 1 | 1449 | 1526.9201 | <.0001 |
| BFGLNGS119018 | 2 | 1449 | 1.6987 | 0.1833 |

BFGLNGS119018 × HAPMAP53129RS29022984
Linear mixed-effects model fit by REML
Data: vm

| | | |
|---|---|---|
| AIC | BIC | logLik |
| 5391.402 | 5417.802 | −2690.701 |

Random effects:
Formula: ~1|HAPMAP53129RS29022984

| | | |
|---|---|---|
| | (Intercept) | Residual |
| StdDev: | 0.1590200 | 1.535851 |

Fixed effects: list(fixed)

| | | | | | |
|---|---|---|---|---|---|
| | Value | Std. Error | DF | t-value | p-value |
| (Intercept) | 2.6484552 | 0.11825482 | 1449 | 22.396172 | 0.0000 |
| BFGLNGS119018G/A | −0.1172026 | 0.09522464 | 1449 | −1.230802 | 0.2186 |
| BFGLNGS119018A/A | −0.1226030 | 0.29061334 | 1449 | −0.421877 | 0.6732 |
| Correlation: | | | | | |

| | | |
|---|---|---|
| | (Intr) | BFGLNGS119018G |
| BFGLNGS119018G/A | −0.262 | |
| BFGLNGS119018A/A | −0.115 | 0.095 |

Standardized Within-Group Residuals:

| | | | | |
|---|---|---|---|---|
| Min | Q1 | Med | Q3 | Max |
| −1.7374994 | −0.7499251 | −0.1097370 | 0.6064785 | 4.7735502 |

Number of Observations: 1454
Number of Groups: 3

| | | | | |
|---|---|---|---|---|
| | numDF | denDF | F-value | p-value |
| (Intercept) | 1 | 1449 | 526.3699 | <.0001 |
| BFGLNGS119018 | 2 | 1449 | 0.8045 | 0.4475 |

BFGLNGS119018 × ARSBFGLNGS68110
Linear mixed-effects model fit by REML
Data: vm

| | | |
|---|---|---|
| AIC | BIC | logLik |
| 5362.287 | 5388.687 | −2676.144 |

Random effects:
Formula: ~1|ARSBFGLNGS68110

| | | |
|---|---|---|
| | (Intercept) | Residual |
| StdDev: | 0.3166596 | 1.518617 |

Fixed effects: list(fixed)

| | | | | | |
|---|---|---|---|---|---|
| | Value | Std. Error | DF | t-value | p-value |
| (Intercept) | 2.6446177 | 0.19002562 | 1449 | 13.917164 | 0.0000 |
| BFGLNGS119018G/A | −0.0024201 | 0.09597065 | 1449 | −0.025217 | 0.9799 |
| BFGLNGS119018A/A | 0.0576499 | 0.29059406 | 1449 | 0.198386 | 0.8428 |
| Correlation: | | | | | |

| | | |
|---|---|---|
| | (Intr) | BFGLNGS119018G |
| BFGLNGS119018G/A | −0.141 | |
| BFGLNGS119018A/A | −0.061 | 0.115 |

Standardized Within-Group Residuals:

| | | | | |
|---|---|---|---|---|
| Min | Q1 | Med | Q3 | Max |
| −1.9092664 | −0.7239771 | −0.1093065 | 0.6062195 | 4.6756745 |

Number of Observations: 1454
Number of Groups: 3

| | | | | |
|---|---|---|---|---|
| | numDF | denDF | F-value | p-value |
| (Intercept) | 1 | 1449 | 198.16201 | <.0001 |
| BFGLNGS119018 | 2 | 1449 | 0.02085 | 0.9794 |

BFGLNGS119018 × HAPMAP49592BTA38891
Linear mixed-effects model fit by REML
Data: vm

| | | |
|---|---|---|
| AIC | BIC | logLik |
| 5396.126 | 5422.526 | −2693.063 |

Random effects:
Formula: ~1|HAPMAP49592BTA38891

| | | |
|---|---|---|
| | (Intercept) | Residual |
| StdDev: | 0.06520418 | 1.539094 |

Fixed effects: list(fixed)

| | | | | | |
|---|---|---|---|---|---|
| | Value | Std. Error | DF | t-value | p-value |
| (Intercept) | 2.7236083 | 0.06613376 | 1449 | 41.18333 | 0.0000 |
| BFGLNGS119018G/A | −0.1645091 | 0.09378124 | 1449 | −1.75418 | 0.0796 |
| BFGLNGS119018A/A | −0.1695003 | 0.28968738 | 1449 | −0.58511 | 0.5586 |
| Correlation: | | | | | |

| | | |
|---|---|---|
| | (Intr) | BFGLNGS119018G |
| BFGLNGS119018G/A | −0.367 | |
| BFGLNGS119018A/A | −0.119 | 0.082 |

Standardized Within-Group Residuals:

| | | | | |
|---|---|---|---|---|
| Min | Q1 | Med | Q3 | Max |
| −1.7306589 | −0.7560594 | −0.1293860 | 0.6181722 | 4.7995230 |

Number of Observations: 1454
Number of Groups: 3

| | | | | |
|---|---|---|---|---|
| | numDF | denDF | F-value | p-value |
| (Intercept) | 1 | 1449 | 1913.5427 | <.0001 |
| BFGLNGS119018 | 2 | 1449 | 1.6364 | 0.195 |

BFGLNGS119018 × ARSBFGLNGS30157
Linear mixed-effects model fit by REML
Data: vm

| | | |
|---|---|---|
| AIC | BIC | logLik |
| 5392.097 | 5418.497 | −2691.049 |

Random effects:
Formula: ~1|ARSBFGLNGS30157

| | | |
|---|---|---|
| | (Intercept) | Residual |
| StdDev: | 0.1907180 | 1.536146 |

Fixed effects: list(fixed)

| | | | | | |
|---|---|---|---|---|---|
| | Value | Std. Error | DF | t-value | p-value |
| (Intercept) | 2.8342713 | 0.14041024 | 1449 | 20.185645 | 0.0000 |
| BFGLNGS119018G/A | −0.1629756 | 0.09357137 | 1449 | −1.741725 | 0.0818 |
| BFGLNGS119018A/A | −0.1628938 | 0.28912431 | 1449 | −0.563404 | 0.5732 |
| Correlation: | | | | | |

| | | |
|---|---|---|
| | (Intr) | BFGLNGS119018G |
| BFGLNGS119018G/A | −0.164 | |
| BFGLNGS119018A/A | −0.051 | 0.082 |

Standardized Within-Group Residuals:

| | | | | |
|---|---|---|---|---|
| Min | Q1 | Med | Q3 | Max |
| −1.8222600 | −0.7649968 | −0.1140170 | 0.6020608 | 4.8334295 |

Number of Observations: 1454
Number of Groups: 3

| | | | | |
|---|---|---|---|---|
| | numDF | denDF | F-value | p-value |
| (Intercept) | 1 | 1449 | 406.9014 | <.0001 |
| BFGLNGS119018 | 2 | 1449 | 1.6058 | 0.2011 |

BFGLNGS119018 × HAPMAP30097BTC007678
Linear mixed-effects model fit by REML
Data: vm

| | | |
|---|---|---|
| AIC | BIC | logLik |
| 5396.247 | 5422.647 | −2693.124 |

Random effects:
Formula: ~1|HAPMAP30097BTC007678

| | | |
|---|---|---|
| | (Intercept) | Residual |
| StdDev: | 0.0001207614 | 1.539591 |

Fixed effects: list(fixed)

| | | | | | |
|---|---|---|---|---|---|
| | Value | Std. Error | DF | t-value | p-value |
| (Intercept) | 2.7261279 | 0.04719930 | 1449 | 57.75780 | 0.0000 |
| BFGLNGS119018G/A | −0.1629699 | 0.09377528 | 1449 | −1.73788 | 0.0824 |
| BFGLNGS119018A/A | −0.1675071 | 0.28976480 | 1449 | −0.57808 | 0.5633 |
| Correlation: | | | | | |

| | | |
|---|---|---|
| | (Intr) | BFGLNGS119018G |
| BFGLNGS119018G/A | −0.503 | |
| BFGLNGS119018A/A | −0.163 | 0.082 |

Standardized Within-Group Residuals:

| | | | | |
|---|---|---|---|---|
| Min | Q1 | Med | Q3 | Max |
| −1.7057309 | −0.7547612 | −0.1264250 | 0.6325525 | 4.7895007 |

Number of Observations: 1454
Number of Groups: 3

| | | | | |
|---|---|---|---|---|
| | numDF | denDF | F-value | p-value |
| (Intercept) | 1 | 1449 | 4413.403 | <.0001 |
| BFGLNGS119018 | 2 | 1449 | 1.606 | 0.2011 |

ARSBFGLBAC20850 × ARSBFGLNGS102860
Linear mixed-effects model fit by REML
Data: vm

| | | |
|---|---|---|
| AIC | BIC | logLik |
| 5392.987 | 5419.387 | −2691.493 |

Random effects:
Formula: ~1|ARSBFGLNGS102860

| | | |
|---|---|---|
| | (Intercept) | Residual |
| StdDev: | 0.1724558 | 1.537257 |

Fixed effects: list(fixed)

| | | | | | |
|---|---|---|---|---|---|
| | Value | Std. Error | DF | t-value | p-value |
| (Intercept) | 2.6256364 | 0.1171254 | 1449 | 22.417317 | 0.0000 |
| ARSBFGLBAC20850T/C | −0.0264359 | 0.1140649 | 1449 | −0.231762 | 0.8168 |
| ARSBFGLBAC20850C/C | 1.1426915 | 0.5828575 | 1449 | 1.960499 | 0.0501 |
| Correlation: | | | | | |

| | | |
|---|---|---|
| | (Intr) | ARSBFGLBAC20850T |
| ARSBFGLBAC20850T/C | −0.144 | |
| ARSBFGLBAC20850C/C | −0.037 | 0.029 |

Standardized Within-Group Residuals:

| | | | | |
|---|---|---|---|---|
| Min | Q1 | Med | Q3 | Max |
| −1.7253123 | −0.7528562 | −0.1023471 | 0.6165207 | 4.8415226 |

Number of Observations: 1454
Number of Groups: 3

| | | | | |
|---|---|---|---|---|
| | numDF | denDF | F-value | p-value |
| (Intercept) | 1 | 1449 | 515.2169 | <.0001 |
| ARSBFGLBAC20850 | 2 | 1449 | 1.9634 | 0.1408 |

ARSBFGLBAC20850 × BFGLNGS119018
Linear mixed-effects model fit by REML
Data: vm

| | | |
|---|---|---|
| AIC | BIC | logLik |
| 5393.128 | 5419.528 | −2691.564 |

Random effects:
Formula: ~1|BFGLNGS119018

| | | |
|---|---|---|
| | (Intercept) | Residual |
| StdDev: | 0.08259275 | 1.538331 |

Fixed effects: list(fixed)

| | | | | | |
|---|---|---|---|---|---|
| | Value | Std. Error | DF | t-value | p-value |
| (Intercept) | 2.6531269 | 0.0734232 | 1449 | 36.13474 | 0.0000 |
| ARSBFGLBAC20850T/C | −0.0217429 | 0.1141689 | 1449 | −0.19045 | 0.8490 |
| ARSBFGLBAC20850C/C | 1.1082766 | 0.5831399 | 1449 | 1.90053 | 0.0576 |
| Correlation: | | | | | |

| | | |
|---|---|---|
| | (Intr) | ARSBFGLBAC20850T |
| ARSBFGLBAC20850T/C | −0.235 | |
| ARSBFGLBAC20850C/C | −0.040 | 0.028 |

Standardized Within-Group Residuals:

| | | | | |
|---|---|---|---|---|
| Min | Q1 | Med | Q3 | Max |
| −1.6938884 | −0.7708031 | −0.1337564 | 0.6451837 | 4.8066612 |

Number of Observations: 1454
Number of Groups: 3

| | | | | |
|---|---|---|---|---|
| | numDF | denDF | F-value | p-value |
| (Intercept) | 1 | 1449 | 1385.4535 | <.0001 |
| ARSBFGLBAC20850 | 2 | 1449 | 1.8359 | 0.1598 |

ARSBFGLBAC20850 × ARSBFGLBAC20850
Linear mixed-effects model fit by REML
Data: vm

| | | |
|---|---|---|
| AIC | BIC | logLik |
| 5393.902 | 5420.302 | −2691.951 |

Random effects:
Formula: ~1|ARSBFGLBAC20850

| | | |
|---|---|---|
| | (Intercept) | Residual |
| StdDev: | 0.1864429 | 1.539302 |

Fixed effects: list(fixed)

| | | | | | |
|---|---|---|---|---|---|
| | Value | Std. Error | DF | t-value | p-value |
| (Intercept) | 2.680632 | 0.1915231 | 1451 | 13.996390 | 0 |
| ARSBFGLBAC20850T/C | −0.025233 | 0.2873434 | 0 | −0.087815 | NaN |
| ARSBFGLBAC20850C/C | 1.119368 | 0.6402617 | 0 | 1.748297 | NaN |
| Correlation: | | | | | |

| | | |
|---|---|---|
| | (Intr) | ARSBFGLBAC20850T |
| ARSBFGLBAC20850T/C | −0.667 | |
| ARSBFGLBAC20850C/C | −0.299 | 0.199 |

Standardized Within-Group Residuals:

| | | | | |
|---|---|---|---|---|
| Min | Q1 | Med | Q3 | Max |
| −1.6764949 | −0.7669918 | −0.1173467 | 0.6095572 | 4.8199556 |

Number of Observations: 1454
Number of Groups: 3

| | | | | |
|---|---|---|---|---|
| | numDF | denDF | F-value | p-value |
| (Intercept) | 1 | 1451 | 385.0003 | <.0001 |
| ARSBFGLBAC20850 | 2 | 0 | 1.6274 | NaN |

ARSBFGLBAC20850 × ARSBFGLNGS100843
Linear mixed-effects model fit by REML
Data: vm

| | | |
|---|---|---|
| AIC | BIC | logLik |
| 5392.856 | 5419.256 | −2691.428 |

Random effects:
Formula: ~1|ARSBFGLNGS100843

| | | |
|---|---|---|
| | (Intercept) | Residual |
| StdDev: | 0.1198351 | 1.538080 |

Fixed effects: list(fixed)

| | | | | | |
|---|---|---|---|---|---|
| | Value | Std. Error | DF | t-value | p-value |
| (Intercept) | 2.7448953 | 0.0988816 | 1449 | 27.759419 | 0.0000 |
| ARSBFGLBAC20850T/C | −0.0217587 | 0.1141470 | 1449 | −0.190620 | 0.8489 |
| ARSBFGLBAC20850C/C | 1.1040029 | 0.5833088 | 1449 | 1.892656 | 0.0586 |
| Correlation: | | | | | |

| | | |
|---|---|---|
| | (Intr) | ARSBFGLBAC20850T |
| ARSBFGLBAC20850T/C | −0.159 | |
| ARSBFGLBAC20850C/C | −0.051 | 0.028 |

Standardized Within-Group Residuals:

| | | | | |
|---|---|---|---|---|
| Min | Q1 | Med | Q3 | Max |
| −1.7554448 | −0.7537470 | −0.1035860 | 0.6115912 | 4.8376379 |

Number of Observations: 1454
Number of Groups: 3

| | | | | |
|---|---|---|---|---|
| | numDF | denDF | F-value | p-value |
| (Intercept) | 1 | 1449 | 795.5525 | <.0001 |
| ARSBFGLBAC20850 | 2 | 1449 | 1.8209 | 0.1623 |

ARSBFGLBAC20850 × ARSBFGLNGS97162
Linear mixed-effects model fit by REML
Data: vm

| | | |
|---|---|---|
| AIC | BIC | logLik |
| 5389.532 | 5415.932 | −2689.766 |

Random effects:
Formula: ~1|ARSBFGLNGS97162

| | | |
|---|---|---|
| | (Intercept) | Residual |
| StdDev: | 0.1612326 | 1.535859 |

Fixed effects: list(fixed)

| | | | | | |
|---|---|---|---|---|---|
| | Value | Std. Error | DF | t-value | p-value |
| (Intercept) | 2.751024 | 0.1209525 | 1449 | 22.744655 | 0.0000 |
| ARSBFGLBAC20850T/C | −0.034922 | 0.1140213 | 1449 | −0.306276 | 0.7594 |
| ARSBFGLBAC20850C/C | 1.092467 | 0.5827454 | 1449 | 1.874691 | 0.0610 |
| Correlation: | | | | | |

| | | |
|---|---|---|
| | (Intr) | ARSBFGLBAC20850T |
| ARSBFGLBAC20850T/C | −0.144 | |
| ARSBFGLBAC20850C/C | −0.051 | 0.029 |

Standardized Within-Group Residuals:

| | | | | |
|---|---|---|---|---|
| Min | Q1 | Med | Q3 | Max |
| −1.77935610 | −0.73759369 | −0.08649218 | 0.62971948 | 4.86875934 |

Number of Observations: 1454
Number of Groups: 3

| | | | | |
|---|---|---|---|---|
| | numDF | denDF | F-value | p-value |
| (Intercept) | 1 | 1449 | 531.4500 | <.0001 |
| ARSBFGLBAC20850 | 2 | 1449 | 1.8224 | 0.162 |

ARSBFGLBAC20850 × Hapmap42294BTA69421
Linear mixed-effects model fit by REML
Data: vm

| | | |
|---|---|---|
| AIC | BIC | logLik |
| 5393.245 | 5419.645 | −2691.622 |

Random effects:
Formula: ~1|Hapmap42294BTA69421

| | | |
|---|---|---|
| | (Intercept) | Residual |
| StdDev: | 0.07647803 | 1.538172 |

Fixed effects: list(fixed)

| | | | | | |
|---|---|---|---|---|---|
| | Value | Std. Error | DF | t-value | p-value |
| (Intercept) | 2.6758354 | 0.0632763 | 1449 | 42.28810 | 0.0000 |
| ARSBFGLBAC20850T/C | −0.0218617 | 0.1141518 | 1449 | −0.19151 | 0.8481 |
| ARSBFGLBAC20850C/C | 1.1003659 | 0.5832056 | 1449 | 1.88675 | 0.0594 |
| Correlation: | | | | | |

| | | |
|---|---|---|
| | (Intr) | ARSBFGLBAC20850T |
| ARSBFGLBAC20850T/C | −0.266 | |
| ARSBFGLBAC20850C/C | −0.053 | 0.028 |

Standardized Within-Group Residuals:

| | | | | |
|---|---|---|---|---|
| Min | Q1 | Med | Q3 | Max |
| −1.7148574 | −0.7519929 | −0.1018706 | 0.6255827 | 4.7863651 |

Number of Observations: 1454
Number of Groups: 3

| | | | | |
|---|---|---|---|---|
| | numDF | denDF | F-value | p-value |
| (Intercept) | 1 | 1449 | 1931.8590 | <.0001 |
| ARSBFGLBAC20850 | 2 | 1449 | 1.8099 | 0.164 |

ARSBFGLBAC20850 × ARSBFGLBAC2384
Linear mixed-effects model fit by REML
Data: vm

| | | |
|---|---|---|
| AIC | BIC | logLik |
| 5392.212 | 5418.612 | −2691.106 |

Random effects:
Formula: ~1|ARSBFGLBAC2384

| | | |
|---|---|---|
| | (Intercept) | Residual |
| StdDev: | 0.0894907 | 1.537542 |

Fixed effects: list(fixed)

| | | | | | |
|---|---|---|---|---|---|
| | Value | Std. Error | DF | t-value | p-value |
| (Intercept) | 2.6804059 | 0.0710211 | 1449 | 37.74100 | 0.0000 |
| ARSBFGLBAC20850T/C | −0.0294515 | 0.1141189 | 1449 | −0.25808 | 0.7964 |
| ARSBFGLBAC20850C/C | 1.1205335 | 0.5828375 | 1449 | 1.92255 | 0.0547 |
| Correlation: | | | | | |

| | | |
|---|---|---|
| | (Intr) | ARSBFGLBAC20850T |
| ARSBFGLBAC20850T/C | −0.232 | |
| ARSBFGLBAC20850C/C | −0.042 | 0.029 |

Standardized Within-Group Residuals:

| | | | | |
|---|---|---|---|---|
| Min | Q1 | Med | Q3 | Max |
| −1.72488335 | −0.74930004 | −0.09891117 | 0.61651659 | 4.77900536 |

Number of Observations: 1454
Number of Groups: 3

| | | | | |
|---|---|---|---|---|
| | numDF | denDF | F-value | p-value |
| (Intercept) | 1 | 1449 | 1507.7799 | <.0001 |
| ARSBFGLBAC20850 | 2 | 1449 | 1.8974 | 0.1503 |

ARSBFGLBAC20850 × BTB01553536
Linear mixed-effects model fit by REML
Data: vm

| | | |
|---|---|---|
| AIC | BIC | logLik |
| 5392.118 | 5418.518 | −2691.059 |

Random effects:
Formula: ~1|BTB01553536

| | | |
|---|---|---|
| | (Intercept) | Residual |
| StdDev: | 0.09088197 | 1.537353 |

Fixed effects: list(fixed)

| | | | | | |
|---|---|---|---|---|---|
| | Value | Std. Error | DF | t-value | p-value |
| (Intercept) | 2.6842660 | 0.0691345 | 1449 | 38.82674 | 0.0000 |
| ARSBFGLBAC20850T/C | −0.0395581 | 0.1143143 | 1449 | −0.34605 | 0.7294 |
| ARSBFGLBAC20850C/C | 1.0967461 | 0.5828467 | 1449 | 1.88171 | 0.0601 |
| Correlation: | | | | | |

| | | |
|---|---|---|
| | (Intr) | ARSBFGLBAC20850T |
| ARSBFGLBAC20850T/C | −0.243 | |
| ARSBFGLBAC20850C/C | −0.047 | 0.030 |

Standardized Within-Group Residuals:

| | | | | |
|---|---|---|---|---|
| Min | Q1 | Med | Q3 | Max |
| −1.72559315 | −0.74988996 | −0.09942116 | 0.61609452 | 4.77909482 |

Number of Observations: 1454
Number of Groups: 3

| | | | | |
|---|---|---|---|---|
| | numDF | denDF | F-value | p-value |
| (Intercept) | 1 | 1449 | 1604.0608 | <.0001 |
| ARSBFGLBAC20850 | 2 | 1449 | 1.8516 | 0.1574 |

ARSBFGLBAC20850 × HAPMAP53129RS29022984
Linear mixed-effects model fit by REML
Data: vm

| | | |
|---|---|---|
| AIC | BIC | logLik |
| 5387.862 | 5414.262 | −2688.931 |

Random effects:
Formula: ~1|HAPMAP53129RS29022984

| | | |
|---|---|---|
| | (Intercept) | Residual |
| StdDev: | 0.1684172 | 1.534834 |

Fixed effects: list(fixed)

| | | | | | |
|---|---|---|---|---|---|
| | Value | Std. Error | DF | t-value | p-value |
| (Intercept) | 2.6058084 | 0.1198611 | 1449 | 21.740230 | 0.0000 |
| ARSBFGLBAC20850T/C | −0.0226426 | 0.1139174 | 1449 | −0.198763 | 0.8425 |
| ARSBFGLBAC20850C/C | 1.0599973 | 0.5821202 | 1449 | 1.820925 | 0.0688 |
| Correlation: | | | | | |

| | | |
|---|---|---|
| | (Intr) | ARSBFGLBAC20850T |
| ARSBFGLBAC20850T/C | −0.132 | |
| ARSBFGLBAC20850C/C | −0.018 | 0.029 |

Standardized Within-Group Residuals:

| | | | | |
|---|---|---|---|---|
| Min | Q1 | Med | Q3 | Max |
| −1.7200575 | −0.7427531 | −0.1148864 | 0.6254730 | 4.7953051 |

Number of Observations: 1454
Number of Groups: 3

| | | | | |
|---|---|---|---|---|
| | numDF | denDF | F-value | p-value |
| (Intercept) | 1 | 1449 | 481.0622 | <.0001 |
| ARSBFGLBAC20850 | 2 | 1449 | 1.6894 | 0.185 |

ARSBFGLBAC20850 × ARSBFGLNGS68110
Linear mixed-effects model fit by REML
Data: vm

| | | |
|---|---|---|
| AIC | BIC | logLik |
| 5356.49 | 5382.89 | −2673.245 |

Random effects:
Formula: ~1|ARSBFGLNGS68110

| | | |
|---|---|---|
| | (Intercept) | Residual |
| StdDev: | 0.3172087 | 1.516471 |

Fixed effects: list(fixed)

| | | | | | |
|---|---|---|---|---|---|
| | Value | Std. Error | DF | t-value | p-value |
| (Intercept) | 2.6507138 | 0.1889029 | 1449 | 14.032146 | 0.0000 |
| ARSBFGLBAC20850T/C | −0.0742386 | 0.1128391 | 1449 | −0.657915 | 0.5107 |
| ARSBFGLBAC20850C/C | 1.0950060 | 0.5748103 | 1449 | 1.904987 | 0.0570 |
| Correlation: | | | | | |

| | | |
|---|---|---|
| | (Intr) | ARSBFGLBAC20850T |
| ARSBFGLBAC20850T/C | −0.083 | |
| ARSBFGLBAC20850C/C | −0.017 | 0.029 |

Standardized Within-Group Residuals:

| | | | | |
|---|---|---|---|---|
| Min | Q1 | Med | Q3 | Max |
| −1.91676510 | −0.72979876 | −0.09667612 | 0.59905500 | 4.67749238 |

Number of Observations: 1454
Number of Groups: 3

| | | | | |
|---|---|---|---|---|
| | numDF | denDF | F-value | p-value |
| (Intercept) | 1 | 1449 | 197.53949 | <.0001 |
| ARSBFGLBAC20850 | 2 | 1449 | 2.06937 | 0.1266 |

ARSBFGLBAC20850 × HAPMAP49592BTA38891
Linear mixed-effects model fit by REML
Data: vm

| | | |
|---|---|---|
| AIC | BIC | logLik |
| 5393.824 | 5420.224 | −2691.912 |

Random effects:
Formula: ~1|HAPMAP49592BTA38891

| | | |
|---|---|---|
| | (Intercept) | Residual |
| StdDev: | 0.06498917 | 1.538829 |

Fixed effects: list(fixed)

| | | | | | |
|---|---|---|---|---|---|
| | Value | Std. Error | DF | t-value | p-value |
| (Intercept) | 2.6759930 | 0.0634895 | 1449 | 42.14858 | 0.0000 |
| ARSBFGLBAC20850T/C | −0.0257412 | 0.1141788 | 1449 | −0.22545 | 0.8217 |
| ARSBFGLBAC20850C/C | 1.1216435 | 0.5833495 | 1449 | 1.92276 | 0.0547 |
| Correlation: | | | | | |

| | | |
|---|---|---|
| | (Intr) | ARSBFGLBAC20850T |
| ARSBFGLBAC20850T/C | −0.266 | |
| ARSBFGLBAC20850C/C | −0.059 | 0.029 |

Standardized Within-Group Residuals:

| | | | | |
|---|---|---|---|---|
| Min | Q1 | Med | Q3 | Max |
| −1.6992844 | −0.7593666 | −0.1095221 | 0.6053070 | 4.8292967 |

Number of Observations: 1454
Number of Groups: 3

| | | | | |
|---|---|---|---|---|
| | numDF | denDF | F-value | p-value |
| (Intercept) | 1 | 1449 | 1920.572 | <.0001 |
| ARSBFGLBAC20850 | 2 | 1449 | 1.888 | 0.1517 |

ARSBFGLBAC20850 × ARSBFGLNGS30157
Linear mixed-effects model fit by REML
Data: vm

| | | |
|---|---|---|
| AIC | BIC | logLik |
| 5389.622 | 5416.022 | −2689.811 |

Random effects:
Formula: ~1|ARSBFGLNGS30157

| | | |
|---|---|---|
| | (Intercept) | Residual |
| StdDev: | 0.1973648 | 1.53575 |

Fixed effects: list(fixed)

| | | | | | |
|---|---|---|---|---|---|
| | Value | Std. Error | DF | t-value | p-value |
| (Intercept) | 2.7933975 | 0.1433246 | 1449 | 19.490007 | 0.0000 |
| ARSBFGLBAC20850T/C | −0.0208252 | 0.1139667 | 1449 | −0.182731 | 0.8550 |
| ARSBFGLBAC20850C/C | 1.1393428 | 0.5821544 | 1449 | 1.957114 | 0.0505 |
| Correlation: | | | | | |

| | | |
|---|---|---|
| | (Intr) | ARSBFGLBAC20850T |
| ARSBFGLBAC20850T/C | −0.108 | |
| ARSBFGLBAC20850C/C | −0.019 | 0.029 |

Standardized Within-Group Residuals:

| | | | | |
|---|---|---|---|---|
| Min | Q1 | Med | Q3 | Max |
| −1.79436999 | −0.73459177 | −0.08344417 | 0.63281819 | 4.86527760 |

Number of Observations: 1454
Number of Groups: 3

| | | | | |
|---|---|---|---|---|
| | numDF | denDF | F-value | p-value |
| (Intercept) | 1 | 1449 | 384.9412 | <.0001 |
| ARSBFGLBAC20850 | 2 | 1449 | 1.9438 | 0.1435 |

ARSBFGLBAC20850 × HAPMAP30097BTC007678
Linear mixed-effects model fit by REML
Data: vm

| | | |
|---|---|---|
| AIC | BIC | logLik |
| 5393.902 | 5420.302 | −2691.951 |

Random effects:
Formula: ~1|HAPMAP30097BTC007678

| | | |
|---|---|---|
| | (Intercept) | Residual |
| StdDev: | 0.0005315757 | 1.539302 |

Fixed effects: list(fixed)

| | | | | | |
|---|---|---|---|---|---|
| | Value | Std. Error | DF | t-value | p-value |
| (Intercept) | 2.6806337 | 0.0438223 | 1449 | 61.17050 | 0.0000 |
| ARSBFGLBAC20850T/C | −0.0252356 | 0.1142132 | 1449 | −0.22095 | 0.8252 |
| ARSBFGLBAC20850C/C | 1.1193662 | 0.5834498 | 1449 | 1.91853 | 0.0552 |
| Correlation: | | | | | |

| | | |
|---|---|---|
| | (Intr) | ARSBFGLBAC20850T |
| ARSBFGLBAC20850T/C | −0.384 | |
| ARSBFGLBAC20850C/C | −0.075 | 0.029 |

Standardized Within-Group Residuals:

| | | | | |
|---|---|---|---|---|
| Min | Q1 | Med | Q3 | Max |
| −1.6764969 | −0.7669917 | −0.1173466 | 0.6095569 | 4.8199558 |

Number of Observations: 1454
Number of Groups: 3

| | | | | |
|---|---|---|---|---|
| | numDF | denDF | F-value | p-value |
| (Intercept) | 1 | 1449 | 4414.541 | <.0001 |
| ARSBFGLBAC20850 | 2 | 1449 | 1.879 | 0.1532 |

ARSBFGLNGS100843 × ARSBFGLNGS102860
Linear mixed-effects model fit by REML
Data: vm

| | | |
|---|---|---|
| AIC | BIC | logLik |
| 5392.689 | 5419.089 | −2691.344 |

Random effects:
Formula: ~1|ARSBFGLNGS102860

| | | |
|---|---|---|
| | (Intercept) | Residual |
| StdDev: | 0.1758844 | 1.536761 |

Fixed effects: list(fixed)

| | | | | | |
|---|---|---|---|---|---|
| | Value | Std. Error | DF | t-value | p-value |
| (Intercept) | 2.5913444 | 0.1190417 | 1449 | 21.768383 | 0.0000 |
| ARSBFGLNGS100843G/A | 0.1972795 | 0.1159179 | 1449 | 1.701890 | 0.0890 |
| ARSBFGLNGS100843A/A | 0.6214967 | 0.4287838 | 1449 | 1.449441 | 0.1474 |
| Correlation: | | | | | |

| | | |
|---|---|---|
| | (Intr) | ARSBFGLNGS100843G |
| ARSBFGLNGS100843G/A | −0.144 | |
| ARSBFGLNGS100843A/A | −0.052 | 0.039 |

Standardized Within-Group Residuals:

| | | | | |
|---|---|---|---|---|
| Min | Q1 | Med | Q3 | Max |
| −1.77266958 | −0.73328929 | −0.08297366 | 0.63322084 | 4.86289504 |

Number of Observations: 1454
Number of Groups: 3

| | | | | |
|---|---|---|---|---|
| | numDF | denDF | F-value | p-value |
| (Intercept) | 1 | 1449 | 498.7989 | <.0001 |
| ARSBFGLNGS100843 | 2 | 1449 | 2.4062 | 0.0905 |

ARSBFGLNGS100843 × BFGLNGS119018
Linear mixed-effects model fit by REML
Data: vm

| | | |
|---|---|---|
| AIC | BIC | logLik |
| 5392.604 | 5419.004 | −2691.302 |

Random effects:
Formula: ~1|BFGLNGS119018

| | | |
|---|---|---|
| | (Intercept) | Residual |
| StdDev: | 0.0889001 | 1.537687 |

Fixed effects: list(fixed)

| | | | | | |
|---|---|---|---|---|---|
| | Value | Std. Error | DF | t-value | p-value |
| (Intercept) | 2.6180413 | 0.0766807 | 1449 | 34.14214 | 0.0000 |
| ARSBFGLNGS100843G/A | 0.2011061 | 0.1159875 | 1449 | 1.73386 | 0.0832 |
| ARSBFGLNGS100843A/A | 0.5995032 | 0.4287547 | 1449 | 1.39824 | 0.1623 |
| Correlation: | | | | | |

| | | |
|---|---|---|
| | (Intr) | ARSBFGLNGS100843G |
| ARSBFGLNGS100843G/A | −0.218 | |
| ARSBFGLNGS100843A/A | −0.064 | 0.038 |

Standardized Within-Group Residuals:

| | | | | |
|---|---|---|---|---|
| Min | Q1 | Med | Q3 | Max |
| −1.8067154 | −0.7654721 | −0.1151446 | 0.6002156 | 4.8273441 |

Number of Observations: 1454
Number of Groups: 3

| | | | | |
|---|---|---|---|---|
| | numDF | denDF | F-value | p-value |
| (Intercept) | 1 | 1449 | 1260.539 | <.0001 |
| ARSBFGLNGS100843 | 2 | 1449 | 2.391 | 0.0919 |

ARSBFGLNGS100843 × ARSBFGLBAC20850
Linear mixed-effects model fit by REML
Data: vm

| | | |
|---|---|---|
| AIC | BIC | logLik |
| 5393.58 | 5419.98 | −2691.79 |

Random effects:
Formula: ~1|ARSBFGLBAC20850

| | | |
|---|---|---|
| | (Intercept) | Residual |
| StdDev: | 9.474692e−05 | 1.538823 |

Fixed effects: list(fixed)

| | | | | | |
|---|---|---|---|---|---|
| | Value | Std. Error | DF | t-value | p-value |
| (Intercept) | 2.6491100 | 0.0437704 | 1449 | 60.52293 | 0.0000 |
| ARSBFGLNGS100843G/A | 0.1977192 | 0.1160471 | 1449 | 1.70378 | 0.0886 |
| ARSBFGLNGS100843A/A | 0.5970438 | 0.4290312 | 1449 | 1.39161 | 0.1643 |
| Correlation: | | | | | |

| | | |
|---|---|---|
| | (Intr) | ARSBFGLNGS100843G |
| ARSBFGLNGS100843G/A | −0.377 | |
| ARSBFGLNGS100843A/A | −0.102 | 0.038 |

Standardized Within-Group Residuals:

| | | | | |
|---|---|---|---|---|
| Min | Q1 | Med | Q3 | Max |
| −1.78502008 | −0.74674626 | −0.09689878 | 0.61793345 | 4.84194209 |

Number of Observations: 1454
Number of Groups: 3

| | | | | |
|---|---|---|---|---|
| | numDF | denDF | F-value | p-value |
| (Intercept) | 1 | 1449 | 4417.822 | <.0001 |
| ARSBFGLNGS100843 | 2 | 1449 | 2.332 | 0.0975 |

ARSBFGLNGS100843 × ARSBFGLNGS100843
Linear mixed-effects model fit by REML
Data: vm

| | | |
|---|---|---|
| AIC | BIC | logLik |
| 5393.58 | 5419.98 | −2691.79 |

Random effects:
Formula: ~1|ARSBFGLNGS100843

| | | |
|---|---|---|
| | (Intercept) | Residual |
| StdDev: | 0.1863956 | 1.538823 |

Fixed effects: list(fixed)

| | | | | | |
|---|---|---|---|---|---|
| | Value | Std. Error | DF | t-value | p-value |
| (Intercept) | 2.6491100 | 0.1914658 | 1451 | 13.835941 | 0 |
| ARSBFGLNGS100843G/A | 0.1977192 | 0.2880167 | 0 | 0.686485 | NaN |
| ARSBFGLNGS100843A/A | 0.5970438 | 0.5035419 | 0 | 1.185689 | NaN |
| Correlation: | | | | | |

| | | |
|---|---|---|
| | (Intr) | ARSBFGLNGS100843G |
| ARSBFGLNGS100843G/A | −0.665 | |
| ARSBFGLNGS100843A/A | −0.380 | 0.253 |

Standardized Within-Group Residuals:

| | | | | |
|---|---|---|---|---|
| Min | Q1 | Med | Q3 | Max |
| −1.78502008 | −0.74674626 | −0.09689878 | 0.61793345 | 4.84194209 |

Number of Observations: 1454
Number of Groups: 3

| | | | | |
|---|---|---|---|---|
| | numDF | denDF | F-value | p-value |
| (Intercept) | 1 | 1451 | 413.5139 | <.0001 |
| ARSBFGLNGS100843 | 2 | 0 | 0.7828 | NaN |

ARSBFGLNGS100843 × ARSBFGLNGS97162
Linear mixed-effects model fit by REML
Data: vm

| | | |
|---|---|---|
| AIC | BIC | logLik |
| 5388.666 | 5415.066 | −2689.333 |

Random effects:
Formula: ~1|ARSBFGLNGS97162

| | | |
|---|---|---|
| | (Intercept) | Residual |
| StdDev: | 0.1729721 | 1.535017 |

Fixed effects: list(fixed)

| | | | | | |
|---|---|---|---|---|---|
| | Value | Std. Error | DF | t-value | p-value |
| (Intercept) | 2.7264216 | 0.1280586 | 1449 | 21.290420 | 0.0000 |
| ARSBFGLNGS100843G/A | 0.2015925 | 0.1157940 | 1449 | 1.740958 | 0.0819 |
| ARSBFGLNGS100843A/A | 0.6442679 | 0.4283391 | 1449 | 1.504107 | 0.1328 |
| Correlation: | | | | | |

| | | |
|---|---|---|
| | (Intr) | ARSBFGLNGS100843G |
| ARSBFGLNGS100843G/A | −0.136 | |
| ARSBFGLNGS100843A/A | −0.028 | 0.039 |

Standardized Within-Group Residuals:

| | | | | |
|---|---|---|---|---|
| Min | Q1 | Med | Q3 | Max |
| −1.7636006 | −0.7718350 | −0.1203765 | 0.5962278 | 4.8958538 |

Number of Observations: 1454
Number of Groups: 3

| | | | | |
|---|---|---|---|---|
| | numDF | denDF | F-value | p-value |
| (Intercept) | 1 | 1449 | 473.7726 | <.0001 |
| ARSBFGLNGS100843 | 2 | 1449 | 2.5482 | 0.0786 |

ARSBFGLNGS100843 × Hapmap42294BTA69421
Linear mixed-effects model fit by REML
Data: vm

| | | |
|---|---|---|
| AIC | BIC | logLik |
| 5392.609 | 5419.009 | −2691.304 |

Random effects:
Formula: ~1|Hapmap42294BTA69421

| | | |
|---|---|---|
| | (Intercept) | Residual |
| StdDev: | 0.0850974 | 1.537404 |

Fixed effects: list(fixed)

| | | | | | |
|---|---|---|---|---|---|
| | Value | Std. Error | DF | t-value | p-value |
| (Intercept) | 2.6443061 | 0.0668625 | 1449 | 39.54841 | 0.0000 |
| ARSBFGLNGS100843G/A | 0.2001641 | 0.1159585 | 1449 | 1.72617 | 0.0845 |
| ARSBFGLNGS100843A/A | 0.6138407 | 0.4290591 | 1449 | 1.43067 | 0.1527 |
| Correlation: | | | | | |

| | | |
|---|---|---|
| | (Intr) | ARSBFGLNGS100843G |
| ARSBFGLNGS100843G/A | −0.245 | |
| ARSBFGLNGS100843A/A | −0.061 | 0.039 |

Standardized Within-Group Residuals:

| | | | | |
|---|---|---|---|---|
| Min | Q1 | Med | Q3 | Max |
| −1.7685964 | −0.7277743 | −0.0774339 | 0.5874639 | 4.8021179 |

Number of Observations: 1454
Number of Groups: 3

| | | | | |
|---|---|---|---|---|
| | numDF | denDF | F-value | p-value |
| (Intercept) | 1 | 1449 | 1710.6469 | <.0001 |
| ARSBFGLNGS100843 | 2 | 1449 | 2.4201 | 0.0893 |

ARSBFGLNGS100843 × ARSBFGLBAC2384
Linear mixed-effects model fit by REML
Data: vm

| | | |
|---|---|---|
| AIC | BIC | logLik |
| 5391.502 | 5417.902 | −2690.751 |

Random effects:
Formula: ~1|ARSBFGLBAC2384

| | | |
|---|---|---|
| | (Intercept) | Residual |
| StdDev: | 0.09702632 | 1.536764 |

Fixed effects: list(fixed)

| | | | | | |
|---|---|---|---|---|---|
| | Value | Std. Error | DF | t-value | p-value |
| (Intercept) | 2.6444002 | 0.0747722 | 1449 | 35.36610 | 0.0000 |
| ARSBFGLNGS100843G/A | 0.2073055 | 0.1160217 | 1449 | 1.78678 | 0.0742 |
| ARSBFGLNGS100843A/A | 0.6222168 | 0.4286711 | 1449 | 1.45150 | 0.1469 |
| Correlation: | | | | | |

| | | |
|---|---|---|
| | (Intr) | ARSBFGLNGS100843G |
| ARSBFGLNGS100843G/A | −0.228 | |
| ARSBFGLNGS100843A/A | −0.064 | 0.040 |

Standardized Within-Group Residuals:

| | | | | |
|---|---|---|---|---|
| Min | Q1 | Med | Q3 | Max |
| −1.7750550 | −0.7720267 | −0.1213088 | 0.5944809 | 4.7982364 |

Number of Observations: 1454
Number of Groups: 3

| | | | | |
|---|---|---|---|---|
| | numDF | denDF | F-value | p-value |
| (Intercept) | 1 | 1449 | 1360.2316 | <.0001 |
| ARSBFGLNGS100843 | 2 | 1449 | 2.5503 | 0.0784 |

ARSBFGLNGS100843 × BTB01553536
Linear mixed-effects model fit by REML
Data: vm

| | | |
|---|---|---|
| AIC | BIC | logLik |
| 5391.644 | 5418.044 | −2690.822 |

Random effects:
Formula: ~1|BTB01553536

| | | |
|---|---|---|
| | (Intercept) | Residual |
| StdDev: | 0.09325638 | 1.536758 |

Fixed effects: list(fixed)

| | | | | | |
|---|---|---|---|---|---|
| | Value | Std. Error | DF | t-value | p-value |
| (Intercept) | 2.6509982 | 0.0701665 | 1449 | 37.78155 | 0.0000 |
| ARSBFGLNGS100843G/A | 0.1920517 | 0.1159270 | 1449 | 1.65666 | 0.0978 |
| ARSBFGLNGS100843A/A | 0.6357536 | 0.4289091 | 1449 | 1.48226 | 0.1385 |
| Correlation: | | | | | |

| | | |
|---|---|---|
| | (Intr) | ARSBFGLNGS100843G |
| ARSBFGLNGS100843G/A | −0.235 | |
| ARSBFGLNGS100843A/A | −0.062 | 0.037 |

Standardized Within-Group Residuals:

| | | | | |
|---|---|---|---|---|
| Min | Q1 | Med | Q3 | Max |
| −1.7876860 | −0.7517054 | −0.1394200 | 0.6066968 | 4.8008831 |

Number of Observations: 1454
Number of Groups: 3

| | | | | |
|---|---|---|---|---|
| | numDF | denDF | F-value | p-value |
| (Intercept) | 1 | 1449 | 1553.2228 | <.0001 |
| ARSBFGLNGS100843 | 2 | 1449 | 2.3825 | 0.0927 |

ARSBFGLNGS100843 × HAPMAP53129RS29022984
Linear mixed-effects model fit by REML
Data: vm

| | | |
|---|---|---|
| AIC | BIC | logLik |
| 5386.697 | 5413.097 | −2688.349 |

Random effects:
Formula: ~1|HAPMAP53129RS29022984

| | | |
|---|---|---|
| | (Intercept) | Residual |
| StdDev: | 0.1765835 | 1.533847 |

Fixed effects: list(fixed)

| | | | | | |
|---|---|---|---|---|---|
| | Value | Std. Error | DF | t-value | p-value |
| (Intercept) | 2.5715282 | 0.1243439 | 1449 | 20.680767 | 0.0000 |
| ARSBFGLNGS100843G/A | 0.2028445 | 0.1157181 | 1449 | 1.752920 | 0.0798 |
| ARSBFGLNGS100843A/A | 0.6430139 | 0.4279377 | 1449 | 1.502588 | 0.1332 |
| Correlation: | | | | | |

| | | |
|---|---|---|
| | (Intr) | ARSBFGLNGS100843G |
| ARSBFGLNGS100843G/A | −0.125 | |
| ARSBFGLNGS100843A/A | −0.038 | 0.039 |

Standardized Within-Group Residuals:

| | | | | |
|---|---|---|---|---|
| Min | Q1 | Med | Q3 | Max |
| −1.8344912 | −0.7370933 | −0.1375526 | 0.6301587 | 4.8173089 |

Number of Observations: 1454
Number of Groups: 3

| | | | | |
|---|---|---|---|---|
| | numDF | denDF | F-value | p-value |
| (Intercept) | 1 | 1449 | 446.3274 | <.0001 |
| ARSBFGLNGS100843 | 2 | 1449 | 2.5658 | 0.0772 |

ARSBFGLNGS100843 × ARSBFGLNGS68110
Linear mixed-effects model fit by REML
Data: vm

| | | |
|---|---|---|
| AIC | BIC | logLik |
| 5355.52 | 5381.92 | −2672.76 |

Random effects:
Formula: ~1|ARSBFGLNGS68110

| | | |
|---|---|---|
| | (Intercept) | Residual |
| StdDev: | 0.3199793 | 1.515640 |

Fixed effects: list(fixed)

| | | | | | |
|---|---|---|---|---|---|
| | Value | Std. Error | DF | t-value | p-value |
| (Intercept) | 2.6067662 | 0.1906043 | 1449 | 13.676322 | 0.0000 |
| ARSBFGLNGS100843G/A | 0.2244738 | 0.1144764 | 1449 | 1.960874 | 0.0501 |
| ARSBFGLNGS100843A/A | 0.6069938 | 0.4225712 | 1449 | 1.436430 | 0.1511 |
| Correlation: | | | | | |

| | | |
|---|---|---|
| | (Intr) | ARSBFGLNGS100843G |
| ARSBFGLNGS100843G/A | −0.090 | |
| ARSBFGLNGS100843A/A | −0.023 | 0.038 |

Standardized Within-Group Residuals:

| | | | | |
|---|---|---|---|---|
| Min | Q1 | Med | Q3 | Max |
| −1.9730066 | −0.7334882 | −0.0941517 | 0.6163110 | 4.7069917 |

Number of Observations: 1454
Number of Groups: 3

| | | | | |
|---|---|---|---|---|
| | numDF | denDF | F-value | p-value |
| (Intercept) | 1 | 1449 | 194.31314 | <.0001 |
| ARSBFGLNGS100843 | 2 | 1449 | 2.85007 | 0.0582 |

ARSBFGLNGS100843 × HAPMAP49592BTA38891
Linear mixed-effects model fit by REML
Data: vm

| | | |
|---|---|---|
| AIC | BIC | logLik |
| 5393.538 | 5419.938 | −2691.769 |

Random effects:
Formula: ~1|HAPMAP49592BTA38891

| | | |
|---|---|---|
| | (Intercept) | Residual |
| StdDev: | 0.05042462 | 1.53851 |

Fixed effects: list(fixed)

| | | | | | |
|---|---|---|---|---|---|
| | Value | Std. Error | DF | t-value | p-value |
| (Intercept) | 2.6473037 | 0.0568491 | 1449 | 46.56717 | 0.000 |
| ARSBFGLNGS100843G/A | 0.1968631 | 0.1160304 | 1449 | 1.69665 | 0.090 |
| ARSBFGLNGS100843A/A | 0.5974661 | 0.4290430 | 1449 | 1.39256 | 0.164 |
| Correlation: | | | | | |

| | | |
|---|---|---|
| | (Intr) | ARSBFGLNGS100843G |
| ARSBFGLNGS100843G/A | −0.292 | |
| ARSBFGLNGS100843A/A | −0.087 | 0.039 |

Standardized Within-Group Residuals:

| | | | | |
|---|---|---|---|---|
| Min | Q1 | Med | Q3 | Max |
| −1.7791035 | −0.7411753 | −0.1109246 | 0.6237816 | 4.8486482 |

Number of Observations: 1454
Number of Groups: 3

| | | | | |
|---|---|---|---|---|
| | numDF | denDF | F-value | p-value |
| (Intercept) | 1 | 1449 | 2446.6913 | <.0001 |
| ARSBFGLNGS100843 | 2 | 1449 | 2.3212 | 0.0985 |

ARSBFGLNGS100843 × ARSBFGLNGS30157
Linear mixed-effects model fit by REML
Data: vm

| | | |
|---|---|---|
| AIC | BIC | logLik |
| 5389.884 | 5416.284 | −2689.942 |

Random effects:
Formula: ~1|ARSBFGLNGS30157

| | | |
|---|---|---|
| | (Intercept) | Residual |
| StdDev: | 0.1819122 | 1.535674 |

Fixed effects: list(fixed)

| | | | | | |
|---|---|---|---|---|---|
| | Value | Std. Error | DF | t-value | p-value |
| (Intercept) | 2.7536716 | 0.1344497 | 1449 | 20.481053 | 0.0000 |
| ARSBFGLNGS100843G/A | 0.1774704 | 0.1161070 | 1449 | 1.528508 | 0.1266 |
| ARSBFGLNGS100843A/A | 0.6099189 | 0.4281862 | 1449 | 1.424425 | 0.1545 |
| Correlation: | | | | | |

| | | |
|---|---|---|
| | (Intr) | ARSBFGLNGS100843G |
| ARSBFGLNGS100843G/A | −0.144 | |
| ARSBFGLNGS100843A/A | −0.030 | 0.037 |

Standardized Within-Group Residuals:

| | | | | |
|---|---|---|---|---|
| Min | Q1 | Med | Q3 | Max |
| −1.7692204 | −0.7689449 | −0.1177651 | 0.5882388 | 4.8816487 |

Number of Observations: 1454
Number of Groups: 3

| | | | | |
|---|---|---|---|---|
| | numDF | denDF | F-value | p-value |
| (Intercept) | 1 | 1449 | 439.2439 | <.0001 |
| ARSBFGLNGS100843 | 2 | 1449 | 2.1040 | 0.1223 |

ARSBFGLNGS100843 × HAPMAP30097BTC007678
Linear mixed-effects model fit by REML
Data: vm

| | | |
|---|---|---|
| AIC | BIC | logLik |
| 5393.58 | 5419.98 | −2691.79 |

Random effects:
Formula: ~1|HAPMAP30097BTC007678

| | | |
|---|---|---|
| | (Intercept) | Residual |
| StdDev: | 0.0001160010 | 1.538823 |

Fixed effects: list(fixed)

| | | | | | |
|---|---|---|---|---|---|
| | Value | Std. Error | DF | t-value | p-value |
| (Intercept) | 2.6491101 | 0.0437704 | 1449 | 60.52288 | 0.0000 |
| ARSBFGLNGS100843G/A | 0.1977192 | 0.1160471 | 1449 | 1.70378 | 0.0886 |
| ARSBFGLNGS100843A/A | 0.5970438 | 0.4290312 | 1449 | 1.39161 | 0.1643 |
| Correlation: | | | | | |

| | | |
|---|---|---|
| | (Intr) | ARSBFGLNGS100843G |
| ARSBFGLNGS100843G/A | −0.377 | |
| ARSBFGLNGS100843A/A | −0.102 | 0.038 |

Standardized Within-Group Residuals:

| | | | | |
|---|---|---|---|---|
| Min | Q1 | Med | Q3 | Max |
| −1.78502007 | −0.74674625 | −0.09689877 | 0.61793347 | 4.84194210 |

Number of Observations: 1454
Number of Groups: 3

| | | | | |
|---|---|---|---|---|
| | numDF | denDF | F-value | p-value |
| (Intercept) | 1 | 1449 | 4417.814 | <.0001 |
| ARSBFGLNGS100843 | 2 | 1449 | 2.332 | 0.0975 |

ARSBFGLNGS97162 × ARSBFGLNGS102860
Linear mixed-effects model fit by REML
Data: vm

| | | |
|---|---|---|
| AIC | BIC | logLik |
| 5388.379 | 5414.779 | −2689.189 |

Random effects:
Formula: ~1|ARSBFGLNGS102860

| | | |
|---|---|---|
| | (Intercept) | Residual |
| StdDev: | 0.1329727 | 1.534859 |

Fixed effects: list(fixed)

| | | | | | |
|---|---|---|---|---|---|
| | Value | Std. Error | DF | t-value | p-value |
| (Intercept) | 2.8187265 | 0.1156617 | 1449 | 24.370432 | 0.0000 |
| ARSBFGLNGS97162C/T | −0.2413691 | 0.0899109 | 1449 | −2.684536 | 0.0073 |
| ARSBFGLNGS97162T/T | 0.5902225 | 0.5481729 | 1449 | 1.076709 | 0.2818 |
| Correlation: | | | | | |

| | | |
|---|---|---|
| | (Intr) | ARSBFGLNGS97162C |
| ARSBFGLNGS97162C/T | −0.574 | |
| ARSBFGLNGS97162T/T | −0.093 | 0.119 |

Standardized Within-Group Residuals:

| | | | | |
|---|---|---|---|---|
| Min | Q1 | Med | Q3 | Max |
| −2.0911537 | −0.7675200 | −0.1134601 | 0.6351392 | 4.8970079 |

Number of Observations: 1454
Number of Groups: 3

| | | | | |
|---|---|---|---|---|
| | numDF | denDF | F-value | p-value |
| (Intercept) | 1 | 1449 | 780.4553 | <.0001 |
| ARSBFGLNGS97162 | 2 | 1449 | 4.5901 | 0.0103 |

ARSBFGLNGS97162 × BFGLNGS119018
Linear mixed-effects model fit by REML
Data: vm

| | | |
|---|---|---|
| AIC | BIC | logLik |
| 5388.619 | 5415.019 | −2689.309 |

Random effects:
Formula: ~1|BFGLNGS119018

| | | |
|---|---|---|
| | (Intercept) | Residual |
| StdDev: | 0.07010784 | 1.535736 |

Fixed effects: list(fixed)

| | | | | | |
|---|---|---|---|---|---|
| | Value | Std. Error | DF | t-value | p-value |
| (Intercept) | 2.8255659 | 0.0919857 | 1449 | 30.717458 | 0.0000 |
| ARSBFGLNGS97162C/T | −0.2350188 | 0.0899239 | 1449 | −2.613530 | 0.0091 |
| ARSBFGLNGS97162T/T | 0.6051562 | 0.5483082 | 1449 | 1.103679 | 0.2699 |
| Correlation: | | | | | |

| | | |
|---|---|---|
| | (Intr) | ARSBFGLNGS97162C |
| ARSBFGLNGS97162C/T | −0.711 | |
| ARSBFGLNGS97162T/T | −0.112 | 0.117 |

Standardized Within-Group Residuals:

| | | | | |
|---|---|---|---|---|
| Min | Q1 | Med | Q3 | Max |
| −2.0655220 | −0.7521795 | −0.1010259 | 0.6152430 | 4.8628659 |

Number of Observations: 1454
Number of Groups: 3

| | | | | |
|---|---|---|---|---|
| | numDF | denDF | F-value | p-value |
| (Intercept) | 1 | 1449 | 1690.2936 | <.0001 |
| ARSBFGLNGS97162 | 2 | 1449 | 4.4223 | 0.0122 |

ARSBFGLNGS97162 × ARSBFGLBAC20850
Linear mixed-effects model fit by REML
Data: vm

| | | |
|---|---|---|
| AIC | BIC | logLik |
| 5389.069 | 5415.469 | −2689.535 |

Random effects:
Formula: ~1|ARSBFGLBAC20850

| | | |
|---|---|---|
| | (Intercept) | Residual |
| StdDev: | 9.621617e−05 | 1.536418 |

Fixed effects: list(fixed)

| | | | | | |
|---|---|---|---|---|---|
| | Value | Std. Error | DF | t-value | p-value |
| (Intercept) | 2.8506143 | 0.0761575 | 1449 | 37.43052 | 0.0000 |
| ARSBFGLNGS97162C/T | −0.2402196 | 0.0898439 | 1449 | −2.67374 | 0.0076 |
| ARSBFGLNGS97162T/T | 0.6118857 | 0.5485186 | 1449 | 1.11552 | 0.2648 |
| Correlation: | | | | | |

| | | |
|---|---|---|
| | (Intr) | ARSBFGLNGS97162C |
| ARSBFGLNGS97162C/T | −0.848 | |
| ARSBFGLNGS97162T/T | −0.139 | 0.118 |

Standardized Within-Group Residuals:

| | | | | |
|---|---|---|---|---|
| Min | Q1 | Med | Q3 | Max |
| −2.05835856 | −0.74889382 | −0.09802945 | 0.61792135 | 4.87471726 |

Number of Observations: 1454
Number of Groups: 3

| | | | | |
|---|---|---|---|---|
| | numDF | denDF | F-value | p-value |
| (Intercept) | 1 | 1449 | 4431.658 | <.0001 |
| ARSBFGLNGS97162 | 2 | 1449 | 4.612 | 0.0101 |

ARSBFGLNGS97162 × ARSBFGLNGS100843
Linear mixed-effects model fit by REML
Data: vm

| | | |
|---|---|---|
| AIC | BIC | logLik |
| 5387.868 | 5414.268 | −2688.934 |

Random effects:
Formula: ~1|ARSBFGLNGS100843

| | | |
|---|---|---|
| | (Intercept) | Residual |
| StdDev: | 0.1317969 | 1.535034 |

Fixed effects: list(fixed)

| | | | | | |
|---|---|---|---|---|---|
| | Value | Std. Error | DF | t-value | p-value |
| (Intercept) | 2.9296175 | 0.1236267 | 1449 | 23.697296 | 0.0000 |
| ARSBFGLNGS97162C/T | −0.2435360 | 0.0897896 | 1449 | −2.712297 | 0.0068 |
| ARSBFGLNGS97162T/T | 0.5753793 | 0.5485459 | 1449 | 1.048917 | 0.2944 |
| Correlation: | | | | | |

| | | |
|---|---|---|
| | (Intr) | ARSBFGLNGS97162C |
| ARSBFGLNGS97162C/T | −0.533 | |
| ARSBFGLNGS97162T/T | −0.097 | 0.118 |

Standardized Within-Group Residuals:

| | | | | |
|---|---|---|---|---|
| Min | Q1 | Med | Q3 | Max |
| −2.0234225 | −0.7652983 | −0.1218907 | 0.5985203 | 4.8942860 |

Number of Observations: 1454
Number of Groups: 3

| | | | | |
|---|---|---|---|---|
| | numDF | denDF | F-value | p-value |
| (Intercept) | 1 | 1449 | 695.7899 | <.0001 |
| ARSBFGLNGS97162 | 2 | 1449 | 4.6300 | 0.0099 |

ARSBFGLNGS97162 × ARSBFGLNGS97162
Linear mixed-effects model fit by REML
Data: vm

| | | |
|---|---|---|
| AIC | BIC | logLik |
| 5389.069 | 5415.469 | −2689.535 |

Random effects:
Formula: ~1|ARSBFGLNGS97162

| | | |
|---|---|---|
| | (Intercept) | Residual |
| StdDev: | 0.1861044 | 1.536418 |

Fixed effects: list(fixed)

| | | | | | |
|---|---|---|---|---|---|
| | Value | Std. Error | DF | t-value | p-value |
| (Intercept) | 2.8506143 | 0.2010841 | 1451 | 14.176231 | 0 |
| ARSBFGLNGS97162C/T | −0.2402196 | 0.2781036 | 0 | −0.863777 | NaN |
| ARSBFGLNGS97162T/T | 0.6118857 | 0.6083933 | 0 | 1.005741 | NaN |
| Correlation: | | | | | |

| | | |
|---|---|---|
| | (Intr) | ARSBFGLNGS97162C |
| ARSBFGLNGS97162C/T | −0.723 | |
| ARSBFGLNGS97162T/T | −0.331 | 0.239 |

Standardized Within-Group Residuals:

| | | | | |
|---|---|---|---|---|
| Min | Q1 | Med | Q3 | Max |
| −2.05835856 | −0.74889382 | −0.09802945 | 0.61792135 | 4.87471727 |

Number of Observations: 1454
Number of Groups: 3

| | | | | |
|---|---|---|---|---|
| | numDF | denDF | F-value | p-value |
| (Intercept) | 1 | 1451 | 419.6547 | <.0001 |
| ARSBFGLNGS97162 | 2 | 0 | 1.1522 | NaN |

ARSBFGLNGS97162 × Hapmap42294BTA69421
Linear mixed-effects model fit by REML
Data: vm

| | | |
|---|---|---|
| AIC | BIC | logLik |
| 5388.376 | 5414.776 | −2689.188 |

Random effects:
Formula: ~1|Hapmap42294BTA69421

| | | |
|---|---|---|
| | (Intercept) | Residual |
| StdDev: | 0.07900897 | 1.535234 |

Fixed effects: list(fixed)

| | | | | | |
|---|---|---|---|---|---|
| | Value | Std. Error | DF | t-value | p-value |
| (Intercept) | 2.8454634 | 0.0892817 | 1449 | 31.87063 | 0.0000 |
| ARSBFGLNGS97162C/T | −0.2405695 | 0.0898153 | 1449 | −2.67849 | 0.0075 |
| ARSBFGLNGS97162T/T | 0.5870617 | 0.5483880 | 1449 | 1.07052 | 0.2846 |
| Correlation: | | | | | |

| | | |
|---|---|---|
| | (Intr) | ARSBFGLNGS97162C |
| ARSBFGLNGS97162C/T | −0.719 | |
| ARSBFGLNGS97162T/T | −0.118 | 0.117 |

Standardized Within-Group Residuals:

| | | | | |
|---|---|---|---|---|
| Min | Q1 | Med | Q3 | Max |
| −2.0821605 | −0.7614291 | −0.1100624 | 0.6140254 | 4.8403244 |

Number of Observations: 1454
Number of Groups: 3

| | | | | |
|---|---|---|---|---|
| | numDF | denDF | F-value | p-value |
| (Intercept) | 1 | 1449 | 1866.4963 | <.0001 |
| ARSBFGLNGS97162 | 2 | 1449 | 4.5592 | 0.0106 |

ARSBFGLNGS97162 × ARSBFGLBAC2384
Linear mixed-effects model fit by REML
Data: vm

| | | |
|---|---|---|
| AIC | BIC | logLik |
| 5388.608 | 5415.008 | −2689.304 |

Random effects:
Formula: ~1|ARSBFGLBAC2384

| | | |
|---|---|---|
| | (Intercept) | Residual |
| StdDev: | 0.06073586 | 1.535685 |

Fixed effects: list(fixed)

| | | | | | |
|---|---|---|---|---|---|
| | Value | Std. Error | DF | t-value | p-value |
| (Intercept) | 2.8394502 | 0.0859794 | 1449 | 33.02479 | 0.0000 |
| ARSBFGLNGS97162C/T | −0.2245351 | 0.0907835 | 1449 | −2.47330 | 0.0135 |
| ARSBFGLNGS97162T/T | 0.6251412 | 0.5483781 | 1449 | 1.13998 | 0.2545 |
| Correlation: | | | | | |

| | | |
|---|---|---|
| | (Intr) | ARSBFGLNGS97162C |
| ARSBFGLNGS97162C/T | −0.759 | |
| ARSBFGLNGS97162T/T | −0.126 | 0.119 |

Standardized Within-Group Residuals:

| | | | | |
|---|---|---|---|---|
| Min | Q1 | Med | Q3 | Max |
| −2.0400077 | −0.7516023 | −0.1164035 | 0.6158661 | 4.8485061 |

Number of Observations: 1454
Number of Groups: 3

| | | | | |
|---|---|---|---|---|
| | numDF | denDF | F-value | p-value |
| (Intercept) | 1 | 1449 | 2303.3665 | <.0001 |
| ARSBFGLNGS97162 | 2 | 1449 | 4.1036 | 0.0167 |

ARSBFGLNGS97162 × BTB01553536
Linear mixed-effects model fit by REML
Data: vm

| | | |
|---|---|---|
| AIC | BIC | logLik |
| 5387.876 | 5414.276 | −2688.938 |

Random effects:
Formula: ~1|BTB01553536

| | | |
|---|---|---|
| | (Intercept) | Residual |
| StdDev: | 0.07947534 | 1.534946 |

Fixed effects: list(fixed)

| | | | | | |
|---|---|---|---|---|---|
| | Value | Std. Error | DF | t-value | p-value |
| (Intercept) | 2.8457060 | 0.0893558 | 1449 | 31.84691 | 0.0000 |
| ARSBFGLNGS97162C/T | −0.2319638 | 0.0899008 | 1449 | −2.58022 | 0.0100 |
| ARSBFGLNGS97162T/T | 0.5955161 | 0.5481138 | 1449 | 1.08648 | 0.2774 |
| Correlation: | | | | | |

| | | |
|---|---|---|
| | (Intr) | ARSBFGLNGS97162C |
| ARSBFGLNGS97162C/T | −0.721 | |
| ARSBFGLNGS97162T/T | −0.116 | 0.117 |

Standardized Within-Group Residuals:

| | | | | |
|---|---|---|---|---|
| Min | Q1 | Med | Q3 | Max |
| −2.0076107 | −0.7518792 | −0.1115414 | 0.6162471 | 4.8397730 |

Number of Observations: 1454
Number of Groups: 3

| | | | | |
|---|---|---|---|---|
| | numDF | denDF | F-value | p-value |
| (Intercept) | 1 | 1449 | 1883.5733 | <.0001 |
| ARSBFGLNGS97162 | 2 | 1449 | 4.3045 | 0.0137 |

ARSBFGLNGS97162 × HAPMAP53129RS29022984
Linear mixed-effects model fit by REML
Data: vm

| | | |
|---|---|---|
| AIC | BIC | logLik |
| 5383.277 | 5409.677 | −2686.639 |

Random effects:
Formula: ~1|HAPMAP53129RS29022984

| | | |
|---|---|---|
| | (Intercept) | Residual |
| StdDev: | 0.1656495 | 1.532109 |

Fixed effects: list(fixed)

| | | | | | |
|---|---|---|---|---|---|
| | Value | Std. Error | DF | t-value | p-value |
| (Intercept) | 2.7728786 | 0.1339607 | 1449 | 20.699187 | 0.0000 |
| ARSBFGLNGS97162C/T | −0.2340170 | 0.0896191 | 1449 | −2.611241 | 0.0091 |
| ARSBFGLNGS97162T/T | 0.5577603 | 0.5473134 | 1449 | 1.019088 | 0.3083 |
| Correlation: | | | | | |

| | | |
|---|---|---|
| | (Intr) | ARSBFGLNGS97162C |
| ARSBFGLNGS97162C/T | −0.484 | |
| ARSBFGLNGS97162T/T | −0.070 | 0.117 |

Standardized Within-Group Residuals:

| | | | | |
|---|---|---|---|---|
| Min | Q1 | Med | Q3 | Max |
| −2.0641485 | −0.7363249 | −0.1114299 | 0.6065348 | 4.8490534 |

Number of Observations: 1454
Number of Groups: 3

| | | | | |
|---|---|---|---|---|
| | numDF | denDF | F-value | p-value |
| (Intercept) | 1 | 1449 | 494.1862 | <.0001 |
| ARSBFGLNGS97162 | 2 | 1449 | 4.2979 | 0.0138 |

ARSBFGLNGS97162 × ARSBFGLNGS68110
Linear mixed-effects model fit by REML
Data: vm

| | | |
|---|---|---|
| AIC | BIC | logLik |
| 5355.334 | 5381.734 | −2672.667 |

Random effects:
Formula: ~1|ARSBFGLNGS68110

| | | |
|---|---|---|
| | (Intercept) | Residual |
| StdDev: | 0.3038537 | 1.515638 |

Fixed effects: list(fixed)

| | | | | | |
|---|---|---|---|---|---|
| | Value | Std. Error | DF | t-value | p-value |
| (Intercept) | 2.7661491 | 0.1920986 | 1449 | 14.399632 | 0.0000 |
| ARSBFGLNGS97162C/T | −0.1717625 | 0.0892914 | 1449 | −1.923617 | 0.0546 |
| ARSBFGLNGS97162T/T | 0.6738184 | 0.5412729 | 1449 | 1.244877 | 0.2134 |
| Correlation: | | | | | |

| | | |
|---|---|---|
| | (Intr) | ARSBFGLNGS97162C |
| ARSBFGLNGS97162C/T | −0.338 | |
| ARSBFGLNGS97162T/T | −0.057 | 0.119 |

Standardized Within-Group Residuals:

| | | | | |
|---|---|---|---|---|
| Min | Q1 | Med | Q3 | Max |
| −1.95278062 | −0.73508784 | −0.08868629 | 0.58973281 | 4.72763631 |

Number of Observations: 1454
Number of Groups: 3

| | | | | |
|---|---|---|---|---|
| | numDF | denDF | F-value | p-value |
| (Intercept) | 1 | 1449 | 214.32747 | <.0001 |
| ARSBFGLNGS97162 | 2 | 1449 | 2.95228 | 0.0525 |

ARSBFGLNGS97162 × HAPMAP49592BTA38891
Linear mixed-effects model fit by REML
Data: vm

| | | |
|---|---|---|
| AIC | BIC | logLik |
| 5388.862 | 5415.262 | −2689.431 |

Random effects:
Formula: ~1|HAPMAP49592BTA38891

| | | |
|---|---|---|
| | (Inte rcept) | Residual |
| StdDev: | 0.07375945 | 1.535791 |

Fixed effects: list(fixed)

| | | | | | |
|---|---|---|---|---|---|
| | Value | Std. Error | DF | t-value | p-value |
| (Intercept) | 2.8498367 | 0.0928185 | 1449 | 30.703311 | 0.0000 |
| ARSBFGLNGS97162C/T | −0.2435734 | 0.0899381 | 1449 | −2.708235 | 0.0068 |
| ARSBFGLNGS97162T/T | 0.6027577 | 0.5483724 | 1449 | 1.099176 | 0.2719 |
| Correlation: | | | | | |

| | | |
|---|---|---|
| | (Intr) | ARSBFGLNGS97162C |
| ARSBFGLNGS97162C/T | −0.708 | |
| ARSBFGLNGS97162T/T | −0.116 | 0.118 |

Standardized Within-Group Residuals:

| | | | | |
|---|---|---|---|---|
| Min | Q1 | Med | Q3 | Max |
| −2.0441096 | −0.7519209 | −0.1007907 | 0.6243531 | 4.8880385 |

Number of Observations: 1454
Number of Groups: 3

| | | | | |
|---|---|---|---|---|
| | numDF | denDF | F-value | p-value |
| (Intercept) | 1 | 1449 | 1671.1889 | <.0001 |
| ARSBFGLNGS97162 | 2 | 1449 | 4.6892 | 0.0093 |

ARSBFGLNGS97162 × ARSBFGLNGS30157
Linear mixed-effects model fit by REML
Data: vm

| | | |
|---|---|---|
| AIC | BIC | logLik |
| 5383.46 | 5409.86 | −2686.73 |

Random effects:
Formula: ~1|ARSBFGLNGS30157

| | | |
|---|---|---|
| | (Intercept) | Residual |
| StdDev: | 0.2038271 | 1.532138 |

Fixed effects: list(fixed)

| | | | | | |
|---|---|---|---|---|---|
| | Value | Std. Error | DF | t-value | p-value |
| (Intercept) | 2.9818558 | 0.1598540 | 1449 | 18.653621 | 0.0000 |
| ARSBFGLNGS97162C/T | −0.2622008 | 0.0900891 | 1449 | −2.910462 | 0.0037 |
| ARSBFGLNGS97162T/T | 0.6221702 | 0.5470109 | 1449 | 1.137400 | 0.2556 |
| Correlation: | | | | | |

| | | |
|---|---|---|
| | (Intr) | ARSBFGLNGS97162C |
| ARSBFGLNGS97162C/T | −0.402 | |
| ARSBFGLNGS97162T/T | −0.062 | 0.117 |

Standardized Within-Group Residuals:

| | | | | |
|---|---|---|---|---|
| Min | Q1 | Med | Q3 | Max |
| −2.04326291 | −0.74809790 | −0.09541499 | 0.62253621 | 4.93024341 |

Number of Observations: 1454
Number of Groups: 3

| | | | | |
|---|---|---|---|---|
| | numDF | denDF | F-value | p-value |
| (Intercept) | 1 | 1449 | 365.5991 | <.0001 |
| ARSBFGLNGS97162 | 2 | 1449 | 5.3418 | 0.0049 |

ARSBFGLNGS97162 × HAPMAP30097BTC007678
Linear mixed-effects model fit by REML
Data: vm

| | | |
|---|---|---|
| AIC | BIC | logLik |
| 5389.069 | 5415.469 | −2689.535 |

Random effects:
Formula: ~1|HAPMAP30097BTC007678

| | | |
|---|---|---|
| | (Intercept) | Residual |
| StdDev: | 0.0001228237 | 1.536418 |

Fixed effects: list(fixed)

| | | | | | |
|---|---|---|---|---|---|
| | Value | Std. Error | DF | t-value | p-value |
| (Intercept) | 2.8506143 | 0.0761575 | 1449 | 37.43051 | 0.0000 |
| ARSBFGLNGS97162C/T | −0.2402196 | 0.0898439 | 1449 | −2.67374 | 0.0076 |
| ARSBFGLNGS97162T/T | 0.6118857 | 0.5485186 | 1449 | 1.11552 | 0.2648 |
| Correlation: | | | | | |

| | | |
|---|---|---|
| | (Intr) | ARSBFGLNGS97162C |
| ARSBFGLNGS97162C/T | −0.848 | |
| ARSBFGLNGS97162T/T | −0.139 | 0.118 |

Standardized Within-Group Residuals:

| | | | | |
|---|---|---|---|---|
| Min | Q1 | Med | Q3 | Max |
| −2.05835852 | −0.74889380 | −0.09802943 | 0.61792137 | 4.87471729 |

Number of Observations: 1454
Number of Groups: 3

| | | | | |
|---|---|---|---|---|
| | numDF | denDF | F-value | p-value |
| (Intercept) | 1 | 1449 | 4431.647 | <.0001 |
| ARSBFGLNGS97162 | 2 | 1449 | 4.612 | 0.0101 |

Hapmap42294BTA69421 × ARSBFGLNGS102860
Linear mixed-effects model fit by REML
Data: vm

| | | |
|---|---|---|
| AIC | BIC | logLik |
| 5396.673 | 5423.073 | −2693.336 |

Random effects:
Formula: ~1|ARSBFGLNGS102860

| | | |
|---|---|---|
| | (Intercept) | Residual |
| StdDev: | 0.1259620 | 1.537583 |

Fixed effects: list(fixed)

| | | | | | |
|---|---|---|---|---|---|
| | Value | Std. Error | DF | t-value | p-value |
| (Intercept) | 2.7581713 | 0.10716665 | 1449 | 25.737217 | 0.0000 |
| Hapmap42294BTA69421G/A | −0.1452449 | 0.09027261 | 1449 | −1.608958 | 0.1078 |
| Hapmap42294BTA69421A/A | −0.2169563 | 0.11781464 | 1449 | −1.841505 | 0.0658 |
| Correlation: | | | | | |

| | | |
|---|---|---|
| | (Intr) | H42294BTA69421G |
| Hapmap42294BTA69421G/A | −0.493 | |
| Hapmap42294BTA69421A/A | −0.391 | 0.447 |

Standardized Within-Group Residuals:

| | | | | |
|---|---|---|---|---|
| Min | Q1 | Med | Q3 | Max |
| −1.7356824 | −0.7486787 | −0.0970510 | 0.6183575 | 4.7680308 |

Number of Observations: 1454
Number of Groups: 3

| | | | | |
|---|---|---|---|---|
| | numDF | denDF | F-value | p-value |
| (Intercept) | 1 | 1449 | 846.9056 | <.0001 |
| Hapmap42294BTA69421 | 2 | 1449 | 2.0808 | 0.1252 |

Hapmap42294BTA69421 × BFGLNGS119018
Linear mixed-effects model fit by REML
Data: vm

| | | |
|---|---|---|
| AIC | BIC | logLik |
| 5395.759 | 5422.159 | −2692.880 |

Random effects:
Formula: ~1|BFGLNGS119018

| | | |
|---|---|---|
| | (Intercept) | Residual |
| StdDev: | 0.0979611 | 1.537525 |

Fixed effects: list(fixed)

| | | | | | |
|---|---|---|---|---|---|
| | Value | Std. Error | DF | t-value | p-value |
| (Intercept) | 2.7654421 | 0.09677538 | 1449 | 28.575883 | 0.0000 |
| Hapmap42294BTA69421G/A | −0.1565744 | 0.09037983 | 1449 | −1.732404 | 0.0834 |
| Hapmap42294BTA69421A/A | −0.2387763 | 0.11787924 | 1449 | −2.025601 | 0.0430 |
| Correlation: | | | | | |

| | | |
|---|---|---|
| | (Intr) | H42294BTA69421G |
| Hapmap42294BTA69421G/A | −0.536 | |
| Hapmap42294BTA69421A/A | −0.407 | 0.449 |

Standardized Within-Group Residuals:

| | | | | |
|---|---|---|---|---|
| Min | Q1 | Med | Q3 | Max |
| −1.7773016 | −0.7649120 | −0.1145162 | 0.6046715 | 4.7266572 |

Number of Observations: 1454
Number of Groups: 3

| | | | | |
|---|---|---|---|---|
| | numDF | denDF | F-value | p-value |
| (Intercept) | 1 | 1449 | 1106.5730 | <.0001 |
| Hapmap42294BTA69421 | 2 | 1449 | 2.4755 | 0.0845 |

Hapmap42294BTA69421 × ARSBFGLBAC20850
Linear mixed-effects model fit by REML
Data: vm

| | | |
|---|---|---|
| AIC | BIC | logLik |
| 5397.078 | 5423.478 | −2693.539 |

Random effects:
Formula: ~1|ARSBFGLBAC20850

| | | |
|---|---|---|
| | (Intercept) | Residual |
| StdDev: | 9.45407e−05 | 1.538923 |

Fixed effects: list(fixed)

| | | | | | |
|---|---|---|---|---|---|
| | Value | Std. Error | DF | t-value | p-value |
| (Intercept) | 2.7939759 | 0.06896083 | 1449 | 40.51540 | 0.0000 |
| Hapmap42294BTA69421G/A | −0.1485736 | 0.09032328 | 1449 | −1.64491 | 0.1002 |
| Hapmap42294BTA69421A/A | −0.2266682 | 0.11774707 | 1449 | −1.92504 | 0.0544 |
| Correlation: | | | | | |

| | | |
|---|---|---|
| | (Intr) | H42294BTA69421G |
| Hapmap42294BTA69421G/A | −0.763 | |
| Hapmap42294BTA69421A/A | −0.586 | 0.447 |

Standardized Within-Group Residuals:

| | | | | |
|---|---|---|---|---|
| Min | Q1 | Med | Q3 | Max |
| −1.75055945 | −0.74428833 | −0.09448316 | 0.62030251 | 4.74749217 |

Number of Observations: 1454
Number of Groups: 3

| | | | | |
|---|---|---|---|---|
| | numDF | denDF | F-value | p-value |
| (Intercept) | 1 | 1449 | 4417.246 | <.0001 |
| Hapmap42294BTA69421 | 2 | 1449 | 2.237 | 0.1071 |

Hapmap42294BTA69421 × ARSBFGLNGS100843
Linear mixed-effects model fit by REML
Data: vm

| | | |
|---|---|---|
| AIC | BIC | logLik |
| 5395.8 | 5422.2 | −2692.9 |

Random effects:
Formula: ~1|ARSBFGLNGS100843

| | | |
|---|---|---|
| | (Intercept) | Residual |
| StdDev: | 0.1326903 | 1.537489 |

Fixed effects: list(fixed)

| | | | | | |
|---|---|---|---|---|---|
| | Value | Std. Error | DF | t-value | p-value |
| (Intercept) | 2.8726288 | 0.11963112 | 1449 | 24.012387 | 0.0000 |
| Hapmap42294BTA69421G/A | −0.1524597 | 0.09027468 | 1449 | −1.688842 | 0.0915 |
| Hapmap42294BTA69421A/A | −0.2275604 | 0.11763880 | 1449 | −1.934399 | 0.0533 |
| Correlation: | | | | | |

| | | |
|---|---|---|
| | (Intr) | H42294BTA69421G |
| Hapmap42294BTA69421G/A | −0.453 | |
| Hapmap42294BTA69421A/A | −0.339 | 0.447 |

Standardized Within-Group Residuals:

| | | | | |
|---|---|---|---|---|
| Min | Q1 | Med | Q3 | Max |
| −1.73985845 | −0.76287423 | −0.09453764 | 0.60298886 | 4.76561924 |

Number of Observations: 1454
Number of Groups: 3

| | | | | |
|---|---|---|---|---|
| | numDF | denDF | F-value | p-value |
| (Intercept) | 1 | 1449 | 688.4349 | <.0001 |
| Hapmap42294BTA69421 | 2 | 1449 | 2.2953 | 0.1011 |

Hapmap42294BTA69421 × ARSBFGLNGS97162
Linear mixed-effects model fit by REML
Data: vm

| | | |
|---|---|---|
| AIC | BIC | logLik |
| 5392.537 | 5418.938 | −2691.269 |

Random effects:
Formula: ~1|ARSBFGLNGS97162

| | | |
|---|---|---|
| | (Intercept) | Residual |
| StdDev: | 0.1673823 | 1.535350 |

Fixed effects: list(fixed)

| | | | | | |
|---|---|---|---|---|---|
| | Value | Std. Error | DF | t-value | p-value |
| (Intercept) | 2.8672383 | 0.13494958 | 1449 | 21.246737 | 0.0000 |
| Hapmap42294BTA69421G/A | −0.1431609 | 0.09014325 | 1449 | −1.588149 | 0.1125 |
| Hapmap42294BTA69421A/A | −0.2323932 | 0.11750548 | 1449 | −1.977723 | 0.0481 |
| Correlation: | | | | | |

| | | |
|---|---|---|
| | (Intr) | H42294BTA69421G |
| Hapmap42294BTA69421G/A | −0.379 | |
| Hapmap42294BTA69421A/A | −0.297 | 0.447 |

Standardized Within-Group Residuals:

| | | | | |
|---|---|---|---|---|
| Min | Q1 | Med | Q3 | Max |
| −1.7603385 | −0.7463421 | −0.1225166 | 0.6214245 | 4.7993846 |

Number of Observations: 1454
Number of Groups: 3

| | | | | |
|---|---|---|---|---|
| | numDF | denDF | F-value | p-value |
| (Intercept) | 1 | 1449 | 499.9917 | <.0001 |
| Hapmap42294BTA69421 | 2 | 1449 | 2.2660 | 0.1041 |

Hapmap42294BTA69421 × Hapmap42294BTA69421
Linear mixed-effects model fit by REML
Data: vm

| | | |
|---|---|---|
| AIC | BIC | logLik |
| 5397.078 | 5423.478 | −2693.539 |

Random effects:
Formula: ~1|Hapmap42294BTA69421

| | | |
|---|---|---|
| | (Intercept) | Residual |
| StdDev: | 0.1864078 | 1.538923 |

Fixed effects: list(fixed)

| | | | | | |
|---|---|---|---|---|---|
| | Value | Std. Error | DF | t-value | p-value |
| (Intercept) | 2.7939759 | 0.1987547 | 1451 | 14.057406 | 0 |
| Hapmap42294BTA69421G/A | −0.1485736 | 0.2786647 | 0 | −0.533163 | NaN |
| Hapmap42294BTA69421A/A | −0.2266682 | 0.2887215 | 0 | −0.785076 | NaN |
| Correlation: | | | | | |

| | | |
|---|---|---|
| | (Intr) | H42294BTA69421G |
| Hapmap42294BTA69421G/A | −0.713 | |
| Hapmap42294BTA69421A/A | −0.688 | 0.491 |

Standardized Within-Group Residuals:

| | | | | |
|---|---|---|---|---|
| Min | Q1 | Med | Q3 | Max |
| −1.75055945 | −0.74428833 | −0.09448316 | 0.62030251 | 4.74749217 |

Number of Observations: 1454
Number of Groups: 3

| | | | | |
|---|---|---|---|---|
| | numDF | denDF | F-value | p-value |
| (Intercept) | 1 | 1451 | 530.6874 | <.0001 |
| Hapmap42294BTA69421 | 2 | 0 | 0.3225 | NaN |

Hapmap42294BTA69421 × ARSBFGLBAC2384
Linear mixed-effects model fit by REML
Data: vm

| | | |
|---|---|---|
| AIC | BIC | logLik |
| 5395.194 | 5421.594 | −2692.597 |

Random effects:
Formula: ~1|ARSBFGLBAC2384

| | | |
|---|---|---|
| | (Intercept) | Residual |
| StdDev: | 0.0924061 | 1.537025 |

Fixed effects: list(fixed)

| | | | | | |
|---|---|---|---|---|---|
| | Value | Std. Error | DF | t-value | p-value |
| (Intercept) | 2.7953518 | 0.09027788 | 1449 | 30.963861 | 0.0000 |
| Hapmap42294BTA69421G/A | −0.1477856 | 0.09022766 | 1449 | −1.637919 | 0.1017 |
| Hapmap42294BTA69421A/A | −0.2360581 | 0.11777214 | 1449 | −2.004363 | 0.0452 |
| Correlation: | | | | | |

| | | |
|---|---|---|
| | (Intr) | H42294BTA69421G |
| Hapmap42294BTA69421G/A | −0.587 | |
| Hapmap42294BTA69421A/A | −0.455 | 0.447 |

Standardized Within-Group Residuals:

| | | | | |
|---|---|---|---|---|
| Min | Q1 | Med | Q3 | Max |
| −1.8018736 | −0.7728889 | −0.0941675 | 0.6273581 | 4.7042023 |

Number of Observations: 1454
Number of Groups: 3

| | | | | |
|---|---|---|---|---|
| | numDF | denDF | F-value | p-value |
| (Intercept) | 1 | 1449 | 1448.445 | <.0001 |
| Hapmap42294BTA69421 | 2 | 1449 | 2.353 | 0.0954 |

Hapmap42294BTA69421 × BTB01553536
Linear mixed-effects model fit by REML
Data: vm

| | | |
|---|---|---|
| AIC | BIC | logLik |
| 5395.036 | 5421.436 | −2692.518 |

Random effects:
Formula: ~1|BTB01553536

| | | |
|---|---|---|
| | (Intercept) | Residual |
| StdDev: | 0.09478952 | 1.53678 |

Fixed effects: list(fixed)

| | | | | | |
|---|---|---|---|---|---|
| | Value | Std. Error | DF | t-value | p-value |
| (Intercept) | 2.7976060 | 0.08857855 | 1449 | 31.583335 | 0.0000 |
| Hapmap42294BTA69421G/A | −0.1521671 | 0.09021522 | 1449 | −1.686712 | 0.0919 |
| Hapmap42294BTA69421A/A | −0.2314614 | 0.11763026 | 1449 | −1.967703 | 0.0493 |
| Correlation: | | | | | |

| | | |
|---|---|---|
| | (Intr) | H42294BTA69421G |
| Hapmap42294BTA69421G/A | −0.594 | |
| Hapmap42294BTA69421A/A | −0.453 | 0.447 |

Standardized Within-Group Residuals:

| | | | | |
|---|---|---|---|---|
| Min | Q1 | Med | Q3 | Max |
| −1.8034362 | −0.7608448 | −0.1101335 | 0.6196408 | 4.7036767 |

Number of Observations: 1454
Number of Groups: 3

| | | | | |
|---|---|---|---|---|
| | numDF | denDF | F-value | p-value |
| (Intercept) | 1 | 1449 | 1521.1609 | <.0001 |
| Hapmap42294BTA69421 | 2 | 1449 | 2.3425 | 0.0964 |

Hapmap42294BTA69421 × HAPMAP53129RS29022984
Linear mixed-effects model fit by REML
Data: vm

| | | |
|---|---|---|
| AIC | BIC | logLik |
| 5390.451 | 5416.851 | −2690.226 |

Random effects:
Formula: ~1|HAPMAP53129RS29022984

| | | |
|---|---|---|
| | (Intercept) | Residual |
| StdDev: | 0.1739614 | 1.534102 |

Fixed effects: list(fixed)

| | | | | | |
|---|---|---|---|---|---|
| | Value | Std. Error | DF | t-value | p-value |
| (Intercept) | 2.7164192 | 0.13375606 | 1449 | 20.308756 | 0.0000 |
| Hapmap42294BTA69421G/A | −0.1490118 | 0.09004187 | 1449 | −1.654916 | 0.0982 |
| Hapmap42294BTA69421A/A | −0.2333802 | 0.11740317 | 1449 | −1.987852 | 0.0470 |
| Correlation: | | | | | |

| | | |
|---|---|---|
| | (Intr) | H42294BTA69421G |
| Hapmap42294BTA69421G/A | −0.390 | |
| Hapmap42294BTA69421A/A | −0.294 | 0.447 |

Standardized Within-Group Residuals:

| | | | | |
|---|---|---|---|---|
| Min | Q1 | Med | Q3 | Max |
| −1.7974360 | −0.7465753 | −0.1026331 | 0.6102986 | 4.7210367 |

Number of Observations: 1454
Number of Groups: 3

| | | | | |
|---|---|---|---|---|
| | numDF | denDF | F-value | p-value |
| (Intercept) | 1 | 1449 | 457.0622 | <.0001 |
| Hapmap42294BTA69421 | 2 | 1449 | 2.3426 | 0.0964 |

Hapmap42294BTA69421 × ARSBFGLNGS68110
Linear mixed-effects model fit by REML
Data: vm

| | | |
|---|---|---|
| AIC | BIC | logLik |
| 5358.182 | 5384.582 | −2674.091 |

Random effects:
Formula: ~1|ARSBFGLNGS68110

| | | |
|---|---|---|
| | (Intercept) | Residual |
| StdDev: | 0.3223662 | 1.515288 |

Fixed effects: list(fixed)

| | | | | | |
|---|---|---|---|---|---|
| | Value | Std. Error | DF | t-value | p-value |
| (Intercept) | 2.7710598 | 0.19884612 | 1449 | 13.935700 | 0.0000 |
| Hapmap42294BTA69421G/A | −0.1608494 | 0.08895469 | 1449 | −1.808218 | 0.0708 |
| Hapmap42294BTA69421A/A | −0.2763192 | 0.11617187 | 1449 | −2.378538 | 0.0175 |

Correlation:

| | | |
|---|---|---|
| | (Intr) | H42294BTA69421G |
| Hapmap42294BTA69421G/A | −0.260 | |
| Hapmap42294BTA69421A/A | −0.198 | 0.447 |

Standardized Within-Group Residuals:

| | | | | |
|---|---|---|---|---|
| Min | Q1 | Med | Q3 | Max |
| −2.0017454 | −0.7465294 | −0.1086502 | 0.6331232 | 4.5976604 |

Number of Observations: 1454
Number of Groups: 3

| | | | | |
|---|---|---|---|---|
| | numDF | denDF | F-value | p-value |
| (Intercept) | 1 | 1449 | 191.59086 | <.0001 |
| Hapmap42294BTA69421 | 2 | 1449 | 3.17471 | 0.0421 |

Hapmap42294BTA69421 × HAPMAP49592BTA38891
Linear mixed-effects model fit by REML
Data: vm

| | | |
|---|---|---|
| AIC | BIC | logLik |
| 5397.045 | 5423.445 | −2693.523 |

Random effects:
Formula: ~1|HAPMAP49592BTA38891

| | | |
|---|---|---|
| | (Intercept) | Residual |
| StdDev: | 0.04175297 | 1.538694 |

Fixed effects: list(fixed)

| | | | | | |
|---|---|---|---|---|---|
| | Value | Std. Error | DF | t-value | p-value |
| (Intercept) | 2.7933355 | 0.07525161 | 1449 | 37.11994 | 0.0000 |
| Hapmap42294BTA69421G/A | −0.1475831 | 0.09032713 | 1449 | −1.63387 | 0.1025 |
| Hapmap42294BTA69421A/A | −0.2264724 | 0.11773537 | 1449 | −1.92357 | 0.0546 |
| Correlation: | | | | | |

| | | |
|---|---|---|
| | (Intr) | H42294BTA69421G |
| Hapmap42294BTA69421G/A | −0.697 | |
| Hapmap42294BTA69421A/A | −0.539 | 0.447 |

Standardized Within-Group Residuals:

| | | | | |
|---|---|---|---|---|
| Min | Q1 | Med | Q3 | Max |
| −1.7631955 | −0.7574183 | −0.1037975 | 0.6110946 | 4.7532622 |

Number of Observations: 1454
Number of Groups: 3

| | | | | |
|---|---|---|---|---|
| | numDF | denDF | F-value | p-value |
| (Intercept) | 1 | 1449 | 2827.0141 | <.0001 |
| Hapmap42294BTA69421 | 2 | 1449 | 2.2245 | 0.1085 |

Hapmap42294BTA69421 × ARSBFGLNGS30157
Linear mixed-effects model fit by REML
Data: vm

| | | |
|---|---|---|
| AIC | BIC | logLik |
| 5392.923 | 5419.324 | −2691.462 |

Random effects:
Formula: ~1|ARSBFGLNGS30157

| | | |
|---|---|---|
| | (Intercept) | Residual |
| StdDev: | 0.1893257 | 1.535484 |

Fixed effects: list(fixed)

| | | | | | |
|---|---|---|---|---|---|
| | Value | Std. Error | DF | t-value | p-value |
| (Intercept) | 2.9007827 | 0.14807868 | 1449 | 19.589468 | 0.0000 |
| Hapmap42294BTA69421G/A | −0.1481278 | 0.09012976 | 1449 | −1.643495 | 0.1005 |
| Hapmap42294BTA69421A/A | −0.2265125 | 0.11749169 | 1449 | −1.927902 | 0.0541 |
| Correlation: | | | | | |

| | | |
|---|---|---|
| | (Intr) | H42294BTA69421G |
| Hapmap42294BTA69421G/A | −0.349 | |
| Hapmap42294BTA69421A/A | −0.267 | 0.447 |

Standardized Within-Group Residuals:

| | | | | |
|---|---|---|---|---|
| Min | Q1 | Med | Q3 | Max |
| −1.80195923 | −0.74432119 | −0.09306094 | 0.62332534 | 4.79139095 |

Number of Observations: 1454
Number of Groups: 3

| | | | | |
|---|---|---|---|---|
| | numDF | denDF | F-value | p-value |
| (Intercept) | 1 | 1449 | 411.8248 | <.0001 |
| Hapmap42294BTA69421 | 2 | 1449 | 2.2399 | 0.1068 |

Hapmap42294BTA69421 × HAPMAP30097BTC007678
Linear mixed-effects model fit by REML
Data: vm

| | | |
|---|---|---|
| AIC | BIC | logLik |
| 5397.078 | 5423.478 | −2693.539 |

Random effects:
Formula: ~1|HAPMAP30097BTC007678

| | | |
|---|---|---|
| | (Intercept) | Residual |
| StdDev: | 0.0001088537 | 1.538923 |

Fixed effects: list(fixed)

| | | | | | |
|---|---|---|---|---|---|
| | Value | Std. Error | DF | t-value | p-value |
| (Intercept) | 2.7939760 | 0.06896084 | 1449 | 40.51540 | 0.0000 |
| Hapmap42294BTA69421G/A | −0.1485736 | 0.09032328 | 1449 | −1.64491 | 0.1002 |
| Hapmap42294BTA69421A/A | −0.2266682 | 0.11774707 | 1449 | −1.92504 | 0.0544 |
| Correlation: | | | | | |

| | | |
|---|---|---|
| | (Intr) | H42294BTA69421G |
| Hapmap42294BTA69421G/A | −0.763 | |
| Hapmap42294BTA69421A/A | −0.586 | 0.447 |

Standardized Within-Group Residuals:

| | | | | |
|---|---|---|---|---|
| Min | Q1 | Med | Q3 | Max |
| −1.75055953 | −0.74428838 | −0.09448315 | 0.62030251 | 4.74749219 |

Number of Observations: 1454
Number of Groups: 3

| | | | | |
|---|---|---|---|---|
| | numDF | denDF | F-value | p-value |
| (Intercept) | 1 | 1449 | 4417.241 | <.0001 |
| Hapmap42294BTA69421 | 2 | 1449 | 2.237 | 0.1071 |

ARSBFGLBAC2384 × ARSBFGLNGS102860
Linear mixed-effects model fit by REML
Data: vm

| | | |
|---|---|---|
| AIC | BIC | logLik |
| 5395.387 | 5421.787 | −2692.693 |

Random effects:
Formula: ~1|ARSBFGLNGS102860

| | | |
|---|---|---|
| | (Intercept) | Residual |
| StdDev: | 0.1515687 | 1.536920 |

Fixed effects: list(fixed)

| | | | | | |
|---|---|---|---|---|---|
| | Value | Std. Error | DF | t-value | p-value |
| (Intercept) | 2.7449617 | 0.11541705 | 1449 | 23.782984 | 0.0000 |
| ARSBFGLBAC2384G/T | −0.1827260 | 0.08467632 | 1449 | −2.157935 | 0.0311 |
| ARSBFGLBAC2384T/T | −0.1730332 | 0.15123131 | 1449 | −1.144162 | 0.2527 |
| Correlation: | | | | | |

| | | |
|---|---|---|
| | (Intr) | ARSBFGLBAC2384G |
| ARSBFGLBAC2384G/T | −0.407 | |
| ARSBFGLBAC2384T/T | −0.233 | 0.306 |

Standardized Within-Group Residuals:

| | | | | |
|---|---|---|---|---|
| Min | Q1 | Med | Q3 | Max |
| −1.7946377 | −0.7679371 | −0.1141815 | 0.6119993 | 4.7738453 |

Number of Observations: 1454
Number of Groups: 3

| | | | | |
|---|---|---|---|---|
| | numDF | denDF | F-value | p-value |
| (Intercept) | 1 | 1449 | 635.2972 | <.0001 |
| ARSBFGLBAC2384 | 2 | 1449 | 2.4578 | 0.086 |

ARSBFGLBAC2384 × BFGLNGS119018
Linear mixed-effects model fit by REML
Data: vm

| | | |
|---|---|---|
| AIC | BIC | logLik |
| 5395.613 | 5422.013 | −2692.806 |

Random effects:
Formula: ~1|BFGLNGS119018

| | | |
|---|---|---|
| | (Intercept) | Residual |
| StdDev: | 0.07424423 | 1.537928 |

Fixed effects: list(fixed)

| | | | | | |
|---|---|---|---|---|---|
| | Value | Std. Error | DF | t-value | p-value |
| (Intercept) | 2.7622057 | 0.08293415 | 1449 | 33.30601 | 0.0000 |
| ARSBFGLBAC2384G/T | −0.1778997 | 0.08484312 | 1449 | −2.09681 | 0.0362 |
| ARSBFGLBAC2384T/T | −0.1615471 | 0.15113764 | 1449 | −1.06887 | 0.2853 |
| Correlation: | | | | | |

| | | |
|---|---|---|
| | (Intr) | ARSBFGLBAC2384G |
| ARSBFGLBAC2384G/T | −0.572 | |
| ARSBFGLBAC2384T/T | −0.319 | 0.306 |

Standardized Within-Group Residuals:

| | | | | |
|---|---|---|---|---|
| Min | Q1 | Med | Q3 | Max |
| −1.7599272 | −0.7549520 | −0.1343639 | 0.6315363 | 4.7423260 |

Number of Observations: 1454
Number of Groups: 3

| | | | | |
|---|---|---|---|---|
| | numDF | denDF | F-value | p-value |
| (Intercept) | 1 | 1449 | 1579.0787 | <.0001 |
| ARSBFGLBAC2384 | 2 | 1449 | 2.2989 | 0.1007 |

ARSBFGLBAC2384 × ARSBFGLBAC20850
Linear mixed-effects model fit by REML
Data: vm

| | | |
|---|---|---|
| AIC | BIC | logLik |
| 5396.16 | 5422.56 | −2693.08 |

Random effects:
Formula: ~1|ARSBFGLBAC20850

| | | |
|---|---|---|
| | (Intercept) | Residual |
| StdDev: | 9.544584e−05 | 1.538700 |

Fixed effects: list(fixed)

| | | | | | |
|---|---|---|---|---|---|
| | Value | Std. Error | DF | t-value | p-value |
| (Intercept) | 2.7879339 | 0.06255711 | 1449 | 44.56622 | 0.0000 |
| ARSBFGLBAC2384G/T | −0.1835140 | 0.08475582 | 1449 | −2.16521 | 0.0305 |
| ARSBFGLBAC2384T/T | −0.1655339 | 0.15117596 | 1449 | −1.09497 | 0.2737 |
| Correlation: | | | | | |

| | | |
|---|---|---|
| | (Intr) | ARSBFGLBAC2384G |
| ARSBFGLBAC2384G/T | −0.738 | |
| ARSBFGLBAC2384T/T | −0.414 | 0.305 |

Standardized Within-Group Residuals:

| | | | | |
|---|---|---|---|---|
| Min | Q1 | Med | Q3 | Max |
| −1.7468861 | −0.7720373 | −0.1221381 | 0.6246938 | 4.7521058 |

Number of Observations: 1454
Number of Groups: 3

| | | | | |
|---|---|---|---|---|
| | numDF | denDF | F-value | p-value |
| (Intercept) | 1 | 1449 | 4418.525 | <.0001 |
| ARSBFGLBAC2384 | 2 | 1449 | 2.448 | 0.0868 |

ARSBFGLBAC2384 × ARSBFGLNGS100843
Linear mixed-effects model fit by REML
Data: vm

| | | |
|---|---|---|
| AIC | BIC | logLik |
| 5394.609 | 5421.009 | −2692.305 |

Random effects:
Formula: ~1|ARSBFGLNGS100843

| | | |
|---|---|---|
| | (Intercept) | Residual |
| StdDev: | 0.1428355 | 1.537056 |

Fixed effects: list(fixed)

| | | | | | |
|---|---|---|---|---|---|
| | Value | Std. Error | DF | t-value | p-value |
| (Intercept) | 2.8773145 | 0.12188680 | 1449 | 23.606448 | 0.0000 |
| ARSBFGLBAC2384G/T | −0.1898233 | 0.08474093 | 1449 | −2.240042 | 0.0252 |
| ARSBFGLBAC2384T/T | −0.1796385 | 0.15122770 | 1449 | −1.187867 | 0.2351 |
| Correlation: | | | | | |

| | | |
|---|---|---|
| | (Intr) | ARSBFGLBAC2384G |
| ARSBFGLBAC2384G/T | −0.395 | |
| ARSBFGLBAC2384T/T | −0.232 | 0.307 |

Standardized Within-Group Residuals:

| | | | | |
|---|---|---|---|---|
| Min | Q1 | Med | Q3 | Max |
| −1.7351689 | −0.7642428 | −0.1153036 | 0.6069713 | 4.7707762 |

Number of Observations: 1454
Number of Groups: 3

| | | | | |
|---|---|---|---|---|
| | numDF | denDF | F-value | p-value |
| (Intercept) | 1 | 1449 | 618.5304 | <.0001 |
| ARSBFGLBAC2384 | 2 | 1449 | 2.6470 | 0.0712 |

ARSBFGLBAC2384 × ARSBFGLNGS97162
Linear mixed-effects model fit by REML
Data: vm

| | | |
|---|---|---|
| AIC | BIC | logLik |
| 5393.427 | 5419.827 | −2691.714 |

Random effects:
Formula: ~1|ARSBFGLNGS97162

| | | |
|---|---|---|
| | (Intercept) | Residual |
| StdDev: | 0.1420974 | 1.536271 |

Fixed effects: list(fixed)

| | | | | | |
|---|---|---|---|---|---|
| | Value | Std. Error | DF | t-value | p-value |
| (Intercept) | 2.8293317 | 0.11688976 | 1449 | 24.205129 | 0.0000 |
| ARSBFGLBAC2384G/T | −0.1498274 | 0.08613697 | 1449 | −1.739408 | 0.0822 |
| ARSBFGLBAC2384T/T | −0.1338311 | 0.15170395 | 1449 | −0.882186 | 0.3778 |
| Correlation: | | | | | |

| | | |
|---|---|---|
| | (Intr) | ARSBFGLBAC2384G |
| ARSBFGLBAC2384G/T | −0.375 | |
| ARSBFGLBAC2384T/T | −0.214 | 0.317 |

Standardized Within-Group Residuals:

| | | | | |
|---|---|---|---|---|
| Min | Q1 | Med | Q3 | Max |
| −1.8184886 | −0.7607483 | −0.1098213 | 0.6061984 | 4.8047899 |

Number of Observations: 1454
Number of Groups: 3

| | | | | |
|---|---|---|---|---|
| | numDF | denDF | F-value | p-value |
| (Intercept) | 1 | 1449 | 651.1676 | <.0001 |
| ARSBFGLBAC2384 | 2 | 1449 | 1.5734 | 0.2077 |

ARSBFGLBAC2384 × Hapmap42294BTA69421
Linear mixed-effects model fit by REML
Data: vm

| | | |
|---|---|---|
| AIC | BIC | logLik |
| 5395.24 | 5421.64 | −2692.62 |

Random effects:
Formula: ~1|Hapmap42294BTA69421

| | | |
|---|---|---|
| | (Intercept) | Residual |
| StdDev: | 0.08630119 | 1.537292 |

Fixed effects: list(fixed)

| | | | | | |
|---|---|---|---|---|---|
| | Value | Std. Error | DF | t-value | p-value |
| (Intercept) | 2.7828552 | 0.08063067 | 1449 | 34.51361 | 0.0000 |
| ARSBFGLBAC2384G/T | −0.1868477 | 0.08476046 | 1449 | −2.20442 | 0.0277 |
| ARSBFGLBAC2384T/T | −0.1593574 | 0.15108799 | 1449 | −1.05473 | 0.2917 |
| Correlation: | | | | | |

| | | |
|---|---|---|
| | (Intr) | ARSBFGLBAC2384G |
| ARSBFGLBAC2384G/T | −0.566 | |
| ARSBFGLBAC2384T/T | −0.321 | 0.305 |

Standardized Within-Group Residuals:

| | | | | |
|---|---|---|---|---|
| Min | Q1 | Med | Q3 | Max |
| −1.7925126 | −0.7656261 | −0.1097828 | 0.6089674 | 4.7124308 |

Number of Observations: 1454
Number of Groups: 3

| | | | | |
|---|---|---|---|---|
| | numDF | denDF | F-value | p-value |
| (Intercept) | 1 | 1449 | 1682.2042 | <.0001 |
| ARSBFGLBAC2384 | 2 | 1449 | 2.5107 | 0.0816 |

ARSBFGLBAC2384 × ARSBFGLBAC2384
Linear mixed-effects model fit by REML
Data: vm

| | | |
|---|---|---|
| AIC | BIC | logLik |
| 5396.16 | 5422.56 | −2693.08 |

Random effects:
Formula: ~1|ARSBFGLBAC2384

| | | |
|---|---|---|
| | (Intercept) | Residual |
| StdDev: | 0.1863808 | 1.538700 |

Fixed effects: list(fixed)

| | | | | | |
|---|---|---|---|---|---|
| | Value | Std. Error | DF | t-value | p-value |
| (Intercept) | 2.7879339 | 0.1965990 | 1451 | 14.180811 | 0 |
| ARSBFGLBAC2384G/T | −0.1835140 | 0.2768739 | 0 | −0.662807 | NaN |
| ARSBFGLBAC2384T/T | −0.1655339 | 0.3038581 | 0 | −0.544774 | NaN |
| Correlation: | | | | | |

| | | |
|---|---|---|
| | (Intr) | ARSBFGLBAC2384G |
| ARSBFGLBAC2384G/T | −0.710 | |
| ARSBFGLBAC2384T/T | −0.647 | 0.459 |

Standardized Within-Group Residuals:

| | | | | |
|---|---|---|---|---|
| Min | Q1 | Med | Q3 | Max |
| −1.7468861 | −0.7720373 | −0.1221381 | 0.6246938 | 4.7521058 |

Number of Observations: 1454
Number of Groups: 3

| | | | | |
|---|---|---|---|---|
| | numDF | denDF | F-value | p-value |
| (Intercept) | 1 | 1451 | 507.1596 | <.0001 |
| ARSBFGLBAC2384 | 2 | 0 | 0.2562 | NaN |

ARSBFGLBAC2384 × BTB01553536
Linear mixed-effects model fit by REML
Data: vm

| | | |
|---|---|---|
| AIC | BIC | logLik |
| 5394.647 | 5421.048 | −2692.324 |

Random effects:
Formula: ~1|BTB01553536

| | | |
|---|---|---|
| | (Intercept) | Residual |
| StdDev: | 0.08600099 | 1.536964 |

Fixed effects: list(fixed)

| | | | | | |
|---|---|---|---|---|---|
| | Value | Std. Error | DF | t-value | p-value |
| (Intercept) | 2.7863539 | 0.08065064 | 1449 | 34.54844 | 0.0000 |
| ARSBFGLBAC2384G/T | −0.1761300 | 0.08478743 | 1449 | −2.07731 | 0.0379 |
| ARSBFGLBAC2384T/T | −0.1691431 | 0.15104327 | 1449 | −1.11983 | 0.2630 |
| Correlation: | | | | | |

| | | |
|---|---|---|
| | (Intr) | ARSBFGLBAC2384G |
| ARSBFGLBAC2384G/T | −0.575 | |
| ARSBFGLBAC2384T/T | −0.323 | 0.305 |

Standardized Within-Group Residuals:

| | | | | |
|---|---|---|---|---|
| Min | Q1 | Med | Q3 | Max |
| −1.7518328 | −0.7473229 | −0.1252496 | 0.6234245 | 4.7179965 |

Number of Observations: 1454
Number of Groups: 3

| | | | | |
|---|---|---|---|---|
| | numDF | denDF | F-value | p-value |
| (Intercept) | 1 | 1449 | 1716.4921 | <.0001 |
| ARSBFGLBAC2384 | 2 | 1449 | 2.2882 | 0.1018 |

ARSBFGLBAC2384 × HAPMAP53129RS29022984
Linear mixed-effects model fit by REML
Data: vm

| | | |
|---|---|---|
| AIC | BIC | logLik |
| 5390.061 | 5416.461 | −2690.031 |

Random effects:
Formula: ~1|HAPMAP53129RS29022984

| | | |
|---|---|---|
| | (Intercept) | Residual |
| StdDev: | 0.1688692 | 1.534198 |

Fixed effects: list(fixed)

| | | | | | |
|---|---|---|---|---|---|
| | Value | Std. Error | DF | t-value | p-value |
| (Intercept) | 2.7083479 | 0.12837049 | 1449 | 21.097901 | 0.0000 |
| ARSBFGLBAC2384G/T | −0.1768097 | 0.08454132 | 1449 | −2.091400 | 0.0367 |
| ARSBFGLBAC2384T/T | −0.1609768 | 0.15074193 | 1449 | −1.067897 | 0.2857 |
| Correlation: | | | | | |

| | | |
|---|---|---|
| | (Intr) | ARSBFGLBAC2384G |
| ARSBFGLBAC2384G/T | −0.361 | |
| ARSBFGLBAC2384T/T | −0.203 | 0.306 |

Standardized Within-Group Residuals:

| | | | | |
|---|---|---|---|---|
| Min | Q1 | Med | Q3 | Max |
| −1.7885114 | −0.7456213 | −0.1089305 | 0.6080564 | 4.7295518 |

Number of Observations: 1454
Number of Groups: 3

| | | | | |
|---|---|---|---|---|
| | numDF | denDF | F-value | p-value |
| (Intercept) | 1 | 1449 | 479.1100 | <.0001 |
| ARSBFGLBAC2384 | 2 | 1449 | 2.2884 | 0.1018 |

ARSBFGLBAC2384 × ARSBFGLNGS68110
Linear mixed-effects model fit by REML
Data: vm

| | | |
|---|---|---|
| AIC | BIC | logLik |
| 5362.615 | 5389.015 | −2676.308 |

Random effects:
Formula: ~1|ARSBFGLNGS68110

| | | |
|---|---|---|
| | (Intercept) | Residual |
| StdDev: | 0.3047465 | 1.517993 |

Fixed effects: list(fixed)

| | | | | | |
|---|---|---|---|---|---|
| | Value | Std. Error | DF | t-value | p-value |
| (Intercept) | 2.7036181 | 0.18789661 | 1449 | 14.388861 | 0.0000 |
| ARSBFGLBAC2384G/T | −0.0988124 | 0.08476775 | 1449 | −1.165683 | 0.2439 |
| ARSBFGLBAC2384T/T | −0.0763904 | 0.14985523 | 1449 | −0.509761 | 0.6103 |
| Correlation: | | | | | |

| | | |
|---|---|---|
| | (Intr) | ARSBFGLBAC2384G |
| ARSBFGLBAC2384G/T | −0.255 | |
| ARSBFGLBAC2384T/T | −0.144 | 0.316 |

Standardized Within-Group Residuals:

| | | | | |
|---|---|---|---|---|
| Min | Q1 | Med | Q3 | Max |
| −1.93999320 | −0.73222299 | −0.09545253 | 0.62840607 | 4.64765204 |

Number of Observations: 1454
Number of Groups: 3

| | | | | |
|---|---|---|---|---|
| | numDF | denDF | F-value | p-value |
| (Intercept) | 1 | 1449 | 213.10564 | <.0001 |
| ARSBFGLBAC2384 | 2 | 1449 | 0.69054 | 0.5015 |

ARSBFGLBAC2384 × HAPMAP49592BTA38891
Linear mixed-effects model fit by REML
Data: vm

| | | |
|---|---|---|
| AIC | BIC | logLik |
| 5396.045 | 5422.445 | −2693.023 |

Random effects:
Formula: ~1|HAPMAP49592BTA38891

| | | |
|---|---|---|
| | (Intercept) | Residual |
| StdDev: | 0.06413626 | 1.538217 |

Fixed effects: list(fixed)

| | | | | | |
|---|---|---|---|---|---|
| | Value | Std. Error | DF | t-value | p-value |
| (Intercept) | 2.7857832 | 0.07751788 | 1449 | 35.93730 | 0.0000 |
| ARSBFGLBAC2384G/T | −0.1839824 | 0.08473656 | 1449 | −2.17123 | 0.0301 |
| ARSBFGLBAC2384T/T | −0.1702075 | 0.15126702 | 1449 | −1.12521 | 0.2607 |
| Correlation: | | | | | |

| | | |
|---|---|---|
| | (Intr) | ARSBFGLBAC2384G |
| ARSBFGLBAC2384G/T | −0.599 | |
| ARSBFGLBAC2384T/T | −0.342 | 0.306 |

Standardized Within-Group Residuals:

| | | | | |
|---|---|---|---|---|
| Min | Q1 | Med | Q3 | Max |
| −1.7714243 | −0.7745399 | −0.1140235 | 0.6112147 | 4.7617508 |

Number of Observations: 1454
Number of Groups: 3

| | | | | |
|---|---|---|---|---|
| | numDF | denDF | F-value | p-value |
| (Intercept) | 1 | 1449 | 1948.2549 | <.0001 |
| ARSBFGLBAC2384 | 2 | 1449 | 2.4746 | 0.0845 |

ARSBFGLBAC2384 × ARSBFGLNGS30157
Linear mixed-effects model fit by REML
Data: vm

| | | |
|---|---|---|
| AIC | BIC | logLik |
| 5391.393 | 5417.793 | −2690.696 |

Random effects:
Formula: ~1|ARSBFGLNGS30157

| | | |
|---|---|---|
| | (Intercept) | Residual |
| StdDev: | 0.1969388 | 1.534897 |

Fixed effects: list(fixed)

| | | | | | |
|---|---|---|---|---|---|
| | Value | Std. Error | DF | t-value | p-value |
| (Intercept) | 2.9063735 | 0.14991169 | 1449 | 19.387237 | 0.0000 |
| ARSBFGLBAC2384G/T | −0.1916559 | 0.08465849 | 1449 | −2.263872 | 0.0237 |
| ARSBFGLBAC2384T/T | −0.1944462 | 0.15123260 | 1449 | −1.285743 | 0.1987 |
| Correlation: | | | | | |

| | | |
|---|---|---|
| | (Intr) | ARSBFGLBAC2384G |
| ARSBFGLBAC2384G/T | −0.302 | |
| ARSBFGLBAC2384T/T | −0.183 | 0.308 |

Standardized Within-Group Residuals:

| | | | | |
|---|---|---|---|---|
| Min | Q1 | Med | Q3 | Max |
| −1.87550771 | −0.74843343 | −0.09692393 | 0.61973652 | 4.79483353 |

Number of Observations: 1454
Number of Groups: 3

| | | | | |
|---|---|---|---|---|
| | numDF | denDF | F-value | p-value |
| (Intercept) | 1 | 1449 | 386.3754 | <.0001 |
| ARSBFGLBAC2384 | 2 | 1449 | 2.7544 | 0.064 |

ARSBFGLBAC2384 × HAPMAP30097BTC007678
Linear mixed-effects model fit by REML
Data: vm

| | | |
|---|---|---|
| AIC | BIC | logLik |
| 5396.16 | 5422.56 | −2693.08 |

Random effects:
Formula: ~1|HAPMAP30097BTC007678

| | | |
|---|---|---|
| | (Intercept) | Residual |
| StdDev: | 0.0001250114 | 1.538700 |

Fixed effects: list(fixed)

| | | | | | |
|---|---|---|---|---|---|
| | Value | Std. Error | DF | t-value | p-value |
| (Intercept) | 2.7879340 | 0.06255715 | 1449 | 44.56619 | 0.0000 |
| ARSBFGLBAC2384G/T | −0.1835140 | 0.08475582 | 1449 | −2.16521 | 0.0305 |
| ARSBFGLBAC2384T/T | −0.1655339 | 0.15117596 | 1449 | −1.09497 | 0.2737 |
| Correlation: | | | | | |

| | | |
|---|---|---|
| | (Intr) | ARSBFGLBAC2384G |
| ARSBFGLBAC2384G/T | −0.738 | |
| ARSBFGLBAC2384T/T | −0.414 | 0.305 |

Standardized Within-Group Residuals:

| | | | | |
|---|---|---|---|---|
| Min | Q1 | Med | Q3 | Max |
| −1.7468861 | −0.7720373 | −0.1221381 | 0.6246938 | 4.7521059 |

Number of Observations: 1454
Number of Groups: 3

| | | | | |
|---|---|---|---|---|
| | numDF | denDF | F-value | p-value |
| (Intercept) | 1 | 1449 | 4418.513 | <.0001 |
| ARSBFGLBAC2384 | 2 | 1449 | 2.448 | 0.0868 |

BTB01553536 × ARSBFGLNGS102860
Linear mixed-effects model fit by REML
Data: vm

| | | |
|---|---|---|
| AIC | BIC | logLik |
| 5395.563 | 5421.963 | −2692.782 |

Random effects:
Formula: −1|ARSBFGLNGS102860

| | | |
|---|---|---|
| | (Intercept) | Residual |
| StdDev: | 0.1428732 | 1.536794 |

Fixed effects: list(fixed)

| | | | | | |
|---|---|---|---|---|---|
| | Value | Std. Error | DF | t-value | p-value |
| (Intercept) | 2.5357345 | 0.11094951 | 1449 | 22.854851 | 0.0000 |
| BTB01553536T/C | 0.2066238 | 0.08981577 | 1449 | 2.300529 | 0.0216 |
| BTB01553536C/C | 0.1171516 | 0.11140233 | 1449 | 1.051608 | 0.2932 |
| Correlation: | | | | | |

| | | |
|---|---|---|
| | (Intr) | BTB01553536T |
| BTB01553536T/C | −0.399 | |
| BTB01553536C/C | −0.314 | 0.398 |

Standardized Within-Group Residuals:

| | | | | |
|---|---|---|---|---|
| Min | Q1 | Med | Q3 | Max |
| −1.7324039 | −0.7520358 | −0.1056406 | 0.6144454 | 4.7746493 |

Number of Observations: 1454
Number of Groups: 3

| | | | | |
|---|---|---|---|---|
| | numDF | denDF | F-value | p-value |
| (Intercept) | 1 | 1449 | 697.6257 | <.0001 |
| BTB01553536 | 2 | 1449 | 2.6573 | 0.0705 |

BTB01553536 × BFGLNGS119018
Linear mixed-effects model fit by REML
Data: vm

| | | |
|---|---|---|
| AIC | BIC | logLik |
| 5395.043 | 5421.443 | −2692.521 |

Random effects:
Formula: ~1|BFGLNGS119018

| | | |
|---|---|---|
| | (Intercept) | Residual |
| StdDev: | 0.09011135 | 1.537177 |

Fixed effects: list(fixed)

| | | | | | |
|---|---|---|---|---|---|
| | Value | Std. Error | DF | t-value | p-value |
| (Intercept) | 2.5424100 | 0.09006673 | 1449 | 28.228070 | 0.0000 |
| BTB01553536T/C | 0.2146705 | 0.08979144 | 1449 | 2.390768 | 0.0169 |
| BTB01553536C/C | 0.1172670 | 0.11136500 | 1449 | 1.052997 | 0.2925 |
| Correlation: | | | | | |

| | | |
|---|---|---|
| | (Intr) | BTB01553536T |
| BTB01553536T/C | −0.501 | |
| BTB01553536C/C | −0.403 | 0.399 |

Standardized Within-Group Residuals:

| | | | | |
|---|---|---|---|---|
| Min | Q1 | Med | Q3 | Max |
| −1.7678748 | −0.7270060 | −0.1319734 | 0.6374453 | 4.7375556 |

Number of Observations: 1454
Number of Groups: 3

| | | | | |
|---|---|---|---|---|
| | numDF | denDF | F-value | p-value |
| (Intercept) | 1 | 1449 | 1238.5535 | <.0001 |
| BTB01553536 | 2 | 1449 | 2.8636 | 0.0574 |

BTB01553536 × ARSBFGLBAC20850
Linear mixed-effects model fit by REML
Data: vm

| | | |
|---|---|---|
| AIC | BIC | logLik |
| 5396.059 | 5422.459 | −2693.030 |

Random effects:
Formula: ~1|ARSBFGLBAC20850

| | | |
|---|---|---|
| | (Intercept) | Residual |
| StdDev: | 9.884489e−05 | 1.538345 |

Fixed effects: list(fixed)

| | | | | | |
|---|---|---|---|---|---|
| | Value | Std. Error | DF | t-value | p-value |
| (Intercept) | 2.5753378 | 0.06322567 | 1449 | 40.73247 | 0.0000 |
| BTB01553536T/C | 0.2117534 | 0.08983673 | 1449 | 2.35709 | 0.0186 |
| BTB01553536C/C | 0.1157654 | 0.11144150 | 1449 | 1.03880 | 0.2991 |
| Correlation: | | | | | |

| | | |
|---|---|---|
| | (Intr) | BTB01553536T |
| BTB01553536T/C | −0.704 | |
| BTB01553536C/C | −0.567 | 0.399 |

Standardized Within-Group Residuals:

| | | | | |
|---|---|---|---|---|
| Min | Q1 | Med | Q3 | Max |
| −1.7467412 | −0.7640273 | −0.1216185 | 0.6010758 | 4.7537497 |

Number of Observations: 1454
Number of Groups: 3

| | | | | |
|---|---|---|---|---|
| | numDF | denDF | F-value | p-value |
| (Intercept) | 1 | 1449 | 4420.562 | <.0001 |
| BTB01553536 | 2 | 1449 | 2.784 | 0.0621 |

BTB01553536 × ARSBFGLNGS100843
Linear mixed-effects model fit by REML
Data: vm

| | | |
|---|---|---|
| AIC | BIC | logLik |
| 5394.99 | 5421.39 | −2692.495 |

Random effects:
Formula: ~1|ARSBFGLNGS100843

| | | |
|---|---|---|
| | (Intercept) | Residual |
| StdDev: | 0.1267431 | 1.537064 |

Fixed effects: list(fixed)

| | | | | | |
|---|---|---|---|---|---|
| | Value | Std. Error | DF | t-value | p-value |
| (Intercept) | 2.6486404 | 0.11251676 | 1449 | 23.539964 | 0.0000 |
| BTB01553536T/C | 0.2098300 | 0.08979809 | 1449 | 2.336687 | 0.0196 |
| BTB01553536C/C | 0.1148843 | 0.11135937 | 1449 | 1.031653 | 0.3024 |
| Correlation: | | | | | |

| | | |
|---|---|---|
| | (Intr) | BTB01553536T |
| BTB01553536T/C | −0.393 | |
| BTB01553536C/C | −0.317 | 0.399 |

Standardized Within-Group Residuals:

| | | | | |
|---|---|---|---|---|
| Min | Q1 | Med | Q3 | Max |
| −1.7649419 | −0.7513471 | −0.1287805 | 0.6148942 | 4.7722819 |

Number of Observations: 1454
Number of Groups: 3

| | | | | |
|---|---|---|---|---|
| | numDF | denDF | F-value | p-value |
| (Intercept) | 1 | 1449 | 735.2994 | <.0001 |
| BTB01553536 | 2 | 1449 | 2.7358 | 0.0652 |

BTB01553536 × ARSBFGLNGS97162
Linear mixed-effects model fit by REML
Data: vm

| | | |
|---|---|---|
| AIC | BIC | logLik |
| 5392.318 | 5418.718 | −2691.159 |

Random effects:
Formula: ~1|ARSBFGLNGS97162

| | | |
|---|---|---|
| | (Intercept) | Residual |
| StdDev: | 0.1555918 | 1.535274 |

Fixed effects: list(fixed)

| | | | | | |
|---|---|---|---|---|---|
| | Value | Std. Error | DF | t-value | p-value |
| (Intercept) | 2.6513276 | 0.12650859 | 1449 | 20.957688 | 0.0000 |
| BTB01553536T/C | 0.1973759 | 0.08985566 | 1449 | 2.196588 | 0.0282 |
| BTB01553536C/C | 0.1044186 | 0.11132189 | 1449 | 0.937988 | 0.3484 |
| Correlation: | | | | | |

| | | |
|---|---|---|
| | (Intr) | BTB01553536T |
| BTB01553536T/C | −0.366 | |
| BTB01553536C/C | −0.290 | 0.401 |

Standardized Within-Group Residuals:

| | | | | |
|---|---|---|---|---|
| Min | Q1 | Med | Q3 | Max |
| −1.7773579 | −0.7341428 | −0.1461057 | 0.6336915 | 4.8040385 |

Number of Observations: 1454
Number of Groups: 3

| | | | | |
|---|---|---|---|---|
| | numDF | denDF | F-value | p-value |
| (Intercept) | 1 | 1449 | 563.1603 | <.0001 |
| BTB01553536 | 2 | 1449 | 2.4145 | 0.0898 |

BTB01553536 × Hapmap42294BTA69421
Linear mixed-effects model fit by REML
Data: vm

| | | |
|---|---|---|
| AIC | BIC | logLik |
| 5395.093 | 5421.493 | −2692.546 |

Random effects:
Formula: ~1|Hapmap42294BTA69421

| | | |
|---|---|---|
| | (Intercept) | Residual |
| StdDev: | 0.08607573 | 1.536915 |

Fixed effects: list(fixed)

| | | | | | |
|---|---|---|---|---|---|
| | Value | Std. Error | DF | t-value | p-value |
| (Intercept) | 2.5689178 | 0.08128849 | 1449 | 31.602479 | 0.0000 |
| BTB01553536T/C | 0.2149684 | 0.08977466 | 1449 | 2.394534 | 0.0168 |
| BTB01553536C/C | 0.1154169 | 0.11135200 | 1449 | 1.036505 | 0.3001 |
| Correlation: | | | | | |

| | | |
|---|---|---|
| | (Intr) | BTB01553536T |
| BTB01553536T/C | −0.548 | |
| BTB01553536C/C | −0.438 | 0.399 |

Standardized Within-Group Residuals:

| | | | | |
|---|---|---|---|---|
| Min | Q1 | Med | Q3 | Max |
| −1.7940948 | −0.7530483 | −0.1048353 | 0.6206234 | 4.7124459 |

Number of Observations: 1454
Number of Groups: 3

| | | | | |
|---|---|---|---|---|
| | numDF | denDF | F-value | p-value |
| (Intercept) | 1 | 1449 | 1687.8366 | <.0001 |
| BTB01553536 | 2 | 1449 | 2.8708 | 0.057 |

BTB01553536 × ARSBFGLBAC2384
Linear mixed-effects model fit by REML
Data: vm

| | | |
|---|---|---|
| AIC | BIC | logLik |
| 5394.76 | 5421.16 | −2692.38 |

Random effects:
Formula: ~1|ARSBFGLBAC2384

| | | |
|---|---|---|
| | (Intercept) | Residual |
| StdDev: | 0.0831477 | 1.536874 |

Fixed effects: list(fixed)

| | | | | | |
|---|---|---|---|---|---|
| | Value | Std. Error | DF | t-value | p-value |
| (Intercept) | 2.5756653 | 0.08235763 | 1449 | 31.274156 | 0.0000 |
| BTB01553536T/C | 0.2048425 | 0.08988570 | 1449 | 2.278922 | 0.0228 |
| BTB01553536C/C | 0.1169138 | 0.11135244 | 1449 | 1.049944 | 0.2939 |
| Correlation: | | | | | |

| | | |
|---|---|---|
| | (Intr) | BTB01553536T |
| BTB01553536T/C | −0.547 | |
| BTB01553536C/C | −0.440 | 0.399 |

Standardized Within-Group Residuals:

| | | | | |
|---|---|---|---|---|
| Min | Q1 | Med | Q3 | Max |
| −1.7321649 | −0.7425470 | −0.1028808 | 0.6211099 | 4.7199429 |

Number of Observations: 1454
Number of Groups: 3

| | | | | |
|---|---|---|---|---|
| | numDF | denDF | F-value | p-value |
| (Intercept) | 1 | 1449 | 1649.7678 | <.0001 |
| BTB01553536 | 2 | 1449 | 2.6086 | 0.074 |

BTB01553536 × BTB01553536
Linear mixed-effects model fit by REML
Data: vm

| | | |
|---|---|---|
| AIC | BIC | logLik |
| 5396.059 | 5422.459 | −2693.030 |

Random effects:
Formula: ~1|BTB01553536

| | | |
|---|---|---|
| | (Intercept) | Residual |
| StdDev: | 0.1863378 | 1.538345 |

Fixed effects: list(fixed)

| | | | | | |
|---|---|---|---|---|---|
| | Value | Std. Error | DF | t-value | p-value |
| (Intercept) | 2.5753378 | 0.1967721 | 1451 | 13.087922 | 0 |
| BTB01553536T/C | 0.2117534 | 0.2784137 | 0 | 0.760571 | NaN |
| BTB01553536C/C | 0.1157654 | 0.2861167 | 0 | 0.404609 | NaN |
| Correlation: | | | | | |

| | | |
|---|---|---|
| | (Intr) | BTB01553536T |
| BTB01553536T/C | −0.707 | |
| BTB01553536C/C | −0.688 | 0.486 |

Standardized Within-Group Residuals:

| | | | | |
|---|---|---|---|---|
| Min | Q1 | Med | Q3 | Max |
| −1.7467412 | −0.7640273 | −0.1216185 | 0.6010758 | 4.7537497 |

Number of Observations: 1454
Number of Groups: 3

| | | | | |
|---|---|---|---|---|
| | numDF | denDF | F-value | p-value |
| (Intercept) | 1 | 1451 | 538.802 | <.0001 |
| BTB01553536 | 2 | 0 | 0.290 | NaN |

BTB01553536 × HAPMAP53129RS29022984
Linear mixed-effects model fit by REML
Data: vm

| | | |
|---|---|---|
| AIC | BIC | logLik |
| 5389.761 | 5416.161 | −2689.880 |

Random effects:
Formula: ~1|HAPMAP53129RS29022984

| | | |
|---|---|---|
| | (Intercept) | Residual |
| StdDev: | 0.1708219 | 1.533724 |

Fixed effects: list(fixed)

| | | | | | |
|---|---|---|---|---|---|
| | Value | Std. Error | DF | t-value | p-value |
| (Intercept) | 2.5018693 | 0.1294533 | 1449 | 19.326418 | 0.0000 |
| BTB01553536T/C | 0.2090514 | 0.0895725 | 1449 | 2.333879 | 0.0197 |
| BTB01553536C/C | 0.1041436 | 0.1111789 | 1449 | 0.936720 | 0.3491 |
| Correlation: | | | | | |

| | | |
|---|---|---|
| | (Intr) | BTB01553536T |
| BTB01553536T/C | −0.343 | |
| BTB01553536C/C | −0.272 | 0.399 |

Standardized Within-Group Residuals:

| | | | | |
|---|---|---|---|---|
| Min | Q1 | Med | Q3 | Max |
| −1.79193217 | −0.74281803 | −0.09671177 | 0.62049686 | 4.72814629 |

Number of Observations: 1454
Number of Groups: 3

| | | | | |
|---|---|---|---|---|
| | numDF | denDF | F-value | p-value |
| (Intercept) | 1 | 1449 | 470.5242 | <.0001 |
| BTB01553536 | 2 | 1449 | 2.7235 | 0.066 |

BTB01553536 × ARSBFGLNGS68110
Linear mixed-effects model fit by REML
Data: vm

| | | |
|---|---|---|
| AIC | BIC | logLik |
| 5360.14 | 5386.54 | −2675.07 |

Random effects:
Formula: ~1|ARSBFGLNGS68110

| | | |
|---|---|---|
| | (Intercept) | Residual |
| StdDev: | 0.3100581 | 1.516352 |

Fixed effects: list(fixed)

| | | | | | |
|---|---|---|---|---|---|
| | Value | Std. Error | DF | t-value | p-value |
| (Intercept) | 2.5497445 | 0.19025265 | 1449 | 13.401887 | 0.0000 |
| BTB01553536T/C | 0.1854537 | 0.08866679 | 1449 | 2.091580 | 0.0366 |
| BTB01553536C/C | 0.1199674 | 0.10985180 | 1449 | 1.092084 | 0.2750 |
| Correlation: | | | | | |

| | | |
|---|---|---|
| | (Intr) | BTB01553536T |
| BTB01553536T/C | −0.227 | |
| BTB01553536C/C | −0.186 | 0.399 |

Standardized Within-Group Residuals:

| | | | | |
|---|---|---|---|---|
| Min | Q1 | Med | Q3 | Max |
| −1.9671586 | −0.7147143 | −0.1115916 | 0.5941763 | 4.6276168 |

Number of Observations: 1454
Number of Groups: 3

| | | | | |
|---|---|---|---|---|
| | numDF | denDF | F-value | p-value |
| (Intercept) | 1 | 1449 | 206.27195 | <.0001 |
| BTB01553536 | 2 | 1449 | 2.22703 | 0.1082 |

BTB01553536 × HAPMAP49592BTA38891
Linear mixed-effects model fit by REML
Data: vm

| | | |
|---|---|---|
| AIC | BIC | logLik |
| 5396.019 | 5422.419 | −2693.010 |

Random effects:
Formula: ~1|HAPMAP49592BTA38891

| | | |
|---|---|---|
| | (Intercept) | Residual |
| StdDev: | 0.04508659 | 1.538084 |

Fixed effects: list(fixed)

| | | | | | |
|---|---|---|---|---|---|
| | Value | Std. Error | DF | t-value | p-value |
| (Intercept) | 2.5746302 | 0.07100732 | 1449 | 36.25866 | 0.0000 |
| BTB01553536T/C | 0.2111874 | 0.08982521 | 1449 | 2.35109 | 0.0189 |
| BTB01553536C/C | 0.1178379 | 0.11148601 | 1449 | 1.05698 | 0.2907 |
| Correlation: | | | | | |

| | | |
|---|---|---|
| | (Intr) | BTB01553536T |
| BTB01553536T/C | −0.627 | |
| BTB01553536C/C | −0.500 | 0.399 |

Standardized Within-Group Residuals:

| | | | | |
|---|---|---|---|---|
| Min | Q1 | Med | Q3 | Max |
| −1.7413100 | −0.7587970 | −0.1159110 | 0.6065382 | 4.7602862 |

Number of Observations: 1454
Number of Groups: 3

| | | | | |
|---|---|---|---|---|
| | numDF | denDF | F-value | p-value |
| (Intercept) | 1 | 1449 | 2676.2692 | <.0001 |
| BTB01553536 | 2 | 1449 | 2.7723 | 0.0628 |

BTB01553536 × ARSBFGLNGS30157
Linear mixed-effects model fit by REML
Data: vm

| | | |
|---|---|---|
| AIC | BIC | logLik |
| 5391.336 | 5417.736 | −2690.668 |

Random effects:
Formula: ~1|ARSBFGLNGS30157

| | | |
|---|---|---|
| | (Intercept) | Residual |
| StdDev: | 0.2066252 | 1.534512 |

Fixed effects: list(fixed)

| | | | | | |
|---|---|---|---|---|---|
| | Value | Std. Error | DF | t-value | p-value |
| (Intercept) | 2.6870857 | 0.1553489 | 1449 | 17.297107 | 0.0000 |
| BTB01553536T/C | 0.2204139 | 0.0896762 | 1449 | 2.457886 | 0.0141 |
| BTB01553536C/C | 0.1418535 | 0.1116063 | 1449 | 1.271016 | 0.2039 |
| Correlation: | | | | | |

| | | |
|---|---|---|
| | (Intr) | BTB01553536T |
| BTB01553536T/C | −0.281 | |
| BTB01553536C/C | −0.214 | 0.401 |

Standardized Within-Group Residuals:

| | | | | |
|---|---|---|---|---|
| Min | Q1 | Med | Q3 | Max |
| −1.8712307 | −0.7383552 | −0.1385463 | 0.6184299 | 4.8008653 |

Number of Observations: 1454
Number of Groups: 3

| | | | | |
|---|---|---|---|---|
| | numDF | denDF | F-value | p-value |
| (Intercept) | 1 | 1449 | 357.3572 | <.0001 |
| BTB01553536 | 2 | 1449 | 3.0693 | 0.0468 |

BTB01553536 × HAPMAP30097BTC007678
Linear mixed-effects model fit by REML
Data: vm

| | | |
|---|---|---|
| AIC | BIC | logLik |
| 5396.059 | 5422.459 | −2693.030 |

Random effects:
Formula: ~1|HAPMAP30097BTC007678

| | | |
|---|---|---|
| | (Intercept) | Residual |
| StdDev: | 0.0001096693 | 1.538345 |

Fixed effects: list(fixed)

| | | | | | |
|---|---|---|---|---|---|
| | Value | Std. Error | DF | t-value | p-value |
| (Intercept) | 2.5753379 | 0.06322567 | 1449 | 40.73247 | 0.0000 |
| BTB01553536T/C | 0.2117534 | 0.08983673 | 1449 | 2.35709 | 0.0186 |
| BTB01553536C/C | 0.1157654 | 0.11144150 | 1449 | 1.03880 | 0.2991 |
| Correlation: | | | | | |

| | | |
|---|---|---|
| | (Intr) | BTB01553536T |
| BTB01553536T/C | −0.704 | |
| BTB01553536C/C | −0.567 | 0.399 |

Standardized Within-Group Residuals:

| | | | | |
|---|---|---|---|---|
| Min | Q1 | Med | Q3 | Max |
| −1.7467413 | −0.7640273 | −0.1216185 | 0.6010758 | 4.7537497 |

Number of Observations: 1454
Number of Groups: 3

| | | | | |
|---|---|---|---|---|
| | numDF | denDF | F-value | p-value |
| (Intercept) | 1 | 1449 | 4420.558 | <.0001 |
| BTB01553536 | 2 | 1449 | 2.784 | 0.0621 |

HAPMAP53129RS29022984 × ARSBFGLNGS102860
Linear mixed-effects model fit by REML
Data: vm

| | | |
|---|---|---|
| AIC | BIC | logLik |
| 5388.45 | 5414.85 | −2689.225 |

Random effects:
Formula: ~1|ARSBFGLNGS102860

| | | |
|---|---|---|
| | (Intercept) | Residual |
| StdDev: | 0.1681112 | 1.533913 |

Fixed effects: list(fixed)

| | | | | | |
|---|---|---|---|---|---|
| | Value | Std. Error | DF | t-value | p-value |
| (Intercept) | 2.6991009 | 0.1154812 | 1449 | 23.372651 | 0.0000 |
| HAPMAP53129RS29022984G/A | −0.3145608 | 0.0985850 | 1449 | −3.190757 | 0.0014 |
| HAPMAP53129RS29022984A/A | −0.1747556 | 0.2671308 | 1449 | −0.654195 | 0.5131 |
| Correlation: | | | | | |

| | | |
|---|---|---|
| | (Intr) | HAPMAP53129RS29022984G |
| HAPMAP53129RS29022984G/A | −0.179 | |
| HAPMAP53129RS29022984A/A | −0.067 | 0.080 |

Standardized Within-Group Residuals:

| | | | | |
|---|---|---|---|---|
| Min | Q1 | Med | Q3 | Max |
| −1.7732710 | −0.7382653 | −0.0903765 | 0.6307824 | 4.8031184 |

Number of Observations: 1454
Number of Groups: 3

| | | | | |
|---|---|---|---|---|
| | numDF | denDF | F-value | p-value |
| (Intercept) | 1 | 1449 | 537.6894 | <.0001 |
| HAPMAP53129RS29022984 | 2 | 1449 | 5.1705 | 0.0058 |

HAPMAP53129RS29022984 × BFGLNGS119018
Linear mixed-effects model fit by REML
Data: vm

| | | |
|---|---|---|
| AIC | BIC | logLik |
| 5389.17 | 5415.57 | −2689.585 |

Random effects:
Formula: ~1|BFGLNGS119018

| | | |
|---|---|---|
| | (Intercept) | Residual |
| StdDev: | 0.03127218 | 1.535703 |

Fixed effects: list(fixed)

| | | | | | |
|---|---|---|---|---|---|
| | Value | Std. Error | DF | t-value | p-value |
| (Intercept) | 2.747316 | 0.05229185 | 1449 | 52.53814 | 0.0000 |
| HAPMAP53129RS29022984G/A | −0.311417 | 0.09898761 | 1449 | −3.14602 | 0.0017 |
| HAPMAP53129RS29022984A/A | −0.158476 | 0.26776224 | 1449 | −0.59185 | 0.5540 |
| Correlation: | | | | | |

| | | |
|---|---|---|
| | (Intr) | HAPMAP53129RS29022984G |
| HAPMAP53129RS29022984G/A | −0.430 | |
| HAPMAP53129RS29022984A/A | −0.167 | 0.084 |

Standardized Within-Group Residuals:

| | | | | |
|---|---|---|---|---|
| Min | Q1 | Med | Q3 | Max |
| −1.7304527 | −0.7537013 | −0.1025336 | 0.6137507 | 4.7812236 |

Number of Observations: 1454
Number of Groups: 3

| | | | | |
|---|---|---|---|---|
| | numDF | denDF | F-value | p-value |
| (Intercept) | 1 | 1449 | 3278.044 | <.0001 |
| HAPMAP53129RS29022984 | 2 | 1449 | 5.002 | 0.0068 |

HAPMAP53129RS29022984 × ARSBFGLBAC20850
Linear mixed-effects model fit by REML
Data: vm

| | | |
|---|---|---|
| AIC | BIC | logLik |
| 5389.193 | 5415.593 | −2689.597 |

Random effects:
Formula: ~1|ARSBFGLBAC20850

| | | |
|---|---|---|
| | (Intercept) | Residual |
| StdDev: | 9.493588e−05 | 1.535827 |

Fixed effects: list(fixed)

| | | | | | |
|---|---|---|---|---|---|
| | Value | Std. Error | DF | t-value | p-value |
| (Intercept) | 2.7534234 | 0.04609794 | 1449 | 59.72986 | 0.0000 |
| HAPMAP53129RS29022984G/A | −0.3153589 | 0.09866070 | 1449 | −3.19640 | 0.0014 |
| HAPMAP53129RS29022984A/A | −0.1651881 | 0.26739569 | 1449 | −0.61777 | 0.5368 |
| Correlation: | | | | | |

| | | |
|---|---|---|
| | (Intr) | HAPMAP53129RS29022984G |
| HAPMAP53129RS29022984G/A | −0.467 | |
| HAPMAP53129RS29022984A/A | −0.172 | 0.081 |

Standardized Within-Group Residuals:

| | | | | |
|---|---|---|---|---|
| Min | Q1 | Med | Q3 | Max |
| −1.72768361 | −0.75101122 | −0.09989628 | 0.61633014 | 4.78346571 |

Number of Observations: 1454
Number of Groups: 3

| | | | | |
|---|---|---|---|---|
| | numDF | denDF | F-value | p-value |
| (Intercept) | 1 | 1449 | 4435.071 | <.0001 |
| HAPMAP53129RS29022984 | 2 | 1449 | 5.174 | 0.0058 |

HAPMAP53129RS29022984 × ARSBFGLNGS100843
Linear mixed-effects model fit by REML
Data: vm

| | | |
|---|---|---|
| AIC | BIC | logLik |
| 5387.808 | 5414.208 | −2688.904 |

Random effects:
Formula: ~1|ARSBFGLNGS100843

| | | |
|---|---|---|
| | (Intercept) | Residual |
| StdDev: | 0.1381296 | 1.534302 |

Fixed effects: list(fixed)

| | | | | | |
|---|---|---|---|---|---|
| | Value | Std. Error | DF | t-value | p-value |
| (Intercept) | 2.8354538 | 0.11073206 | 1449 | 25.606441 | 0.0000 |
| HAPMAP53129RS29022984G/A | −0.3191891 | 0.09859123 | 1449 | −3.237500 | 0.0012 |
| HAPMAP53129RS29022984A/A | −0.1511138 | 0.26726308 | 1449 | −0.565412 | 0.5719 |
| Correlation: | | | | | |

| | | |
|---|---|---|
| | (Intr) | HAPMAP53129RS29022984G |
| HAPMAP53129RS29022984G/A | −0.207 | |
| HAPMAP53129RS29022984A/A | −0.060 | 0.080 |

Standardized Within-Group Residuals:

| | | | | |
|---|---|---|---|---|
| Min | Q1 | Med | Q3 | Max |
| −1.8185024 | −0.7364424 | −0.1239208 | 0.6322581 | 4.8035358 |

Number of Observations: 1454
Number of Groups: 3

| | | | | |
|---|---|---|---|---|
| | numDF | denDF | F-value | p-value |
| (Intercept) | 1 | 1449 | 650.2304 | <.0001 |
| HAPMAP53129RS29022984 | 2 | 1449 | 5.2881 | 0.0051 |

HAPMAP53129RS29022984 × ARSBFGLNGS97162
Linear mixed-effects model fit by REML
Data: vm

| | | |
|---|---|---|
| AIC | BIC | logLik |
| 5385.115 | 5411.515 | −2687.558 |

Random effects:
Formula: ~1|ARSBFGLNGS97162

| | | |
|---|---|---|
| | (Intercept) | Residual |
| StdDev: | 0.1589232 | 1.532557 |

Fixed effects: list(fixed)

| | | | | | |
|---|---|---|---|---|---|
| | Value | Std. Error | DF | t-value | p-value |
| (Intercept) | 2.8212596 | 0.11996648 | 1449 | 23.517065 | 0.0000 |
| HAPMAP53129RS29022984G/A | −0.3084629 | 0.09849319 | 1449 | −3.131819 | 0.0018 |
| HAPMAP53129RS29022984A/A | −0.1656440 | 0.26682999 | 1449 | −0.620785 | 0.5348 |
| Correlation: | | | | | |

| | | |
|---|---|---|
| | (Intr) | HAPMAP53129RS29022984G |
| HAPMAP53129RS29022984G/A | −0.169 | |
| HAPMAP53129RS29022984A/A | −0.064 | 0.081 |

Standardized Within-Group Residuals:

| | | | | |
|---|---|---|---|---|
| Min | Q1 | Med | Q3 | Max |
| −1.8255905 | −0.7163334 | −0.1265226 | 0.5967540 | 4.8325091 |

Number of Observations: 1454
Number of Groups: 3

| | | | | |
|---|---|---|---|---|
| | numDF | denDF | F-value | p-value |
| (Intercept) | 1 | 1449 | 544.5067 | <.0001 |
| HAPMAP53129RS29022984 | 2 | 1449 | 4.9725 | 0.007 |

HAPMAP53129RS29022984 × Hapmap42294BTA69421
Linear mixed-effects model fit by REML
Data: vm

| | | |
|---|---|---|
| AIC | BIC | logLik |
| 5388.28 | 5414.68 | −2689.14 |

Random effects:
Formula: ~1|Hapmap42294BTA69421

| | | |
|---|---|---|
| | (Intercept) | Residual |
| StdDev: | 0.085083 | 1.534439 |

Fixed effects: list(fixed)

| | | | | | |
|---|---|---|---|---|---|
| | Value | Std. Error | DF | t-value | p-value |
| (Intercept) | 2.7482431 | 0.06838687 | 1449 | 40.18671 | 0.0000 |
| HAPMAP53129RS29022984G/A | −0.3167603 | 0.09858182 | 1449 | −3.21317 | 0.0013 |
| HAPMAP53129RS29022984A/A | −0.1702677 | 0.26717431 | 1449 | −0.63729 | 0.5240 |
| Correlation: | | | | | |

| | | |
|---|---|---|
| | (Intr) | HAPMAP53129RS29022984G |
| HAPMAP53129RS29022984G/A | −0.312 | |
| HAPMAP53129RS29022984A/A | −0.115 | 0.081 |

Standardized Within-Group Residuals:

| | | | | |
|---|---|---|---|---|
| Min | Q1 | Med | Q3 | Max |
| −1.7727337 | −0.7348995 | −0.1283331 | 0.6251252 | 4.7443063 |

Number of Observations: 1454
Number of Groups: 3

| | | | | |
|---|---|---|---|---|
| | numDF | denDF | F-value | p-value |
| (Intercept) | 1 | 1449 | 1713.6622 | <.0001 |
| HAPMAP53129RS29022984 | 2 | 1449 | 5.2342 | 0.0054 |

HAPMAP53129RS29022984 × ARSBFGLBAC2384
Linear mixed-effects model fit by REML
Data: vm

| | | |
|---|---|---|
| AIC | BIC | logLik |
| 5387.789 | 5414.189 | −2688.895 |

Random effects:
Formula: ~1|ARSBFGLBAC2384

| | | |
|---|---|---|
| | (Intercept) | Residual |
| StdDev: | 0.08429043 | 1.534285 |

Fixed effects: list(fixed)

| | | | | | |
|---|---|---|---|---|---|
| | Value | Std. Error | DF | t-value | p-value |
| (Intercept) | 2.7514684 | 0.07017441 | 1449 | 39.20900 | 0.0000 |
| HAPMAP53129RS29022984G/A | −0.3111823 | 0.09859226 | 1449 | −3.15626 | 0.0016 |
| HAPMAP53129RS29022984A/A | −0.1673459 | 0.26713050 | 1449 | −0.62646 | 0.5311 |
| Correlation: | | | | | |

| | | |
|---|---|---|
| | (Intr) | HAPMAP53129RS29022984G |
| HAPMAP53129RS29022984G/A | −0.306 | |
| HAPMAP53129RS29022984A/A | −0.114 | 0.080 |

Standardized Within-Group Residuals:

| | | | | |
|---|---|---|---|---|
| Min | Q1 | Med | Q3 | Max |
| −1.7712437 | −0.7398752 | −0.1311278 | 0.6340161 | 4.7464505 |

Number of Observations: 1454
Number of Groups: 3

| | | | | |
|---|---|---|---|---|
| | numDF | denDF | F-value | p-value |
| (Intercept) | 1 | 1449 | 1625.2314 | <.0001 |
| HAPMAP53129RS29022984 | 2 | 1449 | 5.0509 | 0.0065 |

HAPMAP53129RS29022984 × BTB01553536
Linear mixed-effects model fit by REML
Data: vm

| | | |
|---|---|---|
| AIC | BIC | logLik |
| 5387.451 | 5413.851 | −2688.725 |

Random effects:
Formula: ~1|BTB01553536

| | | |
|---|---|---|
| | (Intercept) | Residual |
| StdDev: | 0.08966774 | 1.533916 |

Fixed effects: list(fixed)

| | | | | | |
|---|---|---|---|---|---|
| | Value | Std. Error | DF | t-value | p-value |
| (Intercept) | 2.7534848 | 0.06998392 | 1449 | 39.34454 | 0.0000 |
| HAPMAP53129RS29022984G/A | −0.3135617 | 0.09858010 | 1449 | −3.18078 | 0.0015 |
| HAPMAP53129RS29022984A/A | −0.1674178 | 0.26706587 | 1449 | −0.62688 | 0.5308 |
| Correlation: | | | | | |

| | | |
|---|---|---|
| | (Intr) | HAPMAP53129RS29022984G |
| HAPMAP53129RS29022984G/A | −0.303 | |
| HAPMAP53129RS29022984A/A | −0.114 | 0.080 |

Standardized Within-Group Residuals:

| | | | | |
|---|---|---|---|---|
| Min | Q1 | Med | Q3 | Max |
| −1.7754432 | −0.7515712 | −0.1201015 | 0.6174734 | 4.7438167 |

Number of Observations: 1454
Number of Groups: 3

| | | | | |
|---|---|---|---|---|
| | numDF | denDF | F-value | p-value |
| (Intercept) | 1 | 1449 | 1633.875 | <.0001 |
| HAPMAP53129RS29022984 | 2 | 1449 | 5.128 | 0.006 |

HAPMAP53129RS29022984 × HAPMAP53129RS29022984
Linear mixed-effects model fit by REML
Data: vm

| | | |
|---|---|---|
| AIC | BIC | logLik |
| 5389.193 | 5415.593 | −2689.597 |

Random effects:
Formula: ~1|HAPMAP53129RS29022984

| | | |
|---|---|---|
| | (Intercept) | Residual |
| StdDev: | 0.1860328 | 1.535827 |

Fixed effects: list(fixed)

| | | | | | |
|---|---|---|---|---|---|
| | Value | Std. Error | DF | t-value | p-value |
| (Intercept) | 2.7534234 | 0.1916591 | 1451 | 14.366254 | 0 |
| HAPMAP53129RS29022984G/A | −0.3153589 | 0.2809810 | 0 | −1.122350 | NaN |
| HAPMAP53129RS29022984A/A | −0.1651881 | 0.3751225 | 0 | −0.440358 | NaN |
| Correlation: | | | | | |

| | | |
|---|---|---|
| | (Intr) | HAPMAP53129RS29022984G |
| HAPMAP53129RS29022984G/A | −0.682 | |
| HAPMAP53129RS29022984A/A | −0.511 | 0.349 |

Standardized Within-Group Residuals:

| | | | | |
|---|---|---|---|---|
| Min | Q1 | Med | Q3 | Max |
| −1.72768361 | −0.75101121 | −0.09989628 | 0.61633014 | 4.78346571 |

Number of Observations: 1454
Number of Groups: 3

| | | | | |
|---|---|---|---|---|
| | numDF | denDF | F-value | p-value |
| (Intercept) | 1 | 1451 | 410.3498 | <.0001 |
| HAPMAP53129RS29022984 | 2 | 0 | 0.6312 | NaN |

HAPMAP53129RS29022984 × ARSBFGLNGS68110
Linear mixed-effects model fit by REML
Data: vm

| | | |
|---|---|---|
| AIC | BIC | logLik |
| 5356.992 | 5383.393 | −2673.496 |

Random effects:
Formula: ~1|ARSBFGLNGS68110

| | | |
|---|---|---|
| | (Intercept) | Residual |
| StdDev: | 0.3005834 | 1.515885 |

Fixed effects: list(fixed)

| | | | | | |
|---|---|---|---|---|---|
| | Value | Std. Error | DF | t-value | p-value |
| (Intercept) | 2.6955505 | 0.18039538 | 1449 | 14.942458 | 0.0000 |
| HAPMAP53129RS29022984G/A | −0.2292425 | 0.09839595 | 1449 | −2.329796 | 0.0200 |
| HAPMAP53129RS29022984A/A | −0.0009033 | 0.26548584 | 1449 | −0.003403 | 0.9973 |
| Correlation: | | | | | |

| | | |
|---|---|---|
| | (Intr) | HAPMAP53129RS29022984G |
| HAPMAP53129RS29022984G/A | −0.122 | |
| HAPMAP53129RS29022984A/A | −0.052 | 0.092 |

Standardized Within-Group Residuals:

| | | | | |
|---|---|---|---|---|
| Min | Q1 | Med | Q3 | Max |
| −1.93397483 | −0.72438226 | −0.09283546 | 0.60073266 | 4.66283005 |

Number of Observations: 1454
Number of Groups: 3

| | | | | |
|---|---|---|---|---|
| | numDF | denDF | F-value | p-value |
| (Intercept) | 1 | 1449 | 218.75127 | <.0001 |
| HAPMAP53129RS29022984 | 2 | 1449 | 2.73656 | 0.0651 |

HAPMAP53129RS29022984 × HAPMAP49592BTA38891
Linear mixed-effects model fit by REML
Data: vm

| | | |
|---|---|---|
| AIC | BIC | logLik |
| 5389.18 | 5415.58 | −2689.59 |

Random effects:
Formula: ~1|HAPMAP49592BTA38891

| | | |
|---|---|---|
| | (Intercept) | Residual |
| StdDev: | 0.03223680 | 1.535686 |

| | | | | | |
|---|---|---|---|---|---|
| Fixed effects: list(fixed) | | | | | |
| | Value | Std. Error | DF | t-value | p-value |
| (Intercept) | 2.7533684 | 0.05176668 | 1449 | 53.18804 | 0.0000 |
| HAPMAP53129RS29022984G/A | −0.3148074 | 0.09865889 | 1449 | −3.19087 | 0.0014 |
| HAPMAP53129RS29022984A/A | −0.1658836 | 0.26737643 | 1449 | −0.62041 | 0.5351 |

| | | |
|---|---|---|
| Correlation: | | |
| | (Intr) | HAPMAP53129RS29022984G |
| HAPMAP53129RS29022984G/A | −0.414 | |
| HAPMAP53129RS29022984A/A | −0.155 | 0.080 |

Standardized Within-Group Residuals:

| | | | | |
|---|---|---|---|---|
| Min | Q1 | Med | Q3 | Max |
| −1.7359385 | −0.7479633 | −0.1031804 | 0.6195038 | 4.7870224 |

Number of Observations: 1454
Number of Groups: 3

| | | | | |
|---|---|---|---|---|
| | numDF | denDF | F-value | p-value |
| (Intercept) | 1 | 1449 | 3301.433 | <.0001 |
| HAPMAP53129RS29022984 | 2 | 1449 | 5.157 | 0.0059 |

HAPMAP53129RS29022984 × ARSBFGLNGS30157
Linear mixed-effects model fit by REML
Data: vm

| | | |
|---|---|---|
| AIC | BIC | logLik |
| 5385.236 | 5411.636 | −2687.618 |

Random effects:
Formula: ~1|ARSBFGLNGS30157

| | | |
|---|---|---|
| | (Intercept) | Residual |
| StdDev: | 0.1838738 | 1.532531 |

| | | | | | |
|---|---|---|---|---|---|
| Fixed effects: list(fixed) | | | | | |
| | Value | Std. Error | DF | t-value | p-value |
| (Intercept) | 2.8557923 | 0.13590322 | 1449 | 21.013427 | 0.0000 |
| HAPMAP53129RS29022984G/A | −0.3110236 | 0.09846989 | 1449 | −3.158566 | 0.0016 |
| HAPMAP53129RS29022984A/A | −0.1799198 | 0.26690490 | 1449 | −0.674097 | 0.5004 |

| | | |
|---|---|---|
| Correlation: | | |
| | (Intr) | HAPMAP53129RS29022984G |
| HAPMAP53129RS29022984G/A | −0.148 | |
| HAPMAP53129RS29022984A/A | −0.070 | 0.080 |

Standardized Within-Group Residuals:

| | | | | |
|---|---|---|---|---|
| Min | Q1 | Med | Q3 | Max |
| −1.8413928 | −0.7249235 | −0.1254084 | 0.5923585 | 4.8265158 |

Number of Observations: 1454
Number of Groups: 3

| | | | | |
|---|---|---|---|---|
| | numDF | denDF | F-value | p-value |
| (Intercept) | 1 | 1449 | 432.0566 | <.0001 |
| HAPMAP53129RS29022984 | 2 | 1449 | 5.0776 | 0.0063 |

HAPMAP53129RS29022984 × HAPMAP30097BTC007678
Linear mixed-effects model fit by REML
Data: vm

| | | |
|---|---|---|
| AIC | BIC | logLik |
| 5389.193 | 5415.593 | −2689.597 |

Random effects:
Formula: ~1|HAPMAP30097BTC007678

| | | |
|---|---|---|
| | (Intercept) | Residual |
| StdDev: | 0.0001152174 | 1.535827 |

| | | | | | |
|---|---|---|---|---|---|
| Fixed effects: list(fixed) | | | | | |
| | Value | Std. Error | DF | t-value | p-value |
| (Intercept) | 2.7534235 | 0.04609797 | 1449 | 59.72982 | 0.0000 |
| HAPMAP53129RS29022984G/A | −0.3153589 | 0.09866070 | 1449 | −3.19640 | 0.0014 |
| HAPMAP53129RS29022984A/A | −0.1651881 | 0.26739569 | 1449 | −0.61777 | 0.5368 |

| | | |
|---|---|---|
| Correlation: | | |
| | (Intr) | HAPMAP53129RS29022984G |
| HAPMAP53129RS29022984G/A | −0.467 | |
| HAPMAP53129RS29022984A/A | −0.172 | 0.081 |

Standardized Within-Group Residuals:

| | | | | |
|---|---|---|---|---|
| Min | Q1 | Med | Q3 | Max |
| −1.72768369 | −0.75101120 | −0.09989627 | 0.61633016 | 4.78346573 |

Number of Observations: 1454
Number of Groups: 3

| | | | | |
|---|---|---|---|---|
| | numDF | denDF | F-value | p-value |
| (Intercept) | 1 | 1449 | 4435.064 | <.0001 |
| HAPMAP53129RS29022984 | 2 | 1449 | 5.174 | 0.0058 |

ARSBFGLNGS68110 × ARSBFGLNGS102860
Linear mixed-effects model fit by REML
Data: vm

| | | |
|---|---|---|
| AIC | BIC | logLik |
| 5355.787 | 5382.187 | −2672.893 |

Random effects:
Formula: ~1|ARSBFGLNGS102860

| | | |
|---|---|---|
| | (Intercept) | Residual |
| StdDev: | 0.1258409 | 1.516069 |

| | | | | | |
|---|---|---|---|---|---|
| Fixed effects: list(fixed) | | | | | |
| | Value | Std. Error | DF | t-value | p-value |
| (Intercept) | 2.9858616 | 0.10353041 | 1449 | 28.840431 | 0 |
| ARSBFGLNGS68110C/A | −0.5401614 | 0.08708108 | 1449 | −6.202971 | 0 |
| ARSBFGLNGS68110A/A | −0.5738831 | 0.11693625 | 1449 | −4.907658 | 0 |

| | | |
|---|---|---|
| Correlation: | | |
| | (Intr) | ARSBFGLNGS68110C |
| ARSBFGLNGS68110C/A | −0.448 | |
| ARSBFGLNGS68110A/A | −0.345 | 0.392 |

Standardized Within-Group Residuals:

| | | | | |
|---|---|---|---|---|
| Min | Q1 | Med | Q3 | Max |
| −1.96778736 | −0.71583003 | −0.09569915 | 0.60337089 | 4.69289376 |

Number of Observations: 1454
Number of Groups: 3

| | | | | |
|---|---|---|---|---|
| | numDF | denDF | F-value | p-value |
| (Intercept) | 1 | 1449 | 853.9731 | <.0001 |
| ARSBFGLNGS68110 | 2 | 1449 | 22.8588 | <.0001 |

ARSBFGLNGS68110 × BFGLNGS119018
Linear mixed-effects model fit by REML
Data: vm

| | | |
|---|---|---|
| AIC | BIC | logLik |
| 5356.577 | 5382.977 | −2673.288 |

Random effects:
Formula: ~1|BFGLNGS119018

| | | |
|---|---|---|
| | (Intercept) | Residual |
| StdDev: | 7.786096e−05 | 1.517608 |

| | | | | | |
|---|---|---|---|---|---|
| Fixed effects: list(fixed) | | | | | |
| | Value | Std. Error | DF | t-value | p-value |
| (Intercept) | 3.0135182 | 0.06317889 | 1449 | 47.69818 | 0 |
| ARSBFGLNGS68110C/A | −0.5399658 | 0.08715416 | 1449 | −6.19552 | 0 |
| ARSBFGLNGS68110A/A | −0.5736022 | 0.11691281 | 1449 | −4.90624 | 0 |

| | | |
|---|---|---|
| Correlation: | | |
| | (Intr) | ARSBFGLNGS68110C |
| ARSBFGLNGS68110C/A | −0.725 | |
| ARSBFGLNGS68110A/A | −0.540 | 0.392 |

Standardized Within-Group Residuals:

| | | | | |
|---|---|---|---|---|
| Min | Q1 | Med | Q3 | Max |
| −1.9198092 | −0.7337323 | −0.1032771 | 0.6104656 | 4.6695068 |

Number of Observations: 1454
Number of Groups: 3

| | | | | |
|---|---|---|---|---|
| | numDF | denDF | F-value | p-value |
| (Intercept) | 1 | 1449 | 4542.205 | <.0001 |
| ARSBFGLNGS68110 | 2 | 1449 | 22.823 | <.0001 |

ARSBFGLNGS68110 × ARSBFGLBAC20850
Linear mixed-effects model fit by REML
Data: vm

| | | |
|---|---|---|
| AIC | BIC | logLik |
| 5356.577 | 5382.977 | −2673.288 |

Random effects:
Formula: ~1|ARSBFGLBAC20850

| | | |
|---|---|---|
| | (Intercept) | Residual |
| StdDev: | 0.0001174213 | 1.517608 |

| | | | | | |
|---|---|---|---|---|---|
| Fixed effects: list(fixed) | | | | | |
| | Value | Std. Error | DF | t-value | p-value |
| (Intercept) | 3.0135182 | 0.06317893 | 1449 | 47.69815 | 0 |
| ARSBFGLNGS68110C/A | −0.5399658 | 0.08715416 | 1449 | −6.19553 | 0 |
| ARSBFGLNGS68110A/A | −0.5736022 | 0.11691281 | 1449 | −4.90624 | 0 |

| | | |
|---|---|---|
| Correlation: | | |
| | (Intr) | ARSBFGLNGS68110C |
| ARSBFGLNGS68110C/A | −0.725 | |
| ARSBFGLNGS68110A/A | −0.540 | 0.392 |

Standardized Within-Group Residuals:

| | | | | |
|---|---|---|---|---|
| Min | Q1 | Med | Q3 | Max |
| −1.9198092 | −0.7337323 | −0.1032771 | 0.6104656 | 4.6695068 |

Number of Observations: 1454
Number of Groups: 3

| | | | | |
|---|---|---|---|---|
| | numDF | denDF | F-value | p-value |
| (Intercept) | 1 | 1449 | 4542.186 | <.0001 |
| ARSBFGLNGS68110 | 2 | 1449 | 22.823 | <.0001 |

ARSBFGLNGS68110 × ARSBFGLNGS100843
Linear mixed-effects model fit by REML
Data: vm

| | | |
|---|---|---|
| AIC | BIC | logLik |
| 5354.604 | 5381.004 | −2672.302 |

Random effects:
Formula: ~1|ARSBFGLNGS100843

| | | |
|---|---|---|
| | (Intercept) | Residual |
| StdDev: | 0.1514786 | 1.515695 |

| | | | | | |
|---|---|---|---|---|---|
| Fixed effects: list(fixed) | | | | | |
| | Value | Std. Error | DF | t-value | p-value |
| (Intercept) | 3.1084278 | 0.12615467 | 1449 | 24.639815 | 0 |
| ARSBFGLNGS68110C/A | −0.5421602 | 0.08705211 | 1449 | −6.227996 | 0 |
| ARSBFGLNGS68110A/A | −0.5843324 | 0.11691255 | 1449 | −4.998030 | 0 |

| | | |
|---|---|---|
| Correlation: | | |
| | (Intr) | ARSBFGLNGS68110C |
| ARSBFGLNGS68110C/A | −0.367 | |
| ARSBFGLNGS68110A/A | −0.284 | 0.392 |

Standardized Within-Group Residuals:

| | | | | |
|---|---|---|---|---|
| Min | Q1 | Med | Q3 | Max |
| −1.9616723 | −0.7285831 | −0.0966436 | 0.6012802 | 4.6918123 |

Number of Observations: 1454
Number of Groups: 3

| | | | | |
|---|---|---|---|---|
| | numDF | denDF | F-value | p-value |
| (Intercept) | 1 | 1449 | 571.1068 | <.0001 |
| ARSBFGLNGS68110 | 2 | 1449 | 23.2575 | <.0001 |

ARSBFGLNGS68110 × ARSBFGLNGS97162
Linear mixed-effects model fit by REML
Data: vm

| | | |
|---|---|---|
| AIC | BIC | logLik |
| 5355.092 | 5381.493 | −2672.546 |

Random effects:
Formula: ~1|ARSBFGLNGS97162

| | | |
|---|---|---|
| | (Intercept) | Residual |
| StdDev: | 0.1100114 | 1.516071 |

| | | | | | |
|---|---|---|---|---|---|
| Fixed effects: list(fixed) | | | | | |
| | Value | Std. Error | DF | t-value | p-value |
| (Intercept) | 3.0478225 | 0.09991199 | 1449 | 30.505072 | 0 |
| ARSBFGLNGS68110C/A | −0.5259664 | 0.08746377 | 1449 | −6.013534 | 0 |
| ARSBFGLNGS68110A/A | −0.5570883 | 0.11721708 | 1449 | −4.752620 | 0 |

| | | |
|---|---|---|
| Correlation: | | |
| | (Intr) | ARSBFGLNGS68110C |
| ARSBFGLNGS68110C/A | −0.445 | |
| ARSBFGLNGS68110A/A | −0.332 | 0.397 |

Standardized Within-Group Residuals:

| | | | | |
|---|---|---|---|---|
| Min | Q1 | Med | Q3 | Max |
| −1.8915319 | −0.7325571 | −0.0934855 | 0.6143001 | 4.7044644 |

Number of Observations: 1454
Number of Groups: 3

| | | | | |
|---|---|---|---|---|
| | numDF | denDF | F-value | p-value |
| (Intercept) | 1 | 1449 | 970.2681 | <.0001 |
| ARSBFGLNGS68110 | 2 | 1449 | 21.4071 | <.0001 |

ARSBFGLNGS68110 × Hapmap42294BTA69421
Linear mixed-effects model fit by REML
Data: vm

| | | |
|---|---|---|
| AIC | BIC | logLik |
| 5354.654 | 5381.054 | −2672.327 |

Random effects:
Formula: ~1|Hapmap42294BTA69421

| | | |
|---|---|---|
| | (Intercept) | Residual |
| StdDev: | 0.1109109 | 1.515343 |

| | | | | | |
|---|---|---|---|---|---|
| Fixed effects: list(fixed) | | | | | |
| | Value | Std. Error | DF | t-value | p-value |
| (Intercept) | 3.0073171 | 0.09064648 | 1449 | 33.17633 | 0 |
| ARSBFGLNGS68110C/A | −0.5474228 | 0.08711962 | 1449 | −6.28358 | 0 |
| ARSBFGLNGS68110A/A | −0.5819597 | 0.11682795 | 1449 | −4.98134 | 0 |

| | | |
|---|---|---|
| Correlation: | | |
| | (Intr) | ARSBFGLNGS68110C |
| ARSBFGLNGS68110C/A | −0.500 | |
| ARSBFGLNGS68110A/A | −0.372 | 0.393 |

Standardized Within-Group Residuals:

| | | | | |
|---|---|---|---|---|
| Min | Q1 | Med | Q3 | Max |
| −1.98472303 | −0.73981037 | −0.09328473 | 0.62952929 | 4.61444266 |

Number of Observations: 1454
Number of Groups: 3

| | | | | |
|---|---|---|---|---|
| | numDF | denDF | F-value | p-value |
| (Intercept) | 1 | 1449 | 1220.6070 | <.0001 |
| ARSBFGLNGS68110 | 2 | 1449 | 23.4752 | <.0001 |

ARSBFGLNGS68110 × ARSBFGLBAC2384
Linear mixed-effects model fit by REML
Data: vm

| | | |
|---|---|---|
| AIC | BIC | logLik |
| 5356.577 | 5382.977 | −2673.288 |

Random effects:
Formula: ~1|ARSBFGLBAC2384

| | | |
|---|---|---|
| | (Intercept) | Residual |
| StdDev: | 0.0003074777 | 1.517608 |

| | | | | | |
|---|---|---|---|---|---|
| Fixed effects: list(fixed) | | | | | |
| | Value | Std. Error | DF | t-value | p-value |
| (Intercept) | 3.0135180 | 0.06317919 | 1449 | 47.69795 | 0 |
| ARSBFGLNGS68110C/A | −0.5399654 | 0.08715418 | 1449 | −6.19552 | 0 |
| ARSBFGLNGS68110A/A | −0.5736017 | 0.11691284 | 1449 | −4.90623 | 0 |

| | | |
|---|---|---|
| Correlation: | | |
| | (Intr) | ARSBFGLNGS68110C |
| ARSBFGLNGS68110C/A | −0.725 | |
| ARSBFGLNGS68110A/A | −0.540 | 0.392 |

Standardized Within-Group Residuals:

| | | | | |
|---|---|---|---|---|
| Min | Q1 | Med | Q3 | Max |
| −1.9198100 | −0.7337314 | −0.1032773 | 0.6104646 | 4.6695061 |

Number of Observations: 1454
Number of Groups: 3

| | | | | |
|---|---|---|---|---|
| | numDF | denDF | F-value | p-value |
| (Intercept) | 1 | 1449 | 4542.100 | <.0001 |
| ARSBFGLNGS68110 | 2 | 1449 | 22.823 | <.0001 |

ARSBFGLNGS68110 × BTB01553536
Linear mixed-effects model fit by REML
Data: vm

| | | |
|---|---|---|
| AIC | BIC | logLik |
| 5355.497 | 5381.897 | −2672.748 |

Random effects:
Formula: ~1|BTB01553536

| | | |
|---|---|---|
| | (Intercept) | Residual |
| StdDev: | 0.07615653 | 1.516246 |

| | | | | | |
|---|---|---|---|---|---|
| Fixed effects: list(fixed) | | | | | |
| | Value | Std. Error | DF | t-value | p-value |
| (Intercept) | 3.0131589 | 0.07765148 | 1449 | 38.80362 | 0 |
| ARSBFGLNGS68110C/A | −0.5365654 | 0.08711688 | 1449 | −6.15914 | 0 |
| ARSBFGLNGS68110A/A | −0.5667224 | 0.11690800 | 1449 | −4.84759 | 0 |

| | | |
|---|---|---|
| Correlation: | | |
| | (Intr) | ARSBFGLNGS68110C |
| ARSBFGLNGS68110C/A | −0.592 | |
| ARSBFGLNGS68110A/A | −0.442 | 0.392 |

Standardized Within-Group Residuals:

| | | | | |
|---|---|---|---|---|
| Min | Q1 | Med | Q3 | Max |
| −1.9545821 | −0.7170216 | −0.1079162 | 0.5813766 | 4.6406533 |

Number of Observations: 1454
Number of Groups: 3

| | | | | |
|---|---|---|---|---|
| | numDF | denDF | F-value | p-value |
| (Intercept) | 1 | 1449 | 1996.7485 | <.0001 |
| ARSBFGLNGS68110 | 2 | 1449 | 22.4585 | <.0001 |

ARSBFGLNGS68110 × HAPMAP53129RS29022984
Linear mixed-effects model fit by REML
Data: vm

| | | |
|---|---|---|
| AIC | BIC | logLik |
| 5354.388 | 5380.788 | −2672.194 |

Random effects:
Formula: ~1|HAPMAP53129RS29022984

| | | |
|---|---|---|
| | (Intercept) | Residual |
| StdDev: | 0.1203776 | 1.515597 |

| | | | | | |
|---|---|---|---|---|---|
| Fixed effects: list(fixed) | | | | | |
| | Value | Std. Error | DF | t-value | p-value |
| (Intercept) | 2.9630370 | 0.10668040 | 1449 | 27.774895 | 0 |
| ARSBFGLNGS68110C/A | −0.5183818 | 0.08779659 | 1449 | −5.904350 | 0 |
| ARSBFGLNGS68110A/A | −0.5551499 | 0.11729876 | 1449 | −4.732786 | 0 |

| | | |
|---|---|---|
| Correlation: | | |
| | (Intr) | ARSBFGLNGS68110C |
| ARSBFGLNGS68110C/A | −0.471 | |
| ARSBFGLNGS68110A/A | −0.359 | 0.399 |

Standardized Within-Group Residuals:

| | | | | |
|---|---|---|---|---|
| Min | Q1 | Med | Q3 | Max |
| −1.9384314 | −0.7125091 | −0.0909740 | 0.6052205 | 4.6596307 |

Number of Observations: 1454
Number of Groups: 3

| | | | | |
|---|---|---|---|---|
| | numDF | denDF | F-value | p-value |
| (Intercept) | 1 | 1449 | 808.2711 | <.0001 |
| ARSBFGLNGS68110 | 2 | 1449 | 20.7953 | <.0001 |

ARSBFGLNGS68110 × ARSBFGLNGS68110
Linear mixed-effects model fit by REML
Data: vm

| | | |
|---|---|---|
| AIC | BIC | logLik |
| 5356.577 | 5382.977 | −2673.288 |

Random effects:
Formula: ~1|ARSBFGLNGS68110

| | | |
|---|---|---|
| | (Intercept) | Residual |
| StdDev: | 0.1838260 | 1.517608 |

| | | | | | |
|---|---|---|---|---|---|
| Fixed effects: list(fixed) | | | | | |
| | Value | Std. Error | DF | t-value | p-value |
| (Intercept) | 3.0135182 | 0.1943799 | 1451 | 15.503239 | 0 |
| ARSBFGLNGS68110C/A | −0.5399658 | 0.2741894 | 0 | −1.969317 | NaN |
| ARSBFGLNGS68110A/A | −0.5736022 | 0.2850484 | 0 | −2.012298 | NaN |

| | | |
|---|---|---|
| Correlation: | | |
| | (Intr) | ARSBFGLNGS68110C |
| ARSBFGLNGS68110C/A | −0.709 | |
| ARSBFGLNGS68110A/A | −0.682 | 0.483 |

Standardized Within-Group Residuals:

| | | | | |
|---|---|---|---|---|
| Min | Q1 | Med | Q3 | Max |
| −1.9198092 | −0.7337323 | −0.1032771 | 0.6104656 | 4.6695068 |

Number of Observations: 1454
Number of Groups: 3

| | | | | |
|---|---|---|---|---|
| | numDf | denDF | F-value | p-value |
| (Intercept) | 1 | 1451 | 535.5693 | <.0001 |
| ARSBFGLNGS68110 | 2 | 0 | 2.6726 | NaN |

ARSBFGLNGS68110 × HAPMAP49592BTA38891
Linear mixed-effects model fit by REML
Data: vm

| | | |
|---|---|---|
| AIC | BIC | logLik |
| 5356.487 | 5382.888 | −2673.244 |

Random effects:
Formula: ~1|HAPMAP49592BTA38891

| | | |
|---|---|---|
| | (Intercept) | Residual |
| StdDev: | 0.05044103 | 1.517269 |

| | | | | | |
|---|---|---|---|---|---|
| Fixed effects: list(fixed) | | | | | |
| | Value | Std. Error | DF | t-value | p-value |
| (Intercept) | 3.0143927 | 0.07299842 | 1449 | 41.29395 | 0 |
| ARSBFGLNGS68110C/A | −0.5394633 | 0.08713974 | 1449 | −6.19078 | 0 |
| ARSBFGLNGS68110A/A | −0.5750699 | 0.11692351 | 1449 | −4.91834 | 0 |

| | | |
|---|---|---|
| Correlation: | | |
| | (Intr) | ARSBFGLNGS68110C |
| ARSBFGLNGS68110C/A | −0.630 | |
| ARSBFGLNGS68110A/A | −0.474 | 0.392 |

Standardized Within-Group Residuals:

| | | | | |
|---|---|---|---|---|
| Min | Q1 | Med | Q3 | Max |
| −1.9390075 | −0.7268338 | −0.1061961 | 0.5915021 | 4.6776139 |

Number of Observations: 1454
Number of Groups: 3

| | | | | |
|---|---|---|---|---|
| | numDf | denDF | F-value | p-value |
| (Intercept) | 1 | 1449 | 2485.3752 | <.0001 |
| ARSBFGLNGS68110 | 2 | 1449 | 22.8358 | <.0001 |

ARSBFGLNGS68110 × ARSBFGLNGS30157
Linear mixed-effects model fit by REML
Data: vm

| | | |
|---|---|---|
| AIC | BIC | logLik |
| 5353.322 | 5379.722 | −2671.661 |

Random effects:
Formula: ~1|ARSBFGLNGS30157

| | | |
|---|---|---|
| | (Intercept) | Residual |
| StdDev: | 0.1541638 | 1.514890 |

| | | | | | |
|---|---|---|---|---|---|
| Fixed effects: list(fixed) | | | | | |
| | Value | Std. Error | DF | t-value | p-value |
| (Intercept) | 3.0880895 | 0.12485023 | 1449 | 24.734353 | 0 |
| ARSBFGLNGS68110C/A | −0.5374762 | 0.08702799 | 1449 | −6.175900 | 0 |
| ARSBFGLNG568110A/A | −0.5597842 | 0.11686764 | 1449 | −4.789899 | 0 |

| | | |
|---|---|---|
| Correlation: | | |
| | (Intr) | ARSBFGLNGS68110C |
| ARSBFGLNGS68110C/A | −0.353 | |
| ARSBFGLNGS68110A/A | −0.254 | 0.392 |

Standardized Within-Group Residuals:

| | | | | |
|---|---|---|---|---|
| Min | Q1 | Med | Q3 | Max |
| −1.9556466 | −0.7300957 | −0.1094464 | 0.6166788 | 4.7093841 |

Number of Observations: 1454
Number of Groups: 3

| | | | | |
|---|---|---|---|---|
| | numDf | denDF | F-value | p-value |
| (Intercept) | 1 | 1449 | 574.9836 | <.0001 |
| ARSBFGLNGS68110 | 2 | 1449 | 22.3869 | <.0001 |

ARSBFGLNGS68110 × HAPMAP30097BTC007678
Linear mixed-effects model fit by REML
Data: vm

| | | |
|---|---|---|
| AIC | BIC | logLik |
| 5356.577 | 5382.977 | −2673.288 |

Random effects:
Formula: ~1|HAPMAP30097BTC007678

| | | |
|---|---|---|
| | (Intercept) | Residual |
| StdDev: | 9.713153e−05 | 1.517608 |

| | | | | | |
|---|---|---|---|---|---|
| Fixed effects: list(fixed) | | | | | |
| | Value | Std. Error | DF | t-value | p-value |
| (Intercept) | 3.0135182 | 0.06317891 | 1449 | 47.69817 | 0 |
| ARSBFGLNGS68110C/A | −0.5399658 | 0.08715416 | 1449 | −6.19552 | 0 |
| ARSBFGLNGS68110A/A | −0.5736022 | 0.11691281 | 1449 | −4.90624 | 0 |

| | | |
|---|---|---|
| Correlation: | | |
| | (Intr) | ARSBFGLNGS68110C |
| ARSBFGLNGS68110C/A | −0.725 | |
| ARSBFGLNGS68110A/A | −0.540 | 0.392 |

Standardized Within-Group Residuals:

| | | | | |
|---|---|---|---|---|
| Min | Q1 | Med | Q3 | Max |
| −1.9198092 | −0.7337323 | −0.1032771 | 0.6104655 | 4.6695068 |

Number of Observations: 1454
Number of Groups: 3

| | | | | |
|---|---|---|---|---|
| | numDf | denDF | F-value | p-value |
| (Intercept) | 1 | 1449 | 4542.196 | <.0001 |
| ARSBFGLNGS68110 | 2 | 1449 | 22.823 | <.0001 |

HAPMAP49592BTA38891 × ARSBFGLNGS102860
Linear mixed-effects model fit by REML
Data: vm

| | | |
|---|---|---|
| AIC | BIC | logLik |
| 5395.082 | 5421.482 | −2692.541 |

Random effects:
Formula: ~1|ARSBFGLNGS102860

| | | |
|---|---|---|
| | (Intercept) | Residual |
| StdDev: | 0.0001715283 | 1.538746 |

| | | | | | |
|---|---|---|---|---|---|
| Fixed effects: list(fixed) | | | | | |
| | Value | Std. Error | DF | t-value | p-value |
| (Intercept) | 2.6651140 | 0.04844218 | 1449 | 55.01639 | 0.0000 |
| HAPMAP49592BTA38891C/T | 0.1056054 | 0.09067497 | 1449 | 1.16466 | 0.2443 |
| HAPMAP49592BTA38891T/T | −0.4174949 | 0.24232507 | 1449 | −1.72287 | 0.0851 |

| | | |
|---|---|---|
| Correlation: | | |
| | (Intr) | HAPMAP49592BTA38891C |
| HAPMAP49592BTA38891C/T | −0.534 | |
| HAPMAP49592BTA38891T/T | −0.200 | 0.107 |

Standardized Within-Group Residuals:

| | | | | |
|---|---|---|---|---|
| Min | Q1 | Med | Q3 | Max |
| −1.7356464 | −0.7571839 | −0.1073042 | 0.6075635 | 4.8317814 |

Number of Observations: 1454
Number of Groups: 3

| | | | | |
|---|---|---|---|---|
| | numDf | denDF | F-value | p-value |
| (Intercept) | 1 | 1449 | 4418.237 | <.0001 |
| HAPMAP49592BTA38891 | 2 | 1449 | 2.404 | 0.0907 |

HAPMAP49592BTA38891 × BFGLNGS119018
Linear mixed-effects model fit by REML
Data: vm

| | | |
|---|---|---|
| AIC | BIC | logLik |
| 5394.222 | 5420.622 | −2692.111 |

Random effects:
Formula: ~1|BFGLNGS119018

| | | |
|---|---|---|
| | (Intercept) | Residual |
| StdDev: | 0.08541753 | 1.537704 |

Fixed effects: list(fixed)

| | | | | | |
|---|---|---|---|---|---|
| | Value | Std. Error | DF | t-value | p-value |
| (Intercept) | 2.6352222 | 0.07784362 | 1449 | 33.85277 | 0.0000 |
| HAPMAP49592BTA38891C/T | 0.1092921 | 0.09065146 | 1449 | 1.20563 | 0.2282 |
| HAPMAP49592BTA38891T/T | −0.4096887 | 0.24222485 | 1449 | −1.69136 | 0.0910 |
| Correlation: | | | | | |

| | | |
|---|---|---|
| | (Intr) | HAPMAP49592BTA38891C |
| HAPMAP49592BTA38891C/T | −0.340 | |
| HAPMAP49592BTA38891T/T | −0.130 | 0.107 |

Standardized Within-Group Residuals:

| | | | | |
|---|---|---|---|---|
| Min | Q1 | Med | Q3 | Max |
| −1.7558773 | −0.7723418 | −0.1240343 | 0.5933303 | 4.8183981 |

Number of Observations: 1454
Number of Groups: 3

| | | | | |
|---|---|---|---|---|
| | numDF | denDF | F-value | p-value |
| (Intercept) | 1 | 1449 | 1327.7109 | <.0001 |
| HAPMAP49592BTA38891 | 2 | 1449 | 2.4038 | 0.0907 |

HAPMAP49592BTA38891 × ARSBFGLBAC20850
Linear mixed-effects model fit by REML
Data: vm

| | | |
|---|---|---|
| AIC | BIC | logLik |
| 5395.082 | 5421.482 | −2692.541 |

Random effects:
Formula: ~1|ARSBFGLBAC20850

| | | |
|---|---|---|
| | (Intercept) | Residual |
| StdDev: | 9.52601e−05 | 1.538746 |

Fixed effects: list(fixed)

| | | | | | |
|---|---|---|---|---|---|
| | Value | Std. Error | DF | t-value | p-value |
| (Intercept) | 2.6651140 | 0.04844200 | 1449 | 55.01659 | 0.0000 |
| HAPMAP49592BTA38891C/T | 0.1056056 | 0.09067473 | 1449 | 1.16466 | 0.2443 |
| HAPMAP49592BTA38891T/T | −0.4174949 | 0.24232497 | 1449 | −1.72287 | 0.0851 |
| Correlation: | | | | | |

| | | |
|---|---|---|
| | (Intr) | HAPMAP49592BTA38891C |
| HAPMAP49592BTA38891C/T | −0.534 | |
| HAPMAP49592BTA38891T/T | −0.200 | 0.107 |

Standardized Within-Group Residuals:

| | | | | |
|---|---|---|---|---|
| Min | Q1 | Med | Q3 | Max |
| −1.7356464 | −0.7571839 | −0.1073042 | 0.6075634 | 4.8317814 |

Number of Observations: 1454
Number of Groups: 3

| | | | | |
|---|---|---|---|---|
| | numDF | denDF | F-value | p-value |
| (Intercept) | 1 | 1449 | 4418.259 | <.0001 |
| HAPMAP49592BTA38891 | 2 | 1449 | 2.404 | 0.0907 |

HAPMAP49592BTA38891 × ARSBFGLNGS100843
Linear mixed-effects model fit by REML
Data: vm

| | | |
|---|---|---|
| AIC | BIC | logLik |
| 5393.938 | 5420.338 | −2691.969 |

Random effects:
Formula: ~1|ARSBFGLNGS100843

| | | |
|---|---|---|
| | (Intercept) | Residual |
| StdDev: | 0.1293746 | 1.537407 |

Fixed effects: list(fixed)

| | | | | | |
|---|---|---|---|---|---|
| | Value | Std. Error | DF | t-value | p-value |
| (Intercept) | 2.7402474 | 0.10681496 | 1449 | 25.654153 | 0.0000 |
| HAPMAP49592BTA38891C/T | 0.1031760 | 0.09060867 | 1449 | 1.138699 | 0.2550 |
| HAPMAP49592BTA38891T/T | −0.4205069 | 0.24218950 | 1449 | −1.736272 | 0.0827 |
| Correlation: | | | | | |

| | | |
|---|---|---|
| | (Intr) | HAPMAP49592BTA38891C |
| HAPMAP49592BTA38891C/T | −0.249 | |
| HAPMAP49592BTA38891T/T | −0.102 | 0.107 |

Standardized Within-Group Residuals:

| | | | | |
|---|---|---|---|---|
| Min | Q1 | Med | Q3 | Max |
| −1.75257913 | −0.74399301 | −0.09561283 | 0.61987779 | 4.84984277 |

Number of Observations: 1454
Number of Groups: 3

| | | | | |
|---|---|---|---|---|
| | numDF | denDF | F-value | p-value |
| (Intercept) | 1 | 1449 | 713.9473 | <.0001 |
| HAPMAP49592BTA38891 | 2 | 1449 | 2.3943 | 0.0916 |

HAPMAP49592BTA38891 × ARSBFGLNGS97162
Linear mixed-effects model fit by REML
Data: vm

| | | |
|---|---|---|
| AIC | BIC | logLik |
| 5390.587 | 5416.987 | −2690.294 |

Random effects:
Formula: ~1|ARSBFGLNGS97162

| | | |
|---|---|---|
| | (Intercept) | Residual |
| StdDev: | 0.1682108 | 1.535194 |

Fixed effects: list(fixed)

| | | | | | |
|---|---|---|---|---|---|
| | Value | Std. Error | DF | t-value | p-value |
| (Intercept) | 2.7363491 | 0.12655535 | 1449 | 21.621759 | 0.0000 |
| HAPMAP49592BTA38891C/T | 0.1193719 | 0.09063391 | 1449 | 1.317078 | 0.1880 |
| HAPMAP49592BTA38891T/T | −0.3874695 | 0.24204622 | 1449 | −1.600808 | 0.1096 |
| Correlation: | | | | | |

| | | |
|---|---|---|
| | (Intr) | HAPMAP49592BTA38891C |
| HAPMAP49592BTA38891C/T | −0.199 | |
| HAPMAP49592BTA38891T/T | −0.068 | 0.109 |

Standardized Within-Group Residuals:

| | | | | |
|---|---|---|---|---|
| Min | Q1 | Med | Q3 | Max |
| −1.7675965 | −0.7287398 | −0.1298763 | 0.6391651 | 4.8857749 |

Number of Observations: 1454
Number of Groups: 3

| | | | | |
|---|---|---|---|---|
| | numDF | denDF | F-value | p-value |
| (Intercept) | 1 | 1449 | 495.9805 | <.0001 |
| HAPMAP49592BTA38891 | 2 | 1449 | 2.4080 | 0.0904 |

HAPMAP49592BTA38891 × Hapmap42294BTA69421
Linear mixed-effects model fit by REML
Data: vm

| | | |
|---|---|---|
| AIC | BIC | logLik |
| 5394.442 | 5420.842 | −2692.221 |

Random effects:
Formula: ~1|Hapmap42294BTA69421

| | | |
|---|---|---|
| | (Intercept) | Residual |
| StdDev: | 0.0752072 | 1.537644 |

Fixed effects: list(fixed)

| | | | | | |
|---|---|---|---|---|---|
| | Value | Std. Error | DF | t-value | p-value |
| (Intercept) | 2.6615976 | 0.06619226 | 1449 | 40.21011 | 0.0000 |
| HAPMAP49592BTA38891C/T | 0.1036428 | 0.09065462 | 1449 | 1.14327 | 0.2531 |
| HAPMAP49592BTA38891T/T | −0.4110896 | 0.24228336 | 1449 | −1.69673 | 0.0900 |
| Correlation: | | | | | |

| | | |
|---|---|---|
| | (Intr) | HAPMAP49592BTA38891C |
| HAPMAP49592BTA38891C/T | −0.395 | |
| HAPMAP49592BTA38891T/T | −0.152 | 0.107 |

Standardized Within-Group Residuals:

| | | | | |
|---|---|---|---|---|
| Min | Q1 | Med | Q3 | Max |
| −1.77281726 | −0.74228830 | −0.09194276 | 0.62343733 | 4.79804177 |

Number of Observations: 1454
Number of Groups: 3

| | | | | |
|---|---|---|---|---|
| | numDF | denDF | F-value | p-value |
| (Intercept) | 1 | 1449 | 1967.9541 | <.0001 |
| HAPMAP49592BTA38891 | 2 | 1449 | 2.3276 | 0.0979 |

HAPMAP49592BTA38891 × ARSBFGLBAC2384
Linear mixed-effects model fit by REML
Data: vm

| | | |
|---|---|---|
| AIC | BIC | logLik |
| 5393.456 | 5419.856 | −2691.728 |

Random effects:
Formula: ~1|ARSBFGLBAC2384

| | | |
|---|---|---|
| | (Intercept) | Residual |
| StdDev: | 0.08903045 | 1.537026 |

Fixed effects: list(fixed)

| | | | | | |
|---|---|---|---|---|---|
| | Value | Std. Error | DF | t-value | p-value |
| (Intercept) | 2.6620075 | 0.07410367 | 1449 | 35.92275 | 0.0000 |
| HAPMAP49592BTA38891C/T | 0.1084637 | 0.09063463 | 1449 | 1.19671 | 0.2316 |
| HAPMAP49592BTA38891T/T | −0.4094234 | 0.24209747 | 1449 | −1.69115 | 0.0910 |
| Correlation: | | | | | |

| | | |
|---|---|---|
| | (Intr) | HAPMAP49592BTA38891C |
| HAPMAP49592BTA38891C/T | −0.359 | |
| HAPMAP49592BTA38891T/T | −0.132 | 0.107 |

Standardized Within-Group Residuals:

| | | | | |
|---|---|---|---|---|
| Min | Q1 | Med | Q3 | Max |
| −1.78431526 | −0.77592439 | −0.09280144 | 0.62843702 | 4.79232158 |

Number of Observations: 1454
Number of Groups: 3

| | | | | |
|---|---|---|---|---|
| | numDF | denDF | F-value | p-value |
| (Intercept) | 1 | 1449 | 1517.8818 | <.0001 |
| HAPMAP49592BTA38891 | 2 | 1449 | 2.3904 | 0.092 |

HAPMAP49592BTA38891 × BTB01553536
Linear mixed-effects model fit by REML
Data: vm

| | | |
|---|---|---|
| AIC | BIC | logLik |
| 5393.375 | 5419.775 | −2691.688 |

Random effects:
Formula: ~1|BTB01553536

| | | |
|---|---|---|
| | (Intercept) | Residual |
| StdDev: | 0.08934136 | 1.536859 |

Fixed effects: list(fixed)

| | | | | | |
|---|---|---|---|---|---|
| | Value | Std. Error | DF | t-value | p-value |
| (Intercept) | 2.6672618 | 0.07131045 | 1449 | 37.40352 | 0.0000 |
| HAPMAP49592BTA38891C/T | 0.1040588 | 0.09069020 | 1449 | 1.14741 | 0.2514 |
| HAPMAP49592BTA38891T/T | −0.4116865 | 0.24205090 | 1449 | −1.70083 | 0.0892 |
| Correlation: | | | | | |

| | | |
|---|---|---|
| | (Intr) | HAPMAP49592BTA38891C |
| HAPMAP49592BTA38891C/T | −0.355 | |
| HAPMAP49592BTA38891T/T | −0.135 | 0.107 |

Standardized Within-Group Residuals:

| | | | | |
|---|---|---|---|---|
| Min | Q1 | Med | Q3 | Max |
| −1.7423978 | −0.7371729 | −0.1157033 | 0.6266095 | 4.7935884 |

Number of Observations: 1454
Number of Groups: 3

| | | | | |
|---|---|---|---|---|
| | numDF | denDF | F-value | p-value |
| (Intercept) | 1 | 1449 | 1638.8522 | <.0001 |
| HAPMAP49592BTA38891 | 2 | 1449 | 2.3398 | 0.0967 |

HAPMAP49592BTA38891 × HAPMAP53129RS29022984
Linear mixed-effects model fit by REML
Data: vm

| | | |
|---|---|---|
| AIC | BIC | logLik |
| 5388.901 | 5415.301 | −2689.450 |

Random effects:
Formula: ~1|HAPMAP53129RS29022984

| | | |
|---|---|---|
| | (Intercept) | Residual |
| StdDev: | 0.1696786 | 1.534194 |

Fixed effects: list(fixed)

| | | | | | |
|---|---|---|---|---|---|
| | Value | Std. Error | DF | t-value | p-value |
| (Intercept) | 2.5904400 | 0.1224497 | 1449 | 21.155138 | 0.0000 |
| HAPMAP49592BTA38891C/T | 0.0993440 | 0.0904380 | 1449 | 1.098476 | 0.2722 |
| HAPMAP49592BTA38891T/T | −0.4123969 | 0.2416144 | 1449 | −1.706839 | 0.0881 |
| Correlation: | | | | | |

| | | |
|---|---|---|
| | (Intr) | HAPMAP49592BTA38891C |
| HAPMAP49592BTA38891C/T | −0.211 | |
| HAPMAP49592BTA38891T/T | −0.081 | 0.107 |

Standardized Within-Group Residuals:

| | | | | |
|---|---|---|---|---|
| Min | Q1 | Med | Q3 | Max |
| −1.7769191 | −0.7344541 | −0.1046870 | 0.6288323 | 4.8059131 |

Number of Observations: 1454
Number of Groups: 3

| | | | | |
|---|---|---|---|---|
| | numDF | denDF | F-value | p-value |
| (Intercept) | 1 | 1449 | 475.4978 | <.0001 |
| HAPMAP49592BTA38891 | 2 | 1449 | 2.2858 | 0.1021 |

HAPMAP49592BTA38891 × ARSBFGLNGS68110
Linear mixed-effects model fit by REML
Data: vm

| | | |
|---|---|---|
| AIC | BIC | logLik |
| 5358.828 | 5385.228 | −2674.414 |

Random effects:
Formula: ~1|ARSBFGLNGS68110

| | | |
|---|---|---|
| | (Intercept) | Residual |
| StdDev: | 0.3126819 | 1.516556 |

Fixed effects: list(fixed)

| | | | | | |
|---|---|---|---|---|---|
| | Value | Std. Error | DF | t-value | p-value |
| (Intercept) | 2.6245660 | 0.18767458 | 1449 | 13.984664 | 0.0000 |
| HAPMAP49592BTA38891C/T | 0.1109898 | 0.08943642 | 1449 | 1.240991 | 0.2148 |
| HAPMAP49592BTA38891T/T | −0.3433260 | 0.23910818 | 1449 | −1.435860 | 0.1513 |
| Correlation: | | | | | |

| | | |
|---|---|---|
| | (Intr) | HAPMAP49592BTA38891C |
| HAPMAP49592BTA38891C/T | −0.140 | |
| HAPMAP49592BTA38891T/T | −0.053 | 0.107 |

Standardized Within-Group Residuals:

| | | | | |
|---|---|---|---|---|
| Min | Q1 | Med | Q3 | Max |
| −1.9690500 | −0.7089645 | −0.1068231 | 0.6098133 | 4.6980245 |

Number of Observations: 1454
Number of Groups: 3

| | | | | |
|---|---|---|---|---|
| | numDF | denDF | F-value | p-value |
| (Intercept) | 1 | 1449 | 203.0006 | <.0001 |
| HAPMAP49592BTA38891 | 2 | 1449 | 2.0154 | 0.1336 |

HAPMAP49592BTA38891 × HAPMAP49592BTA38891
Linear mixed-effects model fit by REML
Data: vm

| | | |
|---|---|---|
| AIC | BIC | logLik |
| 5395.082 | 5421.482 | −2692.541 |

Random effects:
Formula: ~1|HAPMAP49592BTA38891

| | | |
|---|---|---|
| | (Intercept) | Residual |
| StdDev: | 0.1863864 | 1.538746 |

Fixed effects: list(fixed)

| | | | | | |
|---|---|---|---|---|---|
| | Value | Std. Error | DF | t-value | p-value |
| (Intercept) | 2.6651140 | 0.1925786 | 1451 | 13.839099 | 0 |
| HAPMAP49592BTA38891C/T | 0.1056056 | 0.2787502 | 0 | 0.378854 | NaN |
| HAPMAP49592BTA38891T/T | −0.4174949 | 0.3580519 | 0 | −1.166018 | NaN |
| Correlation: | | | | | |

| | | |
|---|---|---|
| | (Intr) | HAPMAP49592BTA38891C |
| HAPMAP49592BTA38891C/T | −0.691 | |
| HAPMAP49592BTA38891T/T | −0.538 | 0.372 |

Standardized Within-Group Residuals:

| | | | | |
|---|---|---|---|---|
| Min | Q1 | Med | Q3 | Max |
| −1.7356464 | −0.7571839 | −0.1073042 | 0.6075634 | 4.8317814 |

Number of Observations: 1454
Number of Groups: 3

| | | | | |
|---|---|---|---|---|
| | numDF | denDF | F-value | p-value |
| (Intercept) | 1 | 1451 | 433.8555 | <.0001 |
| HAPMAP49592BTA38891 | 2 | 0 | 1.0624 | NaN |

HAPMAP49592BTA38891 × ARSBFGLNGS30157
Linear mixed-effects model fit by REML
Data: vm

| | | |
|---|---|---|
| AIC | BIC | logLik |
| 5390.916 | 5417.316 | −2690.458 |

Random effects:
Formula: ~1|ARSBFGLNGS30157

| | | |
|---|---|---|
| | (Intercept) | Residual |
| StdDev: | 0.1937946 | 1.535277 |

Fixed effects: list(fixed)

| | | | | | |
|---|---|---|---|---|---|
| | Value | Std. Error | DF | t-value | p-value |
| (Intercept) | 2.7788339 | 0.14293786 | 1449 | 19.440853 | 0.0000 |
| HAPMAP49592BTA38891C/T | 0.0966774 | 0.09054976 | 1449 | 1.067671 | 0.2858 |
| HAPMAP49592BTA38891T/T | −0.4335786 | 0.24188484 | 1449 | −1.792500 | 0.0733 |
| Correlation: | | | | | |

| | | |
|---|---|---|
| | (Intr) | HAPMAP49592BTA38891C |
| HAPMAP49592BTA38891C/T | −0.185 | |
| HAPMAP49592BTA38891T/T | −0.070 | 0.108 |

Standardized Within-Group Residuals:

| | | | | |
|---|---|---|---|---|
| Min | Q1 | Med | Q3 | Max |
| −1.78587884 | −0.74155766 | −0.09020954 | 0.64030846 | 4.87407122 |

Number of Observations: 1454
Number of Groups: 3

| | | | | |
|---|---|---|---|---|
| | numDF | denDF | F-value | p-value |
| (Intercept) | 1 | 1449 | 396.5671 | <.0001 |
| HAPMAP49592BTA38891 | 2 | 1449 | 2.4110 | 0.0901 |

HAPMAP49592BTA38891 × HAPMAP30097BTC007678
Linear mixed-effects model fit by REML
Data: vm

| | | |
|---|---|---|
| AIC | BIC | logLik |
| 5395.082 | 5421.482 | −2692.541 |

Random effects:
Formula: ~1|HAPMAP30097BTC007678

| | | |
|---|---|---|
| | (Intercept) | Residual |
| StdDev: | 0.0001168907 | 1.538746 |

Fixed effects: list(fixed)

| | | | | | |
|---|---|---|---|---|---|
| | Value | Std. Error | DF | t-value | p-value |
| (Intercept) | 2.6651140 | 0.04844204 | 1449 | 55.01656 | 0.0000 |
| HAPMAP49592BTA38891C/T | 0.1056056 | 0.09067473 | 1449 | 1.16466 | 0.2443 |
| HAPMAP49592BTA38891T/T | −0.4174949 | 0.24232497 | 1449 | −1.72287 | 0.0851 |
| Correlation: | | | | | |

| | | |
|---|---|---|
| | (Intr) | HAPMAP49592BTA38891C |
| HAPMAP49592BTA38891C/T | −0.534 | |
| HAPMAP49592BTA38891T/T | −0.200 | 0.107 |

Standardized Within-Group Residuals:

| | | | | |
|---|---|---|---|---|
| Min | Q1 | Med | Q3 | Max |
| −1.7356465 | −0.7571839 | −0.1073042 | 0.6075634 | 4.8317814 |

Number of Observations: 1454
Number of Groups: 3

| | | | | |
|---|---|---|---|---|
| | numDF | denDF | F-value | p-value |
| (Intercept) | 1 | 1449 | 4418.251 | <.0001 |
| HAPMAP49592BTA38891 | 2 | 1449 | 2.404 | 0.0907 |

ARSBFGLNGS30157 × ARSBFGLNGS102860
Linear mixed-effects model fit by REML
Data: vm

| | | |
|---|---|---|
| AIC | BIC | logLik |
| 5387.793 | 5414.193 | −2688.896 |

Random effects:
Formula: ~1|ARSBFGLNGS102860

| | | |
|---|---|---|
| | (Intercept) | Residual |
| StdDev: | 0.1746021 | 1.534188 |

Fixed effects: list(fixed)

| | | | | | |
|---|---|---|---|---|---|
| | Value | Std. Error | DF | t-value | p-value |
| (Intercept) | 2.5650707 | 0.1192271 | 1449 | 21.514153 | 0.0000 |
| ARSBFGLNGS30157G/A | 0.2486345 | 0.0972671 | 1449 | 2.556205 | 0.0107 |
| ARSBFGLNGS30157A/A | 0.9692205 | 0.5135652 | 1449 | 1.887239 | 0.0593 |
| Correlation: | | | | | |

| | | |
|---|---|---|
| | (Intr) | ARSBFGLNGS30157G |
| ARSBFGLNGS30157G/A | −0.194 | |
| ARSBFGLNGS30157A/A | −0.029 | 0.041 |

Standardized Within-Group Residuals:

| | | | | |
|---|---|---|---|---|
| Min | Q1 | Med | Q3 | Max |
| −1.79081610 | −0.74791923 | −0.09209587 | 0.62088290 | 4.88935190 |

Number of Observations: 1454
Number of Groups: 3

| | | | | |
|---|---|---|---|---|
| | numDF | denDF | F-value | p-value |
| (Intercept) | 1 | 1449 | 505.1641 | <.0001 |
| ARSBFGLNGS30157 | 2 | 1449 | 4.8576 | 0.0079 |

ARSBFGLNGS30157 × BFGLNGS119018
Linear mixed-effects model fit by REML
Data: vm

| | | |
|---|---|---|
| AIC | BIC | logLik |
| 5387.924 | 5414.324 | −2688.962 |

Random effects:
Formula: ~1|BFGLNGS119018

| | | |
|---|---|---|
| | (Intercept) | Residual |
| StdDev: | 0.08290932 | 1.535272 |

Fixed effects: list(fixed)

| | | | | | |
|---|---|---|---|---|---|
| | Value | Std. Error | DF | t-value | p-value |
| (Intercept) | 2.5950942 | 0.0746611 | 1449 | 34.75833 | 0.0000 |
| ARSBFGLNGS30157G/A | 0.2463088 | 0.0972682 | 1449 | 2.53226 | 0.0114 |
| ARSBFGLNGS30157A/A | 0.9518984 | 0.5139153 | 1449 | 1.85225 | 0.0642 |
| Correlation: | | | | | |

| | | |
|---|---|---|
| | (Intr) | ARSBFGLNGS30157G |
| ARSBFGLNGS30157G/A | −0.289 | |
| ARSBFGLNGS30157A/A | −0.048 | 0.042 |

Standardized Within-Group Residuals:

| | | | | |
|---|---|---|---|---|
| Min | Q1 | Med | Q3 | Max |
| −1.82006595 | −0.74774187 | −0.09639136 | 0.62009420 | 4.85387252 |

Number of Observations: 1454
Number of Groups: 3

| | | | | |
|---|---|---|---|---|
| | numDF | denDF | F-value | p-value |
| (Intercept) | 1 | 1449 | 1380.7832 | <.0001 |
| ARSBFGLNGS30157 | 2 | 1449 | 4.7336 | 0.0089 |

ARSBFGLNGS30157 × ARSBFGLBAC20850
Linear mixed-effects model fit by REML
Data: vm

| | | |
|---|---|---|
| AIC | BIC | logLik |
| 5388.715 | 5415.115 | −2689.357 |

Random effects:
Formula: ~1|ARSBFGLBAC20850

| | | |
|---|---|---|
| | (Intercept) | Residual |
| StdDev: | 9.425869e−05 | 1.536253 |

Fixed effects: list(fixed)

| | | | | | |
|---|---|---|---|---|---|
| | Value | Std. Error | DF | t-value | p-value |
| (Intercept) | 2.6222222 | 0.0458023 | 1449 | 57.25089 | 0.0000 |
| ARSBFGLNGS30157G/A | 0.2459028 | 0.0973297 | 1449 | 2.52649 | 0.0116 |
| ARSBFGLNGS30157A/A | 0.9666667 | 0.5141287 | 1449 | 1.88020 | 0.0603 |
| Correlation: | | | | | |

| | | |
|---|---|---|
| | (Intr) | ARSBFGLNGS30157G |
| ARSBFGLNGS30157G/A | −0.471 | |
| ARSBFGLNGS30157A/A | −0.089 | 0.042 |

Standardized Within-Group Residuals:

| | | | | |
|---|---|---|---|---|
| Min | Q1 | Med | Q3 | Max |
| −1.80186743 | −0.73049290 | −0.07955863 | 0.63646906 | 4.86754182 |

Number of Observations: 1454
Number of Groups: 3

| | | | | |
|---|---|---|---|---|
| | numDF | denDF | F-value | p-value |
| (Intercept) | 1 | 1449 | 4432.611 | <.0001 |
| ARSBFGLNGS30157 | 2 | 1449 | 4.768 | 0.0086 |

ARSBFGLNGS30157 × ARSBFGLNGS100843
Linear mixed-effects model fit by REML
Data: vm

| | | |
|---|---|---|
| AIC | BIC | logLik |
| 5388.085 | 5414.485 | −2689.043 |

Random effects:
Formula: ~1|ARSBFGLNGS100843

| | | |
|---|---|---|
| | (Intercept) | Residual |
| StdDev: | 0.1049622 | 1.535360 |

Fixed effects: list(fixed)

| | | | | | |
|---|---|---|---|---|---|
| | Value | Std. Error | DF | t-value | p-value |
| (Intercept) | 2.6786297 | 0.0918071 | 1449 | 29.176717 | 0.0000 |
| ARSBFGLNGS30157G/A | 0.2393433 | 0.0974261 | 1449 | 2.456666 | 0.0141 |
| ARSBFGLNGS30157A/A | 0.9442775 | 0.5141632 | 1449 | 1.836533 | 0.0665 |
| Correlation: | | | | | |

| | | |
|---|---|---|
| | (Intr) | ARSBFGLNGS30157G |
| ARSBFGLNGS30157G/A | −0.252 | |
| ARSBFGLNGS30157A/A | −0.056 | 0.044 |

Standardized Within-Group Residuals:

| | | | | |
|---|---|---|---|---|
| Min | Q1 | Med | Q3 | Max |
| −1.78871095 | −0.74230740 | −0.09529665 | 0.62034443 | 4.88030843 |

Number of Observations: 1454
Number of Groups: 3

| | | | | |
|---|---|---|---|---|
| | numDF | denDF | F-value | p-value |
| (Intercept) | 1 | 1449 | 955.2355 | <.0001 |
| ARSBFGLNGS30157 | 2 | 1449 | 4.5149 | 0.0111 |

ARSBFGLNGS30157 × ARSBFGLNGS97162
Linear mixed-effects model fit by REML
Data: vm

| | | |
|---|---|---|
| AIC | BIC | logLik |
| 5383.452 | 5409.852 | −2686.726 |

Random effects:
Formula: ~1|ARSBFGLNGS97162

| | | |
|---|---|---|
| | (Intercept) | Residual |
| StdDev: | 0.1840609 | 1.532209 |

Fixed effects: list(fixed)

| | | | | | |
|---|---|---|---|---|---|
| | Value | Std. Error | DF | t-value | p-value |
| (Intercept) | 2.7040507 | 0.1351555 | 1449 | 20.006955 | 0.0000 |
| ARSBFGLNGS30157G/A | 0.2731564 | 0.0975814 | 1449 | 2.799267 | 0.0052 |
| ARSBFGLNGS30157A/A | 0.8580415 | 0.5143633 | 1449 | 1.668162 | 0.0955 |
| Correlation: | | | | | |

| | | |
|---|---|---|
| | (Intr) | ARSBFGLNGS30157G |
| ARSBFGLNGS30157G/A | −0.143 | |
| ARSBFGLNGS30157A/A | −0.038 | 0.034 |

Standardized Within-Group Residuals:

| | | | | |
|---|---|---|---|---|
| Min | Q1 | Med | Q3 | Max |
| −1.92886666 | −0.75111669 | −0.09846416 | 0.61945363 | 4.92696035 |

Number of Observations: 1454
Number of Groups: 3

| | | | | |
|---|---|---|---|---|
| | numDF | denDF | F-value | p-value |
| (Intercept) | 1 | 1449 | 427.8136 | <.0001 |
| ARSBFGLNGS30157 | 2 | 1449 | 5.1582 | 0.0059 |

ARSBFGLNGS30157 × Hapmap42294BTA69421
Linear mixed-effects model fit by REML
Data: vm

| | | |
|---|---|---|
| AIC | BIC | logLik |
| 5388.015 | 5414.415 | −2689.008 |

Random effects:
Formula: ~1|Hapmap42294BTA69421

| | | |
|---|---|---|
| | (Intercept) | Residual |
| StdDev: | 0.07767022 | 1.535084 |

Fixed effects: list(fixed)

| | | | | | |
|---|---|---|---|---|---|
| | Value | Std. Error | DF | t-value | p-value |
| (Intercept) | 2.6178095 | 0.0651422 | 1449 | 40.18605 | 0.0000 |
| ARSBFGLNGS30157G/A | 0.2463575 | 0.0972561 | 1449 | 2.53308 | 0.0114 |
| ARSBFGLNGS30157A/A | 0.9472895 | 0.5139159 | 1449 | 1.84328 | 0.0655 |
| Correlation: | | | | | |

| | | |
|---|---|---|
| | (Intr) | ARSBFGLNGS30157G |
| ARSBFGLNGS30157G/A | −0.331 | |
| ARSBFGLNGS30157A/A | −0.063 | 0.042 |

Standardized Within-Group Residuals:

| | | | | |
|---|---|---|---|---|
| Min | Q1 | Med | Q3 | Max |
| −1.7877266 | −0.7649160 | −0.0940083 | 0.6030873 | 4.8327875 |

Number of Observations: 1454
Number of Groups: 3

| | | | | |
|---|---|---|---|---|
| | numDF | denDF | F-value | p-value |
| (Intercept) | 1 | 1449 | 1902.649 | <.0001 |
| ARSBFGLNGS30157 | 2 | 1449 | 4.720 | 0.0091 |

ARSBFGLNGS30157 × ARSBFGLBAC2384
Linear mixed-effects model fit by REML
Data: vm

| | | |
|---|---|---|
| AIC | BIC | logLik |
| 5386.904 | 5413.304 | −2688.452 |

Random effects:
Formula: ~1|ARSBFGLBAC2384

| | | |
|---|---|---|
| | (Intercept) | Residual |
| StdDev: | 0.09350635 | 1.534383 |

Fixed effects: list(fixed)

| | | | | | |
|---|---|---|---|---|---|
| | Value | Std. Error | DF | t-value | p-value |
| (Intercept) | 2.6163887 | 0.0744595 | 1449 | 35.13839 | 0.0000 |
| ARSBFGLNGS30157G/A | 0.2548752 | 0.0973810 | 1449 | 2.61730 | 0.0090 |
| ARSBFGLNGS30157A/A | 0.9202338 | 0.5141685 | 1449 | 1.78975 | 0.0737 |
| Correlation: | | | | | |

| | | |
|---|---|---|
| | (Intr) | ARSBFGLNGS30157G |
| ARSBFGLNGS30157G/A | −0.301 | |
| ARSBFGLNGS30157A/A | −0.058 | 0.040 |

Standardized Within-Group Residuals:

| | | | | |
|---|---|---|---|---|
| Min | Q1 | Med | Q3 | Max |
| −1.8572043 | −0.7583690 | −0.1066414 | 0.6102589 | 4.8261807 |

Number of Observations: 1454
Number of Groups: 3

| | | | | |
|---|---|---|---|---|
| | numDF | denDF | F-value | p-value |
| (Intercept) | 1 | 1449 | 1428.4856 | <.0001 |
| ARSBFGLNGS30157 | 2 | 1449 | 4.8471 | 0.008 |

ARSBFGLNGS30157 × BTB01553536
Linear mixed-effects model fit by REML
Data: vm

| | | |
|---|---|---|
| AIC | BIC | logLik |
| 5386.45 | 5412.85 | −2688.225 |

Random effects:
Formula: ~1|BTB01553536

| | | |
|---|---|---|
| | (Intercept) | Residual |
| StdDev: | 0.09849294 | 1.533945 |

Fixed effects: list(fixed)

| | | | | | |
|---|---|---|---|---|---|
| | Value | Std. Error | DF | t-value | p-value |
| (Intercept) | 2.6238476 | 0.0735907 | 1449 | 35.65462 | 0.0000 |
| ARSBFGLNGS30157G/A | 0.2543350 | 0.0974397 | 1449 | 2.61018 | 0.0091 |
| ARSBFGLNGS30157A/A | 0.9696553 | 0.5134161 | 1449 | 1.88863 | 0.0591 |
| Correlation: | | | | | |

| | | |
|---|---|---|
| | (Intr) | ARSBFGLNGS30157G |
| ARSBFGLNGS30157G/A | −0.284 | |
| ARSBFGLNGS30157A/A | −0.053 | 0.043 |

Standardized Within-Group Residuals:

| | | | | |
|---|---|---|---|---|
| Min | Q1 | Med | Q3 | Max |
| −1.8575745 | −0.7423807 | −0.1271766 | 0.6266385 | 4.8273691 |

Number of Observations: 1454
Number of Groups: 3

| | | | | |
|---|---|---|---|---|
| | numDF | denDF | F-value | p-value |
| (Intercept) | 1 | 1449 | 1448.9257 | <.0001 |
| ARSBFGLNGS30157 | 2 | 1449 | 4.9881 | 0.0069 |

ARSBFGLNGS30157 × HAPMAP53129RS29022984
Linear mixed-effects model fit by REML
Data: vm

| | | |
|---|---|---|
| AIC | BIC | logLik |
| 5382.688 | 5409.088 | −2686.344 |

Random effects:
Formula: ~1|HAPMAP53129RS29022984

| | | |
|---|---|---|
| | (Intercept) | Residual |
| StdDev: | 0.1681216 | 1.531801 |

Fixed effects: list(fixed)

| | | | | | |
|---|---|---|---|---|---|
| | Value | Std. Error | DF | t-value | p-value |
| (Intercept) | 2.5445161 | 0.1206457 | 1449 | 21.090814 | 0.0000 |
| ARSBFGLNGS30157G/A | 0.2430924 | 0.0970601 | 1449 | 2.504556 | 0.0124 |
| ARSBFGLNGS30157A/A | 0.9232005 | 0.5131144 | 1449 | 1.799210 | 0.0722 |

Correlation:

| | | |
|---|---|---|
| | (Intr) | ARSBFGLNGS30157G |
| ARSBFGLNGS30157G/A | −0.181 | |
| ARSBFGLNGS30157A/A | −0.040 | 0.043 |

Standardized Within-Group Residuals:

| | | | | |
|---|---|---|---|---|
| Min | Q1 | Med | Q3 | Max |
| −1.8446667 | −0.7348621 | −0.1191867 | 0.5989221 | 4.8422926 |

Number of Observations: 1454
Number of Groups: 3

| | | | | |
|---|---|---|---|---|
| | numDF | denDF | F-value | p-value |
| (Intercept) | 1 | 1449 | 482.8002 | <.0001 |
| ARSBFGLNGS30157 | 2 | 1449 | 4.5715 | 0.0105 |

ARSBFGLNGS30157 × ARSBFGLNGS68110
Linear mixed-effects model fit by REML
Data: vm

| | | |
|---|---|---|
| AIC | BIC | logLik |
| 5353.751 | 5380.151 | −2671.875 |

Random effects:
Formula: ~1|ARSBFGLNGS68110

| | | |
|---|---|---|
| | (Intercept) | Residual |
| StdDev: | 0.3055576 | 1.514818 |

Fixed effects: list(fixed)

| | | | | | |
|---|---|---|---|---|---|
| | Value | Std. Error | DF | t-value | p-value |
| (Intercept) | 2.5933368 | 0.1828450 | 1449 | 14.183254 | 0.0000 |
| ARSBFGLNGS30157G/A | 0.2312245 | 0.0960959 | 1449 | 2.406186 | 0.0162 |
| ARSBFGLNGS30157A/A | 0.7097344 | 0.5085382 | 1449 | 1.395636 | 0.1630 |

Correlation:

| | | |
|---|---|---|
| | (Intr) | ARSBFGLNGS30157G |
| ARSBFGLNGS30157G/A | −0.112 | |
| ARSBFGLNGS30157A/A | −0.019 | 0.044 |

Standardized Within-Group Residuals:

| | | | | |
|---|---|---|---|---|
| Min | Q1 | Med | Q3 | Max |
| −1.9586629 | −0.7381559 | −0.1038210 | 0.6365167 | 4.7294179 |

Number of Observations: 1454
Number of Groups: 3

| | | | | |
|---|---|---|---|---|
| | numDF | denDF | F-value | p-value |
| (Intercept) | 1 | 1449 | 212.08501 | <.0001 |
| ARSBFGLNGS30157 | 2 | 1449 | 3.72852 | 0.0243 |

ARSBFGLNGS30157 × HAPMAP49592BTA38891
Linear mixed-effects model fit by REML
Data: vm

| | | |
|---|---|---|
| AIC | BIC | logLik |
| 5388.7 | 5415.1 | −2689.35 |

Random effects:
Formula: ~1|HAPMAP49592BTA38891

| | | |
|---|---|---|
| | (Intercept) | Residual |
| StdDev: | 0.03476756 | 1.536092 |

Fixed effects: list(fixed)

| | | | | | |
|---|---|---|---|---|---|
| | Value | Std. Error | DF | t-value | p-value |
| (Intercept) | 2.6220892 | 0.0524749 | 1449 | 49.96844 | 0.0000 |
| ARSBFGLNGS30157G/A | 0.2450956 | 0.0973448 | 1449 | 2.51781 | 0.0119 |
| ARSBFGLNGS30157A/A | 0.9674097 | 0.5140822 | 1449 | 1.88182 | 0.0601 |

Correlation:

| | | |
|---|---|---|
| | (Intr) | ARSBFGLNGS30157G |
| ARSBFGLNGS30157G/A | −0.416 | |
| ARSBFGLNGS30157A/A | −0.076 | 0.042 |

Standardized Within-Group Residuals:

| | | | | |
|---|---|---|---|---|
| Min | Q1 | Med | Q3 | Max |
| −1.7982919 | −0.7397306 | −0.0887280 | 0.6273749 | 4.8712923 |

Number of Observations: 1454
Number of Groups: 3

| | | | | |
|---|---|---|---|---|
| | numDF | denDF | F-value | p-value |
| (Intercept) | 1 | 1449 | 3172.646 | <.0001 |
| ARSBFGLNGS30157 | 2 | 1449 | 4.751 | 0.0088 |

ARSBFGLNGS30157 × ARSBFGLNGS30157
Linear mixed-effects model fit by REML
Data: vm

| | | |
|---|---|---|
| AIC | BIC | logLik |
| 5388.715 | 5415.115 | −2689.357 |

Random effects:
Formula: ~1|ARSBFGLNGS30157

| | | |
|---|---|---|
| | (Intercept) | Residual |
| StdDev: | 0.1860844 | 1.536253 |

Fixed effects: list(fixed)

| | | | | | |
|---|---|---|---|---|---|
| | Value | Std. Error | DF | t-value | p-value |
| (Intercept) | 2.6222222 | 0.1916383 | 1451 | 13.683181 | 0 |
| ARSBFGLNGS30157G/A | 0.2459028 | 0.2805849 | 0 | 0.876393 | NaN |
| ARSBFGLNGS30157A/A | 0.9666667 | 0.5775666 | 0 | 1.673689 | NaN |

Correlation:

| | | |
|---|---|---|
| | (Intr) | ARSBFGLNGS30157G |
| ARSBFGLNGS30157G/A | −0.683 | |
| ARSBFGLNGS30157A/A | −0.332 | 0.227 |

Standardized Within-Group Residuals:

| | | | | |
|---|---|---|---|---|
| Min | Q1 | Med | Q3 | Max |
| −1.80186743 | −0.73049290 | −0.07955863 | 0.63646906 | 4.86754182 |

Number of Observations: 1454
Number of Groups: 3

| | | | | |
|---|---|---|---|---|
| | numDF | denDF | F-value | p-value |
| (Intercept) | 1 | 1451 | 423.4041 | <.0001 |
| ARSBFGLNGS30157 | 2 | 0 | 1.5309 | NaN |

ARSBFGLNGS30157 × HAPMAP30097BTC007678
Linear mixed-effects model fit by REML
Data: vm

| | | |
|---|---|---|
| AIC | BIC | logLik |
| 5388.715 | 5415.115 | −2689.357 |

Random effects:
Formula: ~1|HAPMAP30097BTC007678

| | | |
|---|---|---|
| | (Intercept) | Residual |
| StdDev: | 0.0001396805 | 1.536253 |

Fixed effects: list(fixed)

| | | | | | |
|---|---|---|---|---|---|
| | Value | Std. Error | DF | t-value | p-value |
| (Intercept) | 2.6222223 | 0.0458024 | 1449 | 57.25079 | 0.0000 |
| ARSBFGLNGS30157G/A | 0.2459028 | 0.0973297 | 1449 | 2.52649 | 0.0116 |
| ARSBFGLNGS30157A/A | 0.9666667 | 0.5141287 | 1449 | 1.88020 | 0.0603 |

Correlation:

| | | |
|---|---|---|
| | (Intr) | ARSBFGLNGS30157G |
| ARSBFGLNGS30157G/A | −0.471 | |
| ARSBFGLNGS30157A/A | −0.089 | 0.042 |

Standardized Within-Group Residuals:

| | | | | |
|---|---|---|---|---|
| Min | Q1 | Med | Q3 | Max |
| −1.80186740 | −0.73049303 | −0.07955876 | 0.63646894 | 4.86754185 |

Number of Observations: 1454
Number of Groups: 3

| | | | | |
|---|---|---|---|---|
| | numDF | denDF | F-value | p-value |
| (Intercept) | 1 | 1449 | 4432.591 | <.0001 |
| ARSBFGLNGS30157 | 2 | 1449 | 4.768 | 0.0086 |

HAPMAP30097BTC007678 × ARSBFGLNGS102860
Linear mixed-effects model fit by REML
Data: vm

| | | |
|---|---|---|
| AIC | BIC | logLik |
| 5394.222 | 5420.622 | −2692.111 |

Random effects:
Formula: ~1|ARSBFGLNGS102860

| | | |
|---|---|---|
| | (Intercept) | Residual |
| StdDev: | 0.1681513 | 1.537635 |

Fixed effects: list(fixed)

| | | | | | |
|---|---|---|---|---|---|
| | Value | Std. Error | DF | t-value | p-value |
| (Intercept) | 2.6151655 | 0.1150824 | 1449 | 22.724293 | 0.0000 |
| HAPMAP30097BTC007678C/T | 0.0510603 | 0.1108123 | 1449 | 0.460781 | 0.6450 |
| HAPMAP30097BTC007678T/T | 0.7890341 | 0.4461378 | 1449 | 1.768588 | 0.0772 |

Correlation:

| | | |
|---|---|---|
| | (Intr) | HAPMAP30097BTC007678C |
| HAPMAP30097BTC007678C/T | −0.160 | |
| HAPMAP30097BTC007678T/T | −0.042 | 0.039 |

Standardized Within-Group Residuals:

| | | | | |
|---|---|---|---|---|
| Min | Q1 | Med | Q3 | Max |
| −1.71632537 | −0.74399344 | −0.09364397 | 0.62413466 | 4.84901203 |

Number of Observations: 1454
Number of Groups: 3

| | | | | |
|---|---|---|---|---|
| | numDF | denDF | F-value | p-value |
| (Intercept) | 1 | 1449 | 537.0079 | <.0001 |
| HAPMAP30097BTC007678 | 2 | 1449 | 1.6405 | 0.1942 |

HAPMAP30097BTC007678 × BFGLNGS119018
Linear mixed-effects model fit by REML
Data: vm

| | | |
|---|---|---|
| AIC | BIC | logLik |
| 5394.381 | 5420.781 | −2692.191 |

Random effects:
Formula: ~1|BFGLNGS119018

| | | |
|---|---|---|
| | (Intercept) | Residual |
| StdDev: | 0.08085095 | 1.538697 |

Fixed effects: list(fixed)

| | | | | | |
|---|---|---|---|---|---|
| | Value | Std. Error | DF | t-value | p-value |
| (Intercept) | 2.6420821 | 0.0726076 | 1449 | 36.38852 | 0.0000 |
| HAPMAP30097BTC007678C/T | 0.0493730 | 0.1108659 | 1449 | 0.44534 | 0.6561 |
| HAPMAP30097BTC007678T/T | 0.7565027 | 0.4465641 | 1449 | 1.69405 | 0.0905 |

Correlation:

| | | |
|---|---|---|
| | (Intr) | HAPMAP30097BTC007678C |
| HAPMAP30097BTC007678C/T | −0.242 | |
| HAPMAP30097BTC007678T/T | −0.052 | 0.040 |

Standardized Within-Group Residuals:

| | | | | |
|---|---|---|---|---|
| Min | Q1 | Med | Q3 | Max |
| −1.7173434 | −0.7753953 | −0.1254948 | 0.6512757 | 4.8137487 |

Number of Observations: 1454
Number of Groups: 3

| | | | | |
|---|---|---|---|---|
| | numDF | denDF | F-value | p-value |
| (Intercept) | 1 | 1449 | 1422.7826 | <.0001 |
| HAPMAP30097BTC007678 | 2 | 1449 | 1.5066 | 0.222 |

HAPMAP30097BTC007678 × ARSBFGLBAC20850
Linear mixed-effects model fit by REML
Data: vm

| | | |
|---|---|---|
| AIC | BIC | logLik |
| 5395.103 | 5421.503 | −2692.551 |

Random effects:
Formula: ~1|ARSBFGLBAC20850

| | | |
|---|---|---|
| | (Intercept) | Residual |
| StdDev: | 0.0003628686 | 1.539624 |

Fixed effects: list(fixed)

| | | | | | |
|---|---|---|---|---|---|
| | Value | Std. Error | DF | t-value | p-value |
| (Intercept) | 2.6680126 | 0.0442077 | 1449 | 60.35175 | 0.0000 |
| HAPMAP30097BTC007678C/T | 0.0503284 | 0.1109307 | 1449 | 0.45369 | 0.6501 |
| HAPMAP30097BTC007678T/T | 0.7736541 | 0.4466443 | 1449 | 1.73215 | 0.0835 |

Correlation:

| | | |
|---|---|---|
| | (Intr) | HAPMAP30097BTC007678C |
| HAPMAP30097BTC007678C/T | −0.399 | |
| HAPMAP30097BTC007678T/T | −0.099 | 0.039 |

Standardized Within-Group Residuals:

| | | | | |
|---|---|---|---|---|
| Min | Q1 | Med | Q3 | Max |
| −1.7006372 | −0.7586354 | −0.1091262 | 0.6053340 | 4.8271443 |

Number of Observations: 1454
Number of Groups: 3

| | | | | |
|---|---|---|---|---|
| | numDF | denDF | F-value | p-value |
| (Intercept) | 1 | 1449 | 4412.979 | <.0001 |
| HAPMAP30097BTC007678 | 2 | 1449 | 1.575 | 0.2075 |

HAPMAP30097BTC007678 × ARSBFGLNGS100843
Linear mixed-effects model fit by REML
Data: vm

| | | |
|---|---|---|
| AIC | BIC | logLik |
| 5394.15 | 5420.55 | −2692.075 |

Random effects:
Formula: ~1|ARSBFGLNGS100843

| | | |
|---|---|---|
| | (Intercept) | Residual |
| StdDev: | 0.1174539 | 1.538469 |

Fixed effects: list(fixed)

| | | | | | |
|---|---|---|---|---|---|
| | Value | Std. Error | DF | t-value | p-value |
| (Intercept) | 2.7322505 | 0.0979922 | 1449 | 27.882340 | 0.0000 |
| HAPMAP30097BTC007678C/T | 0.0467127 | 0.1108818 | 1449 | 0.421284 | 0.6736 |
| HAPMAP30097BTC007678T/T | 0.7495515 | 0.4466490 | 1449 | 1.678167 | 0.0935 |

Correlation:

| | | |
|---|---|---|
| | (Intr) | HAPMAP30097BTC007678C |
| HAPMAP30097BTC007678C/T | −0.185 | |
| HAPMAP30097BTC007678T/T | −0.066 | 0.040 |

Standardized Within-Group Residuals:

| | | | | |
|---|---|---|---|---|
| Min | Q1 | Med | Q3 | Max |
| −1.74455288 | −0.74663743 | −0.09664057 | 0.61835597 | 4.84333555 |

Number of Observations: 1454
Number of Groups: 3

| | | | | |
|---|---|---|---|---|
| | numDF | denDF | F-value | p-value |
| (Intercept) | 1 | 1449 | 818.0351 | <.0001 |
| HAPMAP30097BTC007678 | 2 | 1449 | 1.4709 | 0.2301 |

HAPMAP30097BTC007678 × ARSBFGLNGS97162
Linear mixed-effects model fit by REML
Data: vm

| | | |
|---|---|---|
| AIC | BIC | logLik |
| 5390.649 | 5417.05 | −2690.325 |

Random effects:
Formula: ~1|ARSBFGLNGS97162

| | | |
|---|---|---|
| | (Intercept) | Residual |
| StdDev: | 0.1637822 | 1.536118 |

Fixed effects: list(fixed)

| | | | | | |
|---|---|---|---|---|---|
| | Value | Std. Error | DF | t-value | p-value |
| (Intercept) | 2.7390141 | 0.1225977 | 1449 | 22.341474 | 0.0000 |
| HAPMAP30097BTC007678C/T | 0.0529657 | 0.1106873 | 1449 | 0.478517 | 0.6324 |
| HAPMAP30097BTC007678T/T | 0.7661151 | 0.4458771 | 1449 | 1.718221 | 0.0860 |

Correlation:

| | | |
|---|---|---|
| | (Intr) | HAPMAP30097BTC007678C |
| HAPMAP30097BTC007678C/T | −0.146 | |
| HAPMAP30097BTC007678T/T | −0.053 | 0.040 |

Standardized Within-Group Residuals:

| | | | | |
|---|---|---|---|---|
| Min | Q1 | Med | Q3 | Max |
| −1.7704426 | −0.7288562 | −0.1042626 | 0.6382259 | 4.8777529 |

Number of Observations: 1454
Number of Groups: 3

| | | | | |
|---|---|---|---|---|
| | numDF | denDF | F-value | p-value |
| (Intercept) | 1 | 1449 | 517.9953 | <.0001 |
| HAPMAP30097BTC007678 | 2 | 1449 | 1.5605 | 0.2104 |

HAPMAP30097BTC007678 × Hapmap42294BTA69421
Linear mixed-effects model fit by REML
Data: vm

| | | |
|---|---|---|
| AIC | BIC | logLik |
| 5394.432 | 5420.832 | −2692.216 |

Random effects:
Formula: ~1|Hapmap42294BTA69421

| | | |
|---|---|---|
| | (Intercept) | Residual |
| StdDev: | 0.07699106 | 1.538480 |

Fixed effects: list(fixed)

| | | | | | |
|---|---|---|---|---|---|
| | Value | Std. Error | DF | t-value | p-value |
| (Intercept) | 2.6630614 | 0.0638045 | 1449 | 41.73786 | 0.0000 |
| HAPMAP30097BTC007678C/T | 0.0551102 | 0.1109015 | 1449 | 0.49693 | 0.6193 |
| HAPMAP30097BTC007678T/T | 0.7522181 | 0.4465602 | 1449 | 1.68447 | 0.0923 |

Correlation:

| | | |
|---|---|---|
| | (Intr) | HAPMAP30097BTC007678C |
| HAPMAP30097BTC007678C/T | −0.279 | |
| HAPMAP30097BTC007678T/T | −0.068 | 0.039 |

Standardized Within-Group Residuals:

| | | | | |
|---|---|---|---|---|
| Min | Q1 | Med | Q3 | Max |
| −1.74244914 | −0.74327146 | −0.09327913 | 0.62171242 | 4.79329529 |

Number of Observations: 1454
Number of Groups: 3

| | | | | |
|---|---|---|---|---|
| | numDF | denDF | F-value | p-value |
| (Intercept) | 1 | 1449 | 1917.4009 | <.0001 |
| HAPMAP30097BTC007678 | 2 | 1449 | 1.5122 | 0.2208 |

HAPMAP30097BTC007678 × ARSBFGLBAC2384
Linear mixed-effects model fit by REML
Data: vm

| | | |
|---|---|---|
| AIC | BIC | logLik |
| 5393.419 | 5419.819 | −2691.710 |

Random effects:
Formula: ~1|ARSBFGLBAC2384

| | | |
|---|---|---|
| | (Intercept) | Residual |
| StdDev: | 0.08924033 | 1.537870 |

Fixed effects: list(fixed)

| | | | | | |
|---|---|---|---|---|---|
| | Value | Std. Error | DF | t-value | p-value |
| (Intercept) | 2.6674431 | 0.0711772 | 1449 | 37.47610 | 0.0000 |
| HAPMAP30097BTC007678C/T | 0.0502424 | 0.1108068 | 1449 | 0.45342 | 0.6503 |
| HAPMAP30097BTC007678T/T | 0.7770507 | 0.4462109 | 1449 | 1.74144 | 0.0818 |

Correlation:

| | | |
|---|---|---|
| | (Intr) | HAPMAP30097BTC007678C |
| HAPMAP30097BTC007678C/T | −0.245 | |
| HAPMAP30097BTC007678T/T | −0.056 | 0.040 |

Standardized Within-Group Residuals:

| | | | | |
|---|---|---|---|---|
| Min | Q1 | Med | Q3 | Max |
| −1.7484251 | −0.7539616 | −0.1227997 | 0.6251456 | 4.7867467 |

Number of Observations: 1454
Number of Groups: 3

| | | | | |
|---|---|---|---|---|
| | numDF | denDF | F-value | p-value |
| (Intercept) | 1 | 1449 | 1512.8123 | <.0001 |
| HAPMAP30097BTC007678 | 2 | 1449 | 1.5904 | 0.2042 |

HAPMAP30097BTC007678 × BTB01553536
Linear mixed-effects model fit by REML
Data: vm

| | | |
|---|---|---|
| AIC | BIC | logLik |
| 5393.322 | 5419.722 | −2691.661 |

Random effects:
Formula: ~1|BTB01553536

| | | |
|---|---|---|
| | (Intercept) | Residual |
| StdDev: | 0.0906647 | 1.537678 |

Fixed effects: list(fixed)

| | | | | | |
|---|---|---|---|---|---|
| | Value | Std. Error | DF | t-value | p-value |
| (Intercept) | 2.6708128 | 0.0691938 | 1449 | 38.59901 | 0.0000 |
| HAPMAP30097BTC007678C/T | 0.0423921 | 0.1109487 | 1449 | 0.38209 | 0.7025 |
| HAPMAP30097BTC007678T/T | 0.7720480 | 0.4460887 | 1449 | 1.73071 | 0.0837 |

Correlation:

| | | |
|---|---|---|
| | (Intr) | HAPMAP30097BTC007678C |
| HAPMAP30097BTC007678C/T | −0.249 | |
| HAPMAP30097BTC007678T/T | −0.062 | 0.040 |

Standardized Within-Group Residuals:

| | | | | |
|---|---|---|---|---|
| Min | Q1 | Med | Q3 | Max |
| −1.7437094 | −0.7406439 | −0.1141912 | 0.6250514 | 4.7871704 |

Number of Observations: 1454
Number of Groups: 3

| | | | | |
|---|---|---|---|---|
| | numDF | denDF | F-value | p-value |
| (Intercept) | 1 | 1449 | 1608.5997 | <.0001 |
| HAPMAP30097BTC007678 | 2 | 1449 | 1.5469 | 0.2133 |

HAPMAP30097BTC007678 × HAPMAP53129RS29022984
Linear mixed-effects model fit by REML
Data: vm

| | | |
|---|---|---|
| AIC | BIC | logLik |
| 5389.142 | 5415.542 | −2689.571 |

Random effects:
Formula: ~1|HAPMAP53129RS29022984

| | | |
|---|---|---|
| | (Intercept) | Residual |
| StdDev: | 0.1677840 | 1.535203 |

Fixed effects: list(fixed)

| | | | | | |
|---|---|---|---|---|---|
| | Value | Std. Error | DF | t-value | p-value |
| (Intercept) | 2.595670 | 0.1195186 | 1449 | 21.717707 | 0.0000 |
| HAPMAP30097BTC007678C/T | 0.046930 | 0.1106779 | 1449 | 0.424023 | 0.6716 |
| HAPMAP30097BTC007678T/T | 0.713517 | 0.4458569 | 1449 | 1.600327 | 0.1097 |

Correlation:

| | | |
|---|---|---|
| | (Intr) | HAPMAP30097BTC007678C |
| HAPMAP30097BTC007678C/T | −0.131 | |
| HAPMAP30097BTC007678T/T | −0.024 | 0.040 |

Standardized Within-Group Residuals:

| | | | | |
|---|---|---|---|---|
| Min | Q1 | Med | Q3 | Max |
| −1.7424929 | −0.7348539 | −0.1140434 | 0.6330437 | 4.8018746 |

Number of Observations: 1454
Number of Groups: 3

| | | | | |
|---|---|---|---|---|
| | numDF | denDF | F-value | p-value |
| (Intercept) | 1 | 1449 | 483.8869 | <.0001 |
| HAPMAP30097BTC007678 | 2 | 1449 | 1.3454 | 0.2608 |

HAPMAP30097BTC007678 × ARSBFGLNGS68110
Linear mixed-effects model fit by REML
Data: vm

| | | |
|---|---|---|
| AIC | BIC | logLik |
| 5358.232 | 5384.633 | −2674.116 |

Random effects:
Formula: ~1|ARSBFGLNGS68110

| | | |
|---|---|---|
| | (Intercept) | Residual |
| StdDev: | 0.3144339 | 1.517087 |

Fixed effects: list(fixed)

| | | | | | |
|---|---|---|---|---|---|
| | Value | Std. Error | DF | t-value | p-value |
| (Intercept) | 2.6360428 | 0.1874689 | 1449 | 14.061229 | 0.0000 |
| HAPMAP30097BTC007678C/T | 0.0221465 | 0.1094197 | 1449 | 0.202399 | 0.8396 |
| HAPMAP30097BTC007678T/T | 0.7588728 | 0.4401125 | 1449 | 1.724270 | 0.0849 |

Correlation:

| | | |
|---|---|---|
| | (Intr) | HAPMAP30097BTC007678C |
| HAPMAP30097BTC007678C/T | −0.089 | |
| HAPMAP30097BTC007678T/T | −0.023 | 0.040 |

Standardized Within-Group Residuals:

| | | | | |
|---|---|---|---|---|
| Min | Q1 | Med | Q3 | Max |
| −1.9187945 | −0.7177124 | −0.1122266 | 0.6006033 | 4.6873817 |

Number of Observations: 1454
Number of Groups: 3

| | | | | |
|---|---|---|---|---|
| | numDF | denDF | F-value | p-value |
| (Intercept) | 1 | 1449 | 200.85317 | <.0001 |
| HAPMAP30097BTC007678 | 2 | 1449 | 1.49557 | 0.2245 |

HAPMAP30097BTC007678 × HAPMAP49592BTA38891
Linear mixed-effects model fit by REML
Data: vm

| | | |
|---|---|---|
| AIC | BIC | logLik |
| 5395.039 | 5421.439 | −2692.519 |

Random effects:
Formula: ~1|HAPMAP49592BTA38891

| | | |
|---|---|---|
| | (Intercept) | Residual |
| StdDev: | 0.05669389 | 1.539240 |

Fixed effects: list(fixed)

| | | | | | |
|---|---|---|---|---|---|
| | Value | Std. Error | DF | t-value | p-value |
| (Intercept) | 2.6657425 | 0.0599806 | 1449 | 44.44341 | 0.0000 |
| HAPMAP30097BTC007678C/T | 0.0483156 | 0.1109398 | 1449 | 0.43551 | 0.6633 |
| HAPMAP30097BTC007678T/T | 0.7753004 | 0.4465479 | 1449 | 1.73621 | 0.0827 |

Correlation:

| | | |
|---|---|---|
| | (Intr) | HAPMAP30097BTC007678C |
| HAPMAP30097BTC007678C/T | −0.298 | |
| HAPMAP30097BTC007678T/T | −0.076 | 0.039 |

Standardized Within-Group Residuals:

| | | | | |
|---|---|---|---|---|
| Min | Q1 | Med | Q3 | Max |
| −1.7190499 | −0.7521092 | −0.1024380 | 0.6122003 | 4.8350628 |

Number of Observations: 1454
Number of Groups: 3

| | | | | |
|---|---|---|---|---|
| | numDF | denDF | F-value | p-value |
| (Intercept) | 1 | 1449 | 2201.8391 | <.0001 |
| HAPMAP30097BTC007678 | 2 | 1449 | 1.5747 | 0.2074 |

HAPMAP30097BTC007678 × ARSBFGLNGS30157
Linear mixed-effects model fit by REML
Data: vm

| | | |
|---|---|---|
| AIC | BIC | logLik |
| 5391.111 | 5417.511 | −2690.555 |

Random effects:
Formula: ~1|ARSBFGLNGS30157

| | | |
|---|---|---|
| | (Intercept) | Residual |
| StdDev: | 0.1934441 | 1.536247 |

Fixed effects: list(fixed)

| | | | | | |
|---|---|---|---|---|---|
| | Value | Std. Error | DF | t-value | p-value |
| (Intercept) | 2.7788558 | 0.1411107 | 1449 | 19.692739 | 0.0000 |
| HAPMAP30097BTC007678C/T | 0.0513359 | 0.1106983 | 1449 | 0.463746 | 0.6429 |
| HAPMAR30097BTC007678T/T | 0.7502175 | 0.4457845 | 1449 | 1.682915 | 0.0926 |

Correlation:

| | | |
|---|---|---|
| | (Intr) | HAPMAP30097BTC007678C |
| HAPMAP30097BTC007678C/T | −0.118 | |
| HAPMAP30097BTC007678T/T | −0.033 | 0.039 |

Standardized Within-Group Residuals:

| | | | | |
|---|---|---|---|---|
| Min | Q1 | Med | Q3 | Max |
| −1.78275989 | −0.74126063 | −0.09032359 | 0.62570716 | 4.87044496 |

Number of Observations: 1454
Number of Groups: 3

| | | | | |
|---|---|---|---|---|
| | numDF | denDF | F-value | p-value |
| (Intercept) | 1 | 1449 | 397.6386 | <.0001 |
| HAPMAP30097BTC007678 | 2 | 1449 | 1.4951 | 0.2246 |

HAPMAP30097BTC007678 × HAPMAP30097BTC007678
Linear mixed-effects model fit by REML
Data: vm

| | | |
|---|---|---|
| AIC | BIC | logLik |
| 5395.103 | 5421.503 | −2692.551 |

Random effects:
Formula: ~1|HAPMAP30097BTC007678

| | | |
|---|---|---|
| | (Intercept) | Residual |
| StdDev: | 0.1864927 | 1.539624 |

Fixed effects: list(fixed)

| | | | | | |
|---|---|---|---|---|---|
| | Value | Std. Error | DF | t-value | p-value |
| (Intercept) | 2.6680132 | 0.1916604 | 1451 | 13.920522 | 0 |
| HAPMAP30097BTC007678C/T | 0.0503274 | 0.2861198 | 0 | 0.175896 | NaN |
| HAPMAP30097BTC007678T/T | 0.7736535 | 0.5187004 | 0 | 1.491523 | NaN |

Correlation:

| | | |
|---|---|---|
| | (Intr) | HAPMAP30097BTC007678C |
| HAPMAP30097BTC007678C/T | −0.670 | |
| HAPMAP30097BTC007678T/T | −0.370 | 0.248 |

Standardized Within-Group Residuals:

| | | | | |
|---|---|---|---|---|
| Min | Q1 | Med | Q3 | Max |
| −1.7006365 | −0.7586354 | −0.1091261 | 0.6053341 | 4.8271443 |

Number of Observations: 1454
Number of Groups: 3

| | | | | |
|---|---|---|---|---|
| | numDF | denDF | F-value | p-value |
| (Intercept) | 1 | 1451 | 406.2358 | <.0001 |
| HAPMAP30097BTC007678 | 2 | 0 | 1.1322 | NaN |

1. A method of predicting the phenotype of an animal comprising:
selecting a phenotypic trait in a population of animals;
determining single nucleotide polymorphisms in the genotype of the population of animals,
correlating the single nucleotide polymorphisms with the phenotypic trait, and
predicting the phenotype of the animal based on the results of the correlation.

2. A method of predicting the tolerance of a cow to stress comprising:
determining cortisol levels in a population of cattle;
determining single nucleotide polymorphisms in the cattle genome;
correlating the single nucleotide polymorphisms with the cortisol levels in the cattle; and
predicting the cortisol level in a cow based on the results of the correlation.

3. A method for predicting a phenotypic trait in a cow comprising: determining the nucleotide present at a locus selected from the group consisting of ARS-BFGL-NGS-102860 mapped at position 36,875,752 (Btau4.0) of bovine chromosome 16 (BTA16); ARS-BFGL-NGS-119018 mapped at position 104,533,532 (Btau4.0) of bovine chromosome 11 (BTA11), ARS-BFGL-NGS-20850 at position 7,928,145 (Btau4.0) of bovine chromosome 14 (BTA14), ARS-BFGL-NGS-100843 mapped at position 45,768,092 (Btau4.0) of bovine chromosome 16 (BTA16), ARS-BFGL-NGS-97162 mapped at position 51,027,089 (Btau4.0) of bovine chromosome 16 (BTA16), Hapmap42294-BTA-69421 at position 7,311,099 (Btau4.0) of bovine chromosome 3 (BTA3), ARS-BFGL-BAC-2384 at position 31,838,306 (Btau4.0) of bovine chromosome 19 (BTA19), BTB-01553536 at position 103,411,819 (Btau4.0) of bovine chromosome 7 (BTA7) Hapmap53129-rs29022984 at position 97,865,487 (Btau4.0) of bovine chromosome 11 (BTA11); ARS-BFGL-NGS-68110 mapped at position 106,356,144 (Btau4.0) of bovine chromosome 11 (BTA11); Hapmap49592-BTA-38891 at position 36,808,659 (Btau4.0) of bovine chromosome 16 (BTA16); ARS-BFGL-NGS-30157 mapped at position 108,365,498 (Btau4.0) of bovine chromosome 11 (BTA11); Hapmap30097-BTC-007678 mapped at position 7,969,430 (Btau4.0) of bovine chromosome 14 (BTA14); ARS-BFGL-NGS-82206 mapped at position 130,073,477 (Btau4.0) of bovine chromosome 1, ARS-BFGL-NGS-114897 mapped at position 69,718,192 (Btau4.0) of bovine chromosome 11 (BTA11), ARS-BFGL-NGS-32646 mapped at position 103,515,296 (Btau4.0) of bovine chromosome 11 (BTA11); ARS-BFGL-NGS-12135 mapped at position 106,208,942 (Btau4.0) of bovine chromosome 11 (BTA11), BTA-98582-no-rs mapped at position 72,891,230 (Btau4.0) of bovine chromosome 15 (BTA15), Hapmap50501-BTA-91866 mapped at position 16,697,957 (Btau4.0) of bovine chromosome 16 (BTA16), ARS-BFGL-NGS-55834 mapped at position 18,500,742 (Btau4.0) of bovine chromosome 16 (BTA16), ARS-BFGL-NGS-43639 at position 45,798,238 (Btau4.0) of bovine chromosome 16 (BTA16), ARS-BFGL-NGS-114602 mapped at position 2,011,968 (Btau4.0) of bovine chromosome 20 (BTA20), ARS-BFGL-NGS-10830 mapped at position 14,303,665 (Btau4.0) of bovine chromosome 21 (BTA21), ARS-BFGL-BAC-35732 mapped at position 37,243,031 (Btau4.0) of bovine chromosome 22 (BTA22), BTB-00000725 mapped at position 19,405,585 (Btau4.0) of bovine chromosome 27 (BTA27), Hapmap32414-BTA-65998 mapped at position 38,481,013 (Btau4.0) of bovine chromosome 28 (BTA28), Hapmap26724-BTA-152272 mapped at position 126,295,740 (Btau4.0) of bovine chromosome 1 (BTA1), ARS-BFGL-NGS-27655 mapped at position 3,683,167 (Btau4.0) of bovine chromosome 3 (BTA3), ARS-BFGL-NGS-112731 mapped at position 4,206,765 (Btau4.0) of bovine chromosome 2 (BTA2), Hapmap42580-BTA-54259 mapped at position 38555445 (Btau4.0) of bovine chromosome 22 (BTA22), BTB-01548453 mapped at position 103,511,536 (Btau4.0) of bovine chromosome 7 (BTA7), INRA-453 mapped at position 20,719,615 (Btau4.0) of bovine chromosome 3 (BTA3), BTB-00186413 mapped at position 58,422,144 (Btau4.0) of bovine chromosome 4 (BTA4)(G), UA-IFASA-7842 at position 7,857,978 (Btau4.0) of bovine chromosome 14 (BTA14), BTB-01944037 at position 112,370,482 (Btau4.0) of bovine chromosome 8 (BTA8), BTB-00086583 at position 26,641,920 (Btau4.0) of bovine chromosome 2 (BTA2), ARS-BFGL-NGS-111311 at position 51,300,416 (Btau4.0) of bovine chromosome 23 (BTA23), BTB-01570493 at position 25,395,611 (Btau4.0) of bovine chromosome 8 (BTA8), ARS-BFGL-NGS-104914 at position 125,588,038 (Btau4.0) of bovine chromosome 5 (BTA5). BTA-114011-no-rs at position 125,911,737 (Btau4.0) of bovine chromosome 1 (BTA1), ARS-BFGL-NGS-23375 at position 40,238,627 (Btau4.0) of bovine chromosome 24 (BTA24), ARS-BFGL-NGS-78666 at position 136,573,912 (Btau4.0) of bovine chromosome 1 (BTA1), BTB-01087838 at position 89,620,818 (Btau4.0) of bovine chromosome 10 (BTA10), Hapmap31564-BTC-007633 at position 7,998,737 (Btau4.0) of bovine chromosome 14 (BTA14), Hapmap50402-BTA-58146 at position 42,593,193 (Btau4.0) of bovine chromosome 24 (BTA24), and ARS-BFGL-BAC-46971 at position 35,184,932 (Btau4.0) of bovine chromosome 25 (BTA25), either alone or in combination with other loci, and predicting the phenotypic trait in the cow comprising based on the nucleotide present at the locus.

4. The method of claim 3, wherein the step of determining the nucleotide present in each allele of the locus is performed by genomic DNA sequencing of a region which includes the locus.

5. The method of claim 3, wherein the step of determining the nucleotide present in each allele of the locus comprises:
(a) amplifying a region of genomic DNA that includes the locus to generate an amplicon, and
(b) treating the amplicon with a restriction enzyme in its corresponding buffer to determine the identity of the nucleotides present in the selected locus.

6. The method of claim 3, wherein the step of determining the nucleotide present in allele at the locus comprises:
(a) amplifying a region of genomic DNA that includes the given position to generate an amplicon, and
(b) hybridizing an amplified probe specific to the selected locus, wherein hybridization determines the identity of the nucleotides present.
